(19) United States
(12) Patent Application Publication
(10) Pub. No.: US 2023/0330064 A1
(43) Pub. Date: Oct. 19, 2023

(54) TYROSINE, TRYPTOPHAN AND PHENYLALANINE AS mTOR AGONISTS MEDIATING PROTEASOME DYNAMICS, COMPOSITIONS, METHODS AND USES THEREOF IN THERAPY, AND PROGNOSTIC METHODS FOR DRUG-RESISTANCE

(71) Applicants: Technion Research & Development Foundation Limited, Haifa (IL); RAMBAM MED-TECH LTD., Haifa (IL)

(72) Inventors: Ido LIVNEH, Haifa (IL); Victoria COHEN-KAPLAN, Haifa (IL); Aaron CIECHANOVER, Caesarea (IL); Gilad GOLDHIRSH, Kfar Saba (IL); Yaniv ZOHAR, Kiryat Tivon (IL); Noa LAVI, Karkur (IL); Bertrand Francois FABRE, Ramonville Saint-Agne (FR)

(21) Appl. No.: 18/004,821

(22) PCT Filed: Jul. 8, 2021
(86) PCT No.: PCT/IL2021/050842

§ 371 Date: Jan. 9, 2023

Related U.S. Application Data
(60) Provisional application No. 62/705,668, filed on Jul. 9, 2020.
(60) Provisional application No. 63/147,888, filed on Feb. 10, 2021.

(51) Int. Cl. A61K 31/405; A61P 35/00; A61K 31/198; G01N 33/50

(52) U.S. Cl. CPC A61K 31/405; A61P 35/00; A61K 31/198; G01N 33/5035; G01N 33/5082

(57) ABSTRACT

The present disclosure provides mTOR agonists that selectively modulate proteasome dynamics, compositions, methods and uses thereof for modulation of stress-induced proteasome dynamics and related pathological conditions. The present disclosure specifically provides therapeutic methods for treating disorders associated with cytosolic accumulation of the proteasome. The invention further provides prognostic methods for detection and monitoring drug resistant cancers, as well as methods for screening for modulators of proteasome dynamics.

The Sequence Listing in ASCII text file format of 3,189 bytes in size, created on Jun. 28, 2023, with the file name “2023-06-29SequenceListing_LIVNEH2A,” filed in the U.S. Patent and Trademark Office on even date herewith, is hereby incorporated herein by reference.

### FIELD OF THE INVENTION

The invention relates to the field of therapeutic and prognostic compounds, compositions and methods, and application thereof for conditions associated with proteasome dynamics. More specifically, the invention provides mTOR agonists that selectively modulate proteasome dynamics, compositions, methods and uses thereof for modulation of stress-induced proteasome dynamics and related pathological conditions. The invention further provides prognostic methods for detection and monitoring drug resistant cancers.

### BACKGROUND ART

References considered to be relevant as background to the presently disclosed subject matter are listed below:
[1] Cohen-Kaplan, V., Livneh, I., Avni, N., Fabre, B., Ziv, T., Kwon, Y. T., and Ciechanover, A. (2016a). p62- and ubiquitin-dependent stress-induced autophagy of the mammalian 26S proteasome. Proc. Natl. Acad. Sci. 113, E7490-99.
[2] Marshall, R. S., Li, F., Gemperline, D. C., Book, A. J., and Vierstra, R. D. (2015). Autophagic Degradation of the 26S Proteasome Is Mediated by the Dual ATG8/Ubiquitin Receptor RPN10 in Arabidopsis. Mol. Cell 58, 1053-1066.
[3] Waite, K. A., De La Mota-Peynado, A., Vontz, G., Roelofs, J., De-La Mota-Peynado, A., Vontz, G., and Roelofs, J. (2015). Starvation Induces Proteasome Autophagy with Different Pathways for Core and Regulatory Particle. J. Biol. Chem. 291, 3239-3253.
[4] Yasuda, S., Tsuchiya, H., Kaiho, A., Guo, Q., Ikeuchi, K., Endo, A., Arai, N., Ohtake, F., Murata, S., Inada, T., et al. (2020). Stress- and ubiquitylation-dependent phase separation of the proteasome. Nature 578, 296-300.
[5] Burcoglu J. et al., (2015) Cells 4, 387-405.
[6] Saxton, R. A. et al., (2017) Cell 168, 960-976.
[7] Wullschleger, S. et al., (2006) Cell 124, 471-484.
[8] Takahara, T. et al., (2020) J. Biomed. Sci. 27, 1-16.
[9] Zhao, J. et al., (2015) Proc. Natl. Acad. Sci. 112, 15790-15797.
[10] Rousseau, A. et al., (2016) Nature 536, 184-189.
[11] Zhang Y. et al., (2014) Nature 513, 440-443.
[12] Deng, K. et al., (2012) Plos one 7(11) e49434.
[13] Christoph Giese, et al., (2008) ChemMedChem, 3, 1449-1456.
[14] WO15137383 A1.
[15] US2005119256 AA.
[16] WO2008081537A1
[17] Cohen-kaplan, V., Livneh, I., Avni, N., Cohen-Rosenzweig, C., and Ciechanover, A. (2016). The ubiquitin-proteasome system and autophagy: Coordinated and independent activities. Int. J. Biochem. Cell Biol. 79, 403-418.
[18] Dikic, I. (2017). Proteasomal and Autophagic Degradation Systems. Annu. Rev. Biochem. 86, 193-224.
[19] Manasanch, E. E., and Orlowski, R. Z. (2017). Proteasome inhibitors in cancer therapy. Nat. Rev. Clin. Oncol. 14, 417-433.
[20] Slater, A. F. G. (1993). Chloroquine: Mechanism of Drug Action and Resistance in Plasmodium Falcipar Um. Pharmac. Ther 57, 203-235.
[21] Russell, S. J., Steger, K. a., and Johnston, S. A. (1999). Subcellular localization, stoichiometry, and protein levels of 26 S proteasome subunits in yeast. J. Biol. Chem. 274, 21943-21952.
[22] Heitman, J., Movva, N. R., and Hall, M. N. (1991). Targets for cell cycle arrest by the immunosuppressant rapamycin in yeast. Science (80-.). 253, 905-909.
[23] Sabatini, D. M., Erdjument-Bromage, H., Lui, M., Tempst, P., and Snyder, S. H. (1994). RAFT1: A mammalian protein that binds to FKBP12 in a rapamycin-dependent fashion and is homologous to yeast TORs. Cell 78, 35-43.
[24] Cohen-Kaplan, V., Livneh, I., Avni, N., Cohen-Rosenzweig, C., and Ciechanover, A. (2016b). The ubiquitin-proteasome system and autophagy: Coordinated and independent activities. Int. J. Biochem. Cell Biol. 79.
[25] Shabaneh, T. B., Downey, S. L., Goddard, A. L., Screen, M., Lucas, M. M., Eastman, A., and Kisselev, A. F. (2013). Molecular Basis of Differential Sensitivity of Myeloma Cells to Clinically Relevant Bolus Treatment with Bortezomib. PLoS One 8, e56132.

Acknowledgement of the above references herein is not to be inferred as meaning that these are in any way relevant to the patentability of the presently disclosed subject matter.

### BACKGROUND OF THE INVENTION

The proteasome, the catalytic arm of the ubiquitin-proteasome system (UPS), is responsible for the removal of ubiquitinated proteins. Studying the fate of the 26S proteasome under stress, it was previously shown that following a long (˜24 hours) amino acids starvation, it undergoes autophagy [1-3]. While several aspects of proteasome regulation (e.g. assembly, composition and posttranslational modifications) have been unraveled, the question of its compartmentalization and adaptive concentration in response to environmental cues is just starting to emerge. For example, a recent study showed that osmotic stress induces generation of membraneless nuclear foci that contain high concentration of the proteasome and serve as proteolytic centers [4]. In yeast, proteasomes were shown to accumulate in cytosolic granules under shortage of glucose, but not that of amino acids, and this mechanism was shown to act as a protective measure against degradation of the proteasome, rather than as a proteolytic means to mitigate the stress itself [5]. One of the key regulators of amino acid shortage is the target of rapamycin (TOR), and its mammalian homolog known as the mechanistic TOR (mTOR). In the lack of nutrients, mTOR is inactive, resulting in upregulation of autophagy, which in turn supplies the cell with recycled building blocks [6, 7]. Characterization of the direct sensors through which the level of different amino acids is relayed to mTOR is still in its early stage, and only a handful of such proteins have been identified [8]. Similarly, it was not until recent years that a link between mTOR and the UPS was described: mTOR inhibition was shown to upregulate the UPS and proteasome activity, alongside with autophagy [9], and to stimulate proteasome assembly [10]. The relationship between the two pathways may depend on the pathophysiologic conditions, as a different contradicting study suggested that inhibition of mTOR leads to downregulation of the proteasome proteolytic activity [11].

The effect of aromatic amino acids on cancer cell growth has been previously described. Deng et al., [12], reported the detection of increased levels of tyrosine, phenylalanine and tryptophan in gastric juice samples of early phase of gastric carcinogenesis. Deng et al., further suggests the use of these aromatic amino acids as biomarkers for the early detection of gastric cancer. Similarly, Giese et al., [13], report that depletion of tryptophan (Trp, W), phenylalanine (Phe, F) or tyrosine (Tyr, Y), significantly reduces the growth of the breast cancer cell line MCF-7. In contrast, the presence of fluorinated aromatic amino acids, specifically, L-(4-F) Trp, completely inhibited the growth of these cells, in an irreversible manner. Giese et al., further indicate that the inhibitory activity of L-(4-F) Trp was only slightly reduced by the addition of unmodified L-Trp, suggesting that the growth inhibitory effect of L-(4-F) Trp cannot be easily remedied. This publication therefore suggests the use of fluorinate aromatic amino acids, specifically, the potent analogue L-(4-F) Trp, as a cytostatic and anti-tumor agent. According to this publication, the fluorinated derivatives are limited for local application only, as systemic administration of L-(4-F) Tip, may lead to sever side effects.

WO15137383 A1 [14], discloses the use of a glutamine metabolism inhibitor as a chemotherapy adjuvant. The glutamine metabolism inhibitor disclosed therein is an aromatic amino acid, specifically, L-phenylalanine, administered at a high concentration of 45 mM.

US2005119256 AA [15], discloses various derivatives of aromatic amino acids, and uses thereof as inhibitors of L-type amino acid transporter 1 (LAT-1), that is a cancer specific membrane protein required for intracellular uptake of essential amino acids. Particular effective derivatives disclosed by this publication, include 3, 5-Dichloro-O-[(2-phenyl)-benzoxazol-7-yl] methyl-L-tyrosine methyl ester hydrochloride, and 3-(2-naphthyloxy)-L-phenylalanine. Inhibition of LAT-1 by both derivatives resulted in reduction of intracellular ¹⁴C-Leucine, inhibition of the human bladder cancer T24 cell line proliferation and inhibition of tumor growth. Similarly, WO08081537 A1 [16], discloses various derivatives of aromatic amino acids and uses thereof as inhibitors of L-type amino acid transporter 1 (LAT-1).

Still further, besides of the removal of the proteasome by autophagy, the ‘canonical’ view is that the two proteolytic systems fulfill distinct physiological roles: whereas the UPS is responsible for specific and timed degradation of cellular proteins—e.g., transcription factors, cell cycle regulators, mutated and misfolded proteins—autophagy is responsible bulk removal of organelles and machineries largely under stress [17-18]. Given the wide involvement of these two systems in cellular processes, they also serve as drug development targets. For example, Chloroquine is used in malaria and autoimmune diseases via interfering with lysosomal activity, and proteasome inhibitors serve as first line treatment in Multiple Myeloma (MM) and amyloidosis. Interestingly, while both groups of drugs are widely used, their exact mechanisms of action are still elusive as are the mechanisms that underlie drug resistance [19-20].

Proteasome inhibitors constitute nowadays the first line treatment in multiple myeloma (which comprises 3% of all malignancies and 20% of hematological malignancies). A significant fraction of patients do not respond to the treatment, which costs precious time (and money), while exposing patients to adverse side effects and postponing initiation of other potential lines of treatment. To date, there are no reliable predictive tools as for the chances of a single patient to adequately respond to the drug. Additionally, clinical trials using novel drugs, may be ethically bound to treat also with proteasome inhibitors, as there is no way to predict which patients will not benefit from them. Medical indications for use of proteasome inhibitors are currently expanding, with recent addition of several oncologic and inflammatory diseases, while others under clinical trials. There is therefore need for powerful selective modulators of proteasome dynamics for use in therapy and diagnosis. These unmet needs are addressed by the present disclosure.

### SUMMARY OF THE INVENTION

A first aspect of the present disclosure relates to a mammalian target of rapamycin (mTOR) agonist comprising at least two aromatic amino acid residues or a combination of at least two aromatic amino acid residues or any mimetics thereof, any compound that modulates directly or indirectly at least one of the levels, stability and bioavailability of at least one of said aromatic amino acid residue, any combinations or mixtures thereof, or any vehicle, matrix, nano- or micro-particle thereof. In some specific embodiments, the mTOR agonist of the invention may comprise at least one of:

First (a), at least one tyrosine (Y) residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist may comprise in some embodiments (b), at least one tryptophan (W) residue, any mTOR agonistic tryptophan mimetic, any salt or ester thereof, any multimeric and/or polymeric form of the tryptophan residue and/or of the mTOR agonistic tryptophan mimetic, or any combination or mixture thereof. In yet some further embodiments, the mTOR agonist of the present disclosure may comprise (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof. In some specific embodiments, the mTOR agonist of the present disclosure may comprise at least one tyrosine (Y) residue, at least one tryptophan (W) residue, and at least one phenylalanine (F) residue, or any mTOR agonistic mimetic, salt or ester thereof, any multimeric and/or polymeric form thereof, and any combinations or mixtures thereof.

In a further aspect, the invention relates to a composition comprising as an active ingredient at least one mTOR agonist comprising at least two aromatic amino acid residues, any compound that modulates directly or indirectly at least one of the levels, stability and bioavailability of at least one of said aromatic amino acid residue, any combinations or mixtures thereof, any vehicle, matrix, nano- or micro-particle thereof, optionally, in at least one dosage unit form. In some embodiments, the composition may optionally further comprise at least one pharmaceutically acceptable carrier/s, excipient/s, auxiliaries, and/or diluent/s. In yet some further specific embodiments, the composition of the present disclosure comprises at least one tyrosine (Y) residue, at least one tryptophan (W) residue, and at least one phenylalanine (F) residue, or any mTOR agonistic mimetic, salt or ester thereof, any multimeric and/or polymeric form thereof, and any combinations or mixtures thereof, and any dosage unit form thereof. In some embodiments the mTOR agonist of the present disclosure is comprised in said composition in an amount effective for selective inhibition of proteasome translocation.

A further aspect of the invention relates to a kit comprising at least two of:

First (a), at least one tyrosine residue, any mTOR agonistic tyrosine mimetic, any salt or ester thereof, any multimeric and/or polymeric form of the tyrosine residue and/or of the mTOR agonistic tyrosine mimetic, any compound that modulates directly or indirectly at least one of the levels, stability and bioavailability of the tyrosine residue, and any combinations or mixtures thereof, optionally, in a first dosage form. In some embodiments, the kits of the invention may comprise additionally, or alternatively, (b), at least one tryptophan residue, any mTOR agonistic tryptophan mimetic, any salt or ester thereof, any multimeric and/or polymeric form of the tryptophan residue and/or of the mTOR agonistic tryptophan mimetic, any compound that modulates directly or indirectly at least one of the levels, stability and bioavailability of the tryptophan residue, or any combination or mixture thereof, optionally, in a second dosage form.

In yet some further embodiments, the kit of the invention may comprise additionally, or alternatively (c), at least one phenylalanine residue, any mTOR agonistic phenylalanine mimetic, any salt or ester thereof, any multimeric and/or polymeric form of the phenylalanine residue and/or of said mTOR agonistic phenylalanine mimetic, any compound that modulates directly or indirectly at least one of the levels, stability and bioavailability of the phenylalanine residue, and any combinations or mixtures thereof, optionally, in a third dosage form. In some embodiments, the kit of the present disclosure comprises all three aromatic amino acid residues, specifically, at least one tyrosine (Y) residue, at least one tryptophan (W) residue, and at least one phenylalanine (F) residue, or any mTOR agonistic mimetic, salt or ester thereof, any multimeric and/or polymeric form thereof, and any combinations or mixtures thereof, and any dosage unit form thereof.

Another aspect of the invention relates to a method for treating, preventing, inhibiting, reducing, eliminating, protecting or delaying the onset of at least one condition or at least one pathologic disorder associated with cytosolic proteasomal localization and/or activity in a subject. More specifically, the method comprises the step of administering to the subject an effective amount of at least one mTOR agonist comprising at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compound that modulates directly or indirectly at least one of the levels, stability and bioavailability of the at least one aromatic amino acid residue, any combinations or mixtures thereof, any vehicle, matrix, nano- or micro-particle thereof, any combinations or mixtures thereof, any vehicle, matrix, nano- or micro-particle thereof, any dosage form thereof, or any composition or kit comprising the at least one mTOR agonist.

A further aspect of the invention relates to an effective amount of at least one mTOR agonist for use in a method for treating, preventing, inhibiting, reducing, eliminating, protecting or delaying the onset of at least one condition or at least one pathologic disorder associated with cytosolic proteasomal localization and/or activity in a subject.

In a further aspect thereof, the present disclosure relates to a method for modulating a biological process associated directly or indirectly with proteasome dynamics in at least one cell and/or a subject. According to some embodiments, the methods comprise the step of contacting the at least one cell and/or administering to the subject a therapeutically effective amount of at least one mTOR agonist comprising at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compound that modulates directly or indirectly at least one of the levels, stability and bioavailability of the at least one aromatic amino acid residue, any combinations or mixtures thereof, any vehicle, matrix, nano- or micro-particle thereof, any combinations or mixtures thereof, any vehicle, matrix, nano- or micro-particle thereof, any dosage form thereof, or any composition or kit comprising the at least one mTOR agonist.

A further aspect of the invention relates to a prognostic method for predicting and assessing responsiveness of a subject suffering from a pathologic disorder to a treatment regimen comprising at least one ubiquitin proteasome system (UPS)-modulating agent, for example, at least one proteasome inhibitor, and optionally for monitoring disease progression. More specifically, in some embodiments the methods provided herein may comprise the following steps. In a first step (a), determining proteasome subcellular localization in at least one cell of at least one biological sample of the subject or in any fraction of the cell. The second step (b), involves classifying the subject as: (i), a responsive subject to the treatment regimen, if proteasome subcellular localization is predominantly nuclear in at least one cell of the at least one sample. Alternatively, the subject may be classified as (ii), a drug-resistant subject if proteasome subcellular localization is cytosolic. A further aspect of the invention relates to a method for determining a personalized treatment regimen for a subject suffering from a pathologic disorder. More specifically, the method of the invention may comprise the following steps: First in step (a), determining proteasome subcellular localization in at least one cell of at least one biological sample of the subject, or in any fraction of the cell. The next step (b), involves classifying said subject as: (i) a responsive subject to at least one treatment regimen comprising at least one UPS-modulating agent, for example, at least one proteasome inhibitor, if proteasome subcellular localization is predominantly nuclear, or (ii) a drug-resistant subject, to the treatment regimen, if proteasome subcellular localization is cytosolic. In some embodiments, subjects that display in at least one cell of at least one sample, both, nuclear and cytosolic proteasome localization, are classified as drug-resistant or as non-responders, if only 50% or less of the proteasome in at least one cell of said sample displays a nuclear localization. The next step (c), involves the selection of an appropriate treatment regimen. Specifically, in some embodiments, a subject classified as a responder is administered with an effective amount of at least one UPS-modulating agent, for example, at least one proteasome inhibitor, any combinations thereof or any compositions comprising the same.

In some other embodiments, subjects classified as drug-resistant or as non-responders will not be treated with the at least one proteasome inhibitor. In yet some further embodiments, for such non-responder subjects, a treatment regimen comprising at least one selective inhibitor of proteasome translocation, may be offered. In some embodiments, such selective inhibitor of proteasome translocation may comprise at least one mTOR agonist, as further discussed by the present disclosure.

A further aspect of the invention relates to a method for treating, preventing, inhibiting, reducing, eliminating, protecting or delaying the onset of at least one of, at least one proliferative disorder and at least one protein misfolding disorder in a subject in need thereof. More specifically, the therapeutic methods of the invention may comprise the following steps: First in step (a), determining proteasome subcellular localization in at least one cell of at least one biological sample of the subject, or in any fraction of the cell. In the next step (b), classifying the subject as: (i), a responsive subject to a treatment regimen comprising at least one UPS-modulating agent, for example, at least one proteasome inhibitor, if proteasome subcellular localization is predominantly nuclear, or (ii) a drug-resistant subject if proteasome subcellular localization is cytosolic. The next step (c), involves selecting a treatment regimen based on the responsiveness, thereby treating said subject. In some embodiments, this step further comprises applying the appropriate therapeutic regimen to the subject. In some specific embodiments, the appropriate treatment regimen may comprise at least one selective inhibitor of proteasome translocation, e.g., at least one mTOR agonist as disclosed herein.

In yet a further aspect thereof, the present disclosure provides a kit comprising: First component (a), comprises at least one means, and/or reagent for determining proteasome subcellular localization in at least one cell of at least one biological sample, or in any fraction of said cell. In some embodiments, the kit of the invention may optionally further comprise at least one of: (b), pre-determined calibration curve providing standard values of proteasome subcellular localization; (c), at least one control sample; and (d), instructions for use. In yet some further embodiments, the kit may further comprise at least one selective inhibitor of proteasome translocation, e.g., at least one mTOR agonist as disclosed herein.

A further aspect of the invention relates to a prognostic method for predicting and assessing responsiveness of a subject suffering from a proliferative disorder to a selective inhibitor of proteasome translocation, and optionally for monitoring disease progression. In some embodiments, the method comprising the steps of: (a) determining proteasome subcellular localization in at least one cell of at least one biological sample of the subject or in any fraction of said cell; and (b) classifying said subject as a candidate responsive subject to the selective inhibitor of proteasome translocation, if proteasome subcellular localization is cytosolic or equally distributed in at least one cell of said at least one sample. The method may optionally further comprise the step of: (c) determining proteasome subcellular localization in at least one cell of a sample of a subject classified in step (b) as a candidate responsive subject and confirming responsiveness of the subject if proteasome subcellular localization is predominantly nuclear in at least one cell contacted with the selective inhibitor of proteasome translocation.

A further aspect relates to a method for selective induction of apoptosis of cancer cells, by selective inhibition of proteasome translocation to the cytosol of said cells. The method comprising contacting the cells with an effective amount of at least one selective modulator of proteasome translocation, or with any composition comprising said selective inhibitor.

Still further aspect of the present disclosure relates to a method for treating, preventing, inhibiting, reducing, eliminating, protecting or delaying the onset of a cancer in a subject, by selectively inhibiting proteasome translocation to the cytosol of cancer cells of said subject. The method comprising the step of administering to said subject a therapeutically effective amount of at least one selective inhibitor of proteasome translocation, or with any composition comprising said selective inhibitor.

A further aspect of the present disclosure relates to a screening method for identifying at least one selective modulator of proteasome translocation. In more specific embodiments, the method comprising the steps of:

First (a), determining proteasome subcellular localization in at least one cell contacted with a candidate compound under cellular stress conditions. In some embodiments, such stress conditions may be any short-term stress conditions, for example, starvation or hypoxia.

The second step (b), involves determining the subcellular localization of at least one exported or imported control protein, in at least one cell contacted with the candidate compound under cellular stress conditions, or in any fraction of said cell. The next step (c), involves determining that the candidate compound is: (i) a selective inhibitor of proteasome translocation, if proteasome subcellular localization as determined in (a), is predominantly nuclear and the subcellular localization of the at least one exported control protein of (b), is predominantly cytosolic or equally distributed in the at least one cell contacted with said candidate compound; or (ii) a selective enhancer of proteasome translocation, if proteasome subcellular localization of (a) is predominantly cytosolic and the subcellular localization of said at least one imported control protein of (b) is predominantly nuclear in said at least one cell contacted with said candidate compound.

These and other aspects of the invention will become apparent by the hand of the following drawings.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1J: Stress-induced translocation of the 26S proteasome from the nucleus to the cytosol is active and specific

FIG. 1A. Immunofluorescence of the indicated proteasome subunits following incubation in either complete medium (Cont.), starving medium in the absence (St.), or presence of Leptomycin B (St.+LMB).

FIG. 1B. Western blot of nuclear fractions from cells treated as in FIG. 1A.

FIG. 1C. Similar to FIG. 1A but following treatment with either LMB (Cont.+LMB), or Ivermectin (Cont.+Iver.).

FIG. 1D. Immunofluorescence of fruit fly gut following feeding the flies with either complete medium (Cont.) or a solution of 5% sucrose (St.).

FIG. 1E. Western blot of nuclear fractions from cells following starvation and replenishment of a complete medium for the indicated times.

FIG. 1F. Immunofluorescence of cells following starvation and replenishment of complete medium in the presence of CHX.

FIG. 1G. Live imaging of β4-Dendra2 following starvation and replenishment for the indicated times. Fluorescence was converted from green to red at t0.

FIG. 1H. Cells were incubated for 24h at either 21% (Cont.) or 1% O2 (Hypoxia).

FIG. 1I. Cells were incubated for 8h at either 37° C. (Cont.), or 43° C. (Heat-Shock).

FIG. 1J. Cells were treated with either 2-deoxyglucose (2-DG), ionomycin (Iono.), or phenformin (Phen.).

FIG. 2A-2E: Stress-induced translocation of the 26S proteasome from the nucleus to the cytosol is active and specific FIG. 2A. U2OS cells were incubated for 8h in either complete medium (Cont.), or a medium that lacks amino acids (St.). The α6 proteasome subunit was visualized (i). Nuclear fractions (Nuclear fr.) were isolated from the corresponding cells and proteins were resolved via SDS-PAGE and blotted with the indicated antibodies (ii).

FIG. 2B. MDA-MB-231, HAP1, and MCF10A cells were incubated for 8h in either complete medium (Cont.) or a medium that lacks amino acids (St.), and the α6 proteasome subunit was imaged.

FIG. 2C. HeLa cells were incubated for 8h in a medium that lacks amino acids (St.) that was then replaced with a complete medium for additional 4h. The Rpn2 and β4 proteasome subunits were imaged. Of note is that the same cells were imaged along the entire experiment.

FIG. 2D. HeLa cells overexpressing the β4 proteasome subunit fused to the photoconvertible fluorescent protein Dendra2 were seeded on cover-slips, and fluorescence was converted from green to red, enabling further monitoring of proteins synthesized only prior to the conversion (see FIG. 1G).

FIG. 2E. HeLa cells were incubated for 8h in either complete medium (Cont.), or a medium lacking amino acids (St.). The β4 proteasome subunit was visualized via confocal microscopy by stacking images from multiple Z planes (i). Z-stacks were analyzed for quantification of basal, and stress-induced proteasome distribution between the two compartments (ii).

FIG. 3A-3I: Stress-induced proteasome translocation is mediated via a novel, non-canonical mTOR signal

FIG. 3A. Immunofluorescence of cells following treatment with Torin1.

FIG. 3B. Western blot of nuclear fractions following the indicated treatments.

FIG. 3C. Immunofluorescence of cells following silencing of mTOR.

FIG. 3D. Similar to A but following starvation or addition of the indicated amino acids.

FIG. 3E. Measurement of autophagic flux following the indicated treatment.

FIG. 3F. Western blot of cells for phosphorylated and non-phosphorylated p70-S6K following the indicated treatments.

FIG. 3G. Western blot of cells for 20 and 19S subunits following the indicated treatments.

FIG. 3H. Immunofluorescence of cells following silencing of ATF4 following the indicated conditions.

FIG. 3I. Immunofluorescence of cells following inducible expression of ATF4.

FIG. 4A-4I: Stress-induced proteasome translocation is mediated via a novel, non-canonical mTOR signal FIG. 4A. HeLa cells were infected with control shRNA (shCont.) or shRNAs targeting the uncharged-tRNA sensor GCN2 (shGCN2 #1-3). Nuclear fractions (Nuclear fr.) were isolated from the cells following 8h incubation in a complete medium (Cont.) or a medium lacking amino acids (St.). Proteins were resolved via SDS-PAGE and blotted with antibodies against the α6 proteasome subunit, Lamin A/C, and Tubulin.

FIG. 4B. Cells as in A were incubated in either complete medium (8h; Cont.), or a medium lacking amino acids for 4h (4h St.) and 8h (8h St.). The β4 subunit of the proteasome is shown.

FIG. 4C. Cells as in A were lysed following 8h incubation under the indicated conditions. Lysates were resolved via SDS-PAGE and blotted with antibodies against the indicated proteasome subunits, as well as the autophagic protein receptor LC3 (LC3-I—soluble LC3; LC3-II—autophagosome-bound lipidated form, or LC3-PE).

FIG. 4D. HeLa cells were infected with shRNAs targeting the protein kinase PIK3CA (shPIK3CA #1-3) or control shRNA (shCont.). The α6 proteasome subunit was visualized after 8h incubation in either complete medium (Cont.), or a medium lacking amino acids (St.).

FIG. 4E. HeLa cells were infected with shRNAs targeting the protein kinase AKT1 (shAKTI #1-2) or control shRNA (shCont.). The α6 proteasome subunit was monitored following 8h incubation in either complete medium (Cont.), or a medium lacking amino acids (St.).

FIG. 4F. HeLa cells were incubated for 8h in a medium lacking amino acids and the effect of added individual amino acids on the translocation of the proteasome was monitored. Single letters denote the one letter code of amino acids.

FIG. 4G. HeLa cells were incubated under the indicated conditions, and the β4 subunit of the proteasome as well as the p65 subunit of NF-κB—which is a CRM1 substrate—were visualized.

FIG. 4H. HeLa cells were incubated under the indicated conditions, and the β4 subunit of the proteasome as well as the CRM1 substrate APC (adenomatous polyposis coli) were visualized.

FIG. 4I. HeLa cells infected with GFP fused to a nuclear export signal (NES) were incubated for 8h under the indicated conditions. The GFP was visualized.

FIG. 5A-5L: Proteasome translocation is required for amino acid supplementation mediated via stimulated proteolysis, and is essential for cell survival FIG. 5A. Measurement of degradation of radiolabeled proteins in control, starved and starved cells incubated with LMB.

FIG. 5B. Measurement of degradation of the fluorogenic proteasome substrate Suc-LLVY-AMC in nuclear and cytosolic fractions in starved and control cells.

FIG. 5C. Western blot of cells (treated as indicated) for the cytosolic proteasomal substrate HMGCS1.

FIG. 5D. Western blot of extracts of cells treated under the indicated conditions and overexpressing the cytosolic protein NES-GFP-CL1.

FIG. 5E. Western blot for ubiquitin adducts of extracts of cells treated as indicated. Intensities relative to control are presented.

FIG. 5F. Live imaging of the proteasome activity probe Me4BodipyFL-Ahx3Leu3VS in cells under the different conditions.

FIG. 5G. Changes in the level of individual cellular proteins under the indicated conditions, as determined by proteomic analysis.

FIG. 5H. Changes in the levels of individual amino acids in cells incubated under the indicated conditions, as determined by metabolomics analysis.

FIG. 5I. Time course of cell survival under the indicated conditions.

FIG. 5J. Cell survival rates, relative to control, following incubation under the indicated conditions.

FIG. 5K. Immunofluorescence of cells incubated under the indicated conditions following silencing of the NPC component NUP93.

FIG. 5L. Cells as in K were treated as indicated, and survival rates were measured.

FIG. 6A-6H: Proteasome translocation is required for amino acid supplementation mediated via stimulated proteolysis, and is essential for cell survival

FIG. 6A. HeLa cells infected with cDNA coding for NES-GFP-CL1 were incubated for the indicated times in the presence of either CHX, MG132 or Chloroquine (Chq). Cells were lysed, resolved via SDS-PAGE, and blotted with an antibody against GFP.

FIG. 6B. The proteins that are most affected by the inhibition of proteasome export using LMB (uppermost 10%; FIG. 5G), were classified according to their cellular distribution—cytoplasmic, nuclear proteins, and proteins known to be shared between the two compartments.

FIG. 6C. The proteins that are most affected by the inhibition of proteasome export using YWF (uppermost 10%; FIG. 5G), were classified according to their cellular distribution.

FIG. 6D. The proteins that are most affected by the inhibition of proteasome export (uppermost 10%), were classified using Gene Ontology and KEGG pathways.

FIG. 6E. The proteins that are least affected by the inhibition of proteasome export (lowermost 10%), were classified using Gene Ontology and KEGG pathways.

FIG. 6F. Monitoring the stability of ribosomal proteins under the indicated treatments.

FIG. 6G. Survival rates of RT4 cells, relative to control, following the indicated treatments.

FIG. 6H. MDA-MB-231 cells were infected with either shRNA targeting the NPC component NUP93 or a control shRNA. Cells were further infected with GFP-NLS, and GFP localization was observed using confocal live microscopy.

FIG. 7A-7C: Stress-induced proteasomal translocation is conserved among different organs and organisms

FIG. 7A. Live imaging of the proteasome activity probe Me4BodipyFL-Ahx3Leu3VS in rat hearts perfused ex vivo under the indicated conditions.

FIG. 7B. As in FIG. 7A, but in neonatal rat neural tissue incubated under the indicated conditions (upper and lower panels—high and low magnifications, respectively).

FIG. 7C. Immunofluorescence of differentiated C2 mouse myogenic cells following incubation under the indicated treatments.

FIG. 8A-8B: Poteasome dynamics and autophagy are conjointly regulated

FIG. 8A. HeLa cells were infected with a Tet-On (TO) inducible system for the expression of either an empty vector (V0), or TFEB S142,211A. Cells were lysed following incubation in the absence or presence of Doxycycline (Dox) for 24h. Lysates were then resolved via SDS-PAGE and blotted with antibodies against TFEB and the autophagic protein receptor LC3 (LC3-I—soluble LC3; LC3-II—autophagosome-bound lipidated form, or LC3-PE).

FIG. 8B. HeLa cells were incubated for 8h in a medium lacking amino acids, and the proteasome inhibitors Bortezomib (BTZ.) or Epoxomycin (Epox.) were added for additional 2h and 4h. The β4 subunit of the proteasome was visualized.

FIG. 9A-9G: Proteasome dynamics and autophagy are conjointly regulated

FIG. 9A. Immunofluorescence of cells (i) and western blot of nuclear fractions (ii) following inducible expression of TFEB-S142,211A.

FIG. 9B. Immunofluorescence of cells following: (i) inducible expression of ZKSCAN3; (ii) incubation under the indicated conditions.

FIG. 9C. Immunofluorescence of WT or ATG5^{-/-} MEF cells following starvation.

FIG. 9D. Western blot of nuclear fractions following treatment with the proteasome inhibitor MG132 (MG).

FIG. 9E. Immunofluorescence of cells (i) and western blot of nuclear fractions (ii) following the indicated treatments. DMSO—dimethyl sulfoxide (used as a control); BTZ—Bortezomib; Lacta.—Lactacystin; Epox.—Epoxomicin.

FIG. 9F. Immunofluorescence of cells incubated under the indicated conditions.

FIG. 9G. Live imaging of the β4-GFP proteasome subunit following the indicated treatments. Dashed rectangle—MG132 was added to the cells following starvation, and live imaging was carried out at the indicated times.

FIG. 10A-10E: Aberrant cytosolic predominance of the proteasome endows multiple myeloma cells with resistance to proteasome inhibitors

FIG. 10A. Immunofluorescence of the proteasome α6 subunit in two Bortezomib-sensitive (NCI-H929 and MM.1S), and two Bortezomib-resistant (U266 and RPMI-8226) MM cells, following the indicated treatments. In the merged panels, note the visible nuclear blue staining within resistant cells, under Cont., St., and BTZ. In contrast, the nuclei of sensitive cells are masked by the reddish staining of the proteasome which largely co-localizes to the nucleus. Following YWF treatment, the proteasome is localized to the nuclei in all cell types.

FIG. 10B. Cell survival of the same cells as in FIG. 10A incubated under the indicated conditions.

FIG. 10C. Immunohistochemistry of bone marrow biopsies from MM patients, stained for the α6 proteasome subunit and for the membrane protein CD38 (a marker for MM cells).

FIG. 10D. Response of MM patients to treatment were plotted according to their proteasome distribution at the time of diagnosis.

FIG. 10L Schematic representation of relapsing MM patients who were initially sensitive to treatment with proteasome inhibitors. Presented are the response to treatment and proteasome cellular distribution in biopsies from both the 1′ diagnostic biopsy and the one taken at the time of relapse. Also shown are the intervals between the above biopsies, and the mean interval period for each patients' group.

FIG. 11: Aberrant cytosolic predominance of the proteasome to the cytosol endows multiple myeloma cells with resistance to proteasome inhibitors

Schematic representation of MM patients treated with proteasome inhibitors as first line of treatment. Presented are response to the drug and proteasome cellular distribution in the biopsy taken before initiation of treatment. For relapsed patients, same data are presented for the time of relapse.

FIG. 12A-12D: Proteasome recruitment is characteristic of stressed tumor cells in vivo, and its inhibition using YWF is cytotoxic

FIGS. 12A and 12B. Immunohistochemistry of the proteasome in Xenograft tumor sections following the indicated treatments. Periphery and core relate to the corresponding regions in the tumor.

FIG. 12C. Detection of apoptosis using TUNEL staining.

FIG. 12D. Detection of apoptosis via staining for cleaved Caspase3.

FIG. 13A-13C: Proteasome recruitment is characteristic of stressed tumor cells in vivo, and its inhibition using YWF is cytotoxic

FIGS. 13A and 13B. Immunohistochemistry of the proteasome in Xenograft tumor sections following the indicated treatments. Periphery and core relate to the corresponding regions in the tumor.

FIG. 13C. Immunohistochemistry of a tumor core section following YWF treatment, demonstrating necrotic changes that overlap area of nuclear proteasome staining.

FIG. 14A-14G: Proteasome recruitment is required for tumor growth

FIG. 14A. Tumors originating from MDA-MB-231 cells, following the indicated injected treatments, photographed for scale on a graph paper.

FIG. 14B. Plotting of tumor weights (represented under FIG. 14A) at the time of mouse sacrificing.

FIG. 14C. Tumors originating from RT4 cells, following the indicated injected treatments, photographed for scale on a graph paper.

FIG. 14D. Plotting of tumor weights (represented under FIG. 14C) at the time of mouse sacrificing.

FIG. 14E. Tumors originating from RT4 cells, following administration of the indicated amino acids in the drinking water (photographed for scale on a graph paper).

FIG. 14F. Plotting of tumor weights (represented under FIG. 14E) at the time of mouse sacrificing.

FIG. 14G. Average reduction in tumor weight (relative to control) following oral administration in the drinking water of all combinations of YWF (single, pairs and the trio) and all 20 amino acids.

FIG. 15A-15F: Proteasome recruitment is required for tumor growth

FIG. 15A. Tumors originating from RT4 cells, following the indicated treatments, photographed for scale on a graph paper. Left and right most columns are presented also under FIG. 14C.

FIG. 15B. Plotting of tumor weights at the time of mouse sacrificing. The ‘Cont. 18 d.’ and ‘YWF 18 d.’ groups are presented also under FIG. 14D.

FIG. 15C. Average reduction in tumor weight, relative to control, in the different time groups.

FIG. 15D. Plotting of tumor weights at the time of mouse sacrificing. The two left most groups are presented also under FIG. 14E.

FIG. 15E. Average reduction in tumor weight following treatment with YWF, relative to each indicated treatment.

FIG. 15F. Monitoring of tumor volume along their development, under either QLR or YWF administration via drinking water. On the last time point, the relative reduction in average volume is ˜80%, p=3.139E-05.

FIG. 16. Stress-induced proteasome translocation is prevented by D-YWF, and by mixture of the both isomers, L-YWF and D-YWF Immunofluorescence of cells incubated under stress conditions for various time points (upper panel), and with the L-isomers of YWF (L-YWF, 1.6 mM/each), or the D-isomers of YWF (D-YWF, 1.6 mM/each, or 3.2 mM/each). The lower panel shows the use of a mixture of both isomers (0.8 mM/each), under stress conditions.

FIG. 17A-17C. YWF treatment of spontaneous, endogenic tumors in mice significantly reduces tumor burden

FIG. 17A. plotting of Cecum weight of non-induced control mice (non-induced), induced mice (administered with tamoxifen) treated by placebo (control), or induced mice treated by the YWF.

FIG. 17B. plotting of the number of distinct tumors (adenomas) formed along the intestine, in induced mice treated by placebo (control), or induced mice treated by the YWF.

FIG. 17C. plotting of intestinal tumor intestinal adenomas volume in a single animal, induced mice treated with placebo (control), or induced mice treated by the YWF.

FIG. 18A-18B. YWF shrinking effect on tumors

The figure shows histochemical PROX1 staining of gut tissue sections following the indicated treatments.

FIG. 18A. shows gut tissue of a mouse treated with YWF. On the left (i) virtually all tissue is normal. On the right (ii), tissue mostly normal, with a small region of tumor cells.

FIG. 18B. shows gut tissue of a mouse treated with placebo (control). On both panels (i, ii), the tumors are too large to fit within the field of view of an x4 objective, with a small areas of normal gut tissue.

FIG. 19. Nuclear proteasome localization in YWF treated mice, correlates with inhibition of tumor growth

Figure shows immunohistochemical staining of gut tissue sections for proteasome subunit α5 following the indicated treatments (placebo (control) or the YWF). In the control group (ii), blue nuclear staining is visible due to the small amount of proteasome in nucleus. Following YWF treatment (i), the proteasome is largely sequestered within the nucleus, rendering the blue universal staining (performed as in the control group), invisible.

FIG. 20. YWF selectively affects viability of stressed cancer cells as compared to non-selective effect of 45 MmF or 45 MmW

Figure shows cell viability (% survival) of stressed (starvation) or non-stressed cancer cells treated with the indicated treatments. Control (Cont.) indicates complete medium, starvation (St.), amino acid deprived medium, Y (Tyrosine), W (Tryptophan) and F(Phenylalanine) are added in the indicated concentrations (1.6 mM for each of Y, W, F) or 45 Mm of F or W.

FIG. 21. YWF combination significantly inhibited tumor growth as compared to no effect of various high concentrations of F

The anti-tumorigenic effect of the indicated treatments was examined in vivo, using a tumor model in mice. Following tumor formation, each group was treated with the indicated treatments (YWF at 6 mM each, and various concentrations of F), and the size of tumors was compared relative to the control group (QLR). Figure shows plotting of tumor weights at the time of mouse sacrificing.

### DETAILED DESCRIPTION OF THE INVENTION

Herein, a novel layer of proteasomal regulation was identified where its compartmentalization is essential for the cell's ability to cope with stress. Following 4-8 hours of amino acids starvation, the proteasome is recruited from the nucleus to the cytosol, a process mediated via a newly identified mTOR signaling pathway. This recruitment is essential for cell survival under stress, as it provides the cell with amino acids generated by stimulated degradation of cytosolic proteins.

The inventors revealed the role of mTOR in modulating proteasome dynamics in cells. Importantly, the present disclosure demonstrates the role of proteasome dynamics as reflected by proteasome cellular localization, for example, in short term stress conditions, as well as in pathologic conditions that require cytosolic localization of the proteasome, and/or increased proteasomal activity in the cytosol. Moreover, the present disclosure provides mTOR modulators, specifically agonist/s that modulate proteasome dynamics in the cell, thereby providing an effective tool for modulating and affecting conditions and processes associated with proteasome dynamics.

Thus, a first aspect of the invention relates to an mTOR agonist comprising at least two aromatic amino acid residue or a combination of at least two aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In some specific embodiments, the mTOR agonist of the invention may comprise at least two of:

First (a), at least one tyrosine (Y) residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist may comprise in some embodiments (b), at least one tryptophan (W) residue, any mTOR agonistic tryptophan mimetic, any salt or ester thereof, any multimeric and/or polymeric form of the tryptophan residue and/or of the mTOR agonistic tryptophan mimetic, or any combination or mixture thereof. In yet some further embodiments, the mTOR agonist of the present disclosure may comprise (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof.

The present inventors revealed the role of mTOR in modulating proteasome dynamics, specifically, in stress conditions, and further provides effective mTOR agonists. The mammalian target of rapamycin (mTOR), sometimes also referred to as the mechanistic target of rapamycin and FK506-binding protein 12-rapamycin-associated protein 1 (FRAP1), is a kinase that in humans is encoded by the MTOR gene. mTOR is a member of the phosphatidylinositol 3-kinase-related kinase family of protein kinases. mTOR links with other proteins and serves as a core component of two distinct protein complexes, mTOR complex 1 and mTOR complex 2, which regulate different cellular processes. In particular, as a core component of both complexes, mTOR functions as a serine/threonine protein kinase that regulates cell growth, cell proliferation, cell motility, cell survival, protein synthesis, autophagy, and transcription. As a core component of mTORC2, mTOR also functions as a tyrosine protein kinase that promotes the activation of insulin receptors and insulin-like growth factor 1 receptors. mTORC2 is also implicated in the control and maintenance of the actin cytoskeleton. mTOR is the catalytic subunit of two structurally distinct complexes: mTORC1 and mTORC2. Both complexes localize to different subcellular compartments, thus affecting their activation and function. Upon activation by Rheb, mTORC1 localizes to the Regulator-Rag complex on the lysosome surface where it then becomes active in the presence of sufficient amino acids. mTOR Complex 1 (mTORC1) is composed of mTOR, regulatory-associated protein of mTOR (Raptor), mammalian lethal with SEC13 protein 8 (mLST8) and the non-core components PRAS40 and DEPTOR. This complex functions as a nutrient/energy/redox sensor and controls protein synthesis. The activity of mTORC1 is regulated by rapamycin, insulin, growth factors, phosphatidic acid, certain amino acids and their derivatives (e.g., 1-leucine and β-hydroxy β-methylbutyric acid), mechanical stimuli, and oxidative stress. mTOR Complex 2 (mTORC2) is composed of MTOR, rapamycin-insensitive companion of MTOR (RICTOR), MLST8, and mammalian stress-activated protein kinase interacting protein 1 (mSIN1). mTORC2 has been shown to function as an important regulator of the actin cytoskeleton through its stimulation of F-actin stress fibers, paxillin, RhoA, RacI, Cdc42, and protein kinase Cα (PKCα). mTORC2 also phosphorylates the serine/threonine protein kinase Akt/PKB, thus affecting metabolism and survival. In addition, mTORC2 exhibits tyrosine protein kinase activity and phosphorylates the insulin-like growth factor 1 receptor (IGF-IR) and insulin receptor (InsR).

As indicated above, the present disclosure provides mTOR agonists. The term “agonist”, as used herein, relates to a compound, agent or drug that activates, stimulates, increases, facilitates, enhances activation, sensitizes or up regulates the activity of a certain protein, for example the mTOR protein, to produce a biological response. According to some embodiments, wherein indicated “Increasing” or “enhancing” the mTOR activity, as used herein in connection with the mTOR agonists of the invention, it is meant that such increase or enhancement may be an increase or elevation of between about 5% to 100%, specifically, 10% to 100% of the mTOR activity. The terms “increase”, “augmentation” and “enhancement” as used herein relate to the act of becoming progressively greater in size, amount, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the activity as compared to a suitable control, e.g., mTOR activation in the absence of the modulators of the invention.

The mTOR agonists of the present disclosure affect and modulate the proteasome dynamic, for example as reflected by the cellular proteasome localization. Proteasomes, as used herein, are protein complexes which degrade unneeded or damaged proteins by proteolysis, a chemical reaction that breaks peptide bonds, mediated by proteases. Proteasomes are part of a major mechanism by which cells regulate the concentration of particular proteins and degrade misfolded proteins. Proteins are tagged for degradation with a small protein called ubiquitin. The tagging reaction is catalyzed by enzymes called ubiquitin ligases. The degradation process yields peptides of about seven to eight amino acids long, which can then be further degraded into shorter amino acid sequences and used in synthesizing new proteins. Proteasomes are found inside all eukaryotes and archaea, and in some bacteria. In structure, the proteasome is a cylindrical complex containing a “core” of four stacked rings forming a central pore. Each ring is composed of seven individual proteins. The inner two rings are made of seven β subunits that contain three to seven protease active sites. These sites are located on the interior surface of the rings, so that the target protein must enter the central pore before it is degraded. The outer two rings each contain seven α subunits whose function is to maintain a “gate” through which proteins enter the barrel. These α subunits are controlled by binding to “cap” structures or regulatory particles that recognize polyubiquitin tags attached to protein substrates and initiate the degradation process. The overall system of ubiquitination and proteasomal degradation is known as the ubiquitin-proteasome system (UPS).

The proteasome subcomponents are often referred to by their Svedberg sedimentation coefficient (denoted S). The proteasome most exclusively used in mammals is the cytosolic 26S proteasome, which is about 2000 kilodaltons (kDa) containing one 20S protein subunit (also referred to herein as the core proteasome, or CP) and two 19S regulatory cap subunits (also referred to herein as the regulatory proteasome or RP). The core is hollow and provides an enclosed cavity in which proteins are degraded. Openings at the two ends of the core allow the target protein to enter. Each end of the core particle associates with a 19S regulatory subunit that contains multiple ATPase active sites and ubiquitin binding sites. This structure recognizes polyubiquitinated proteins and transfers them to the catalytic core. An alternative form of regulatory subunit called the 11S particle may play a role in degradation of foreign peptides and can associate with the core in essentially the same manner as the 19S particle. The proteasomal degradation pathway is essential for many cellular processes, including the cell cycle, the regulation of gene expression, and responses to oxidative stress.

In some embodiments. the mTOR agonists of the invention modulate proteasome dynamics, and as such, modulate translocation and shuttling of the proteasome between the nucleus and cytosol. Proteasome dynamics as used herein is meant the transport and shuttling of the proteasome between the cytoplasm and nucleus. In some embodiments, such translocation involves dissociation into proteolytic core and regulatory complexes, and re-assembly to form the assembled proteasome. In some embodiment, the mTOR agonists of the present disclosure act in selective modulation of translocation and shuttling of the proteasome thereby resulting in nuclear or predominant nuclease localization. In some embodiments, the mTOR agonists of the present disclosure may act as selective inhibitors of translocation of the proteasome from the nucleus to the cytoplasm. In yet some alternative or additional embodiments, the mTOR agonists of the present disclosure act to enhance recruitment of the proteasome into the nucleus.

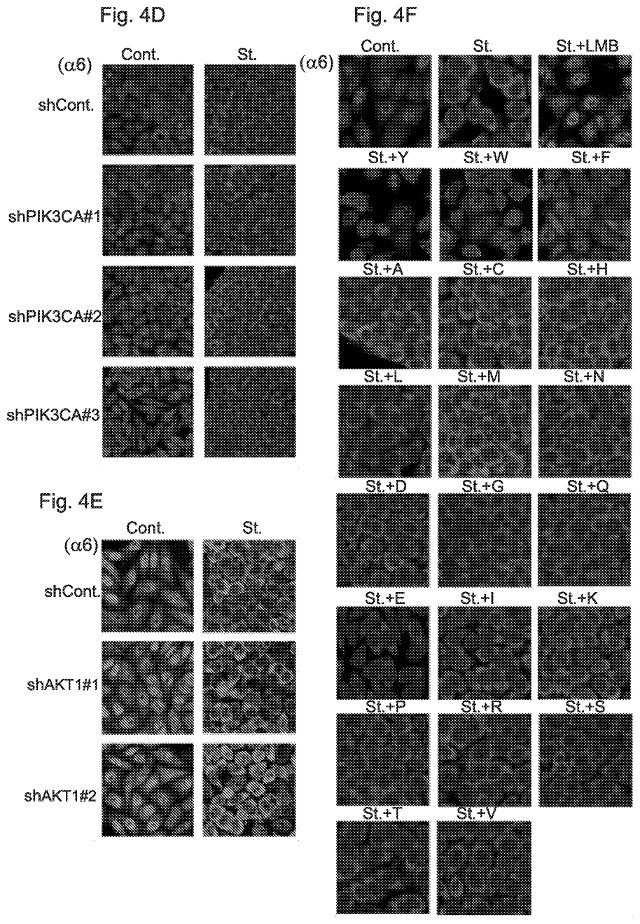

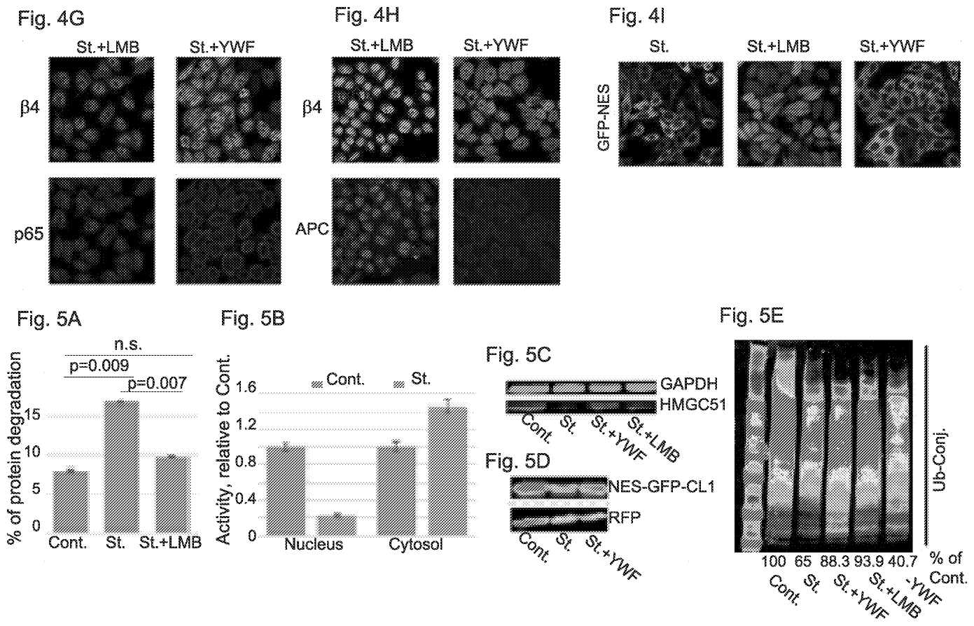

Still further, the mTOR agonists of the present disclosure act to retain, maintain or even enhance a nuclear or predominantly nuclear localization of the proteasome. A Selective modulator, as used herein is meant that the mTOR agonists of the present disclosure act exclusively, mainly, specifically, and/or predominantly, on the translocation and/or shuttling of the proteasome between the nucleus and cytoplasm, while not affecting (or almost no affecting) the translocation, export or import of other cellular elements (e.g., other substrates of exportin or importin, as shown for example by FIG. 4). In some embodiments, selective and specific modulators as indicated herein is meant that the mTOR agonists of the present disclosure selectively and exclusively act on the proteasome more than 10% to 100%, or alternatively, at least about a 2-fold to at least about a 100-fold or grater, that any modulation or effect on the translocation between nucleus-cytoplasm, of other cellular elements (e.g.. proteins, nucleic acids, etc.).

As shown by the present disclosure, a triad of aromatic amino acid residues act as mTOR agonists that modulate proteasome dynamics in short term stress conditions and may therefore be used as a nutrient sensor. An aromatic amino add (AAA) is an amino acid that includes a hydrophobic side chain, specifically, an aromatic ring. More specifically, a cyclic (ring-shaped), planar (flat) structures with a ring of resonance bonds that gives increased stability compared to other geometric or connective arrangements with the same set of atoms. An aromatic functional group or other substituent is called an aryl group. Aromatic amino acids absorb ultraviolet light at a wavelength above 250 nm and produce fluorescence. Among the 20 standard amino acids, the following are aromatic: phenylalanine, tryptophan and tyrosine.

“Aromatic amino acid” as used herein, includes natural as well as unnatural amino acids. Unnatural, aromatic amino acids comprise those that include an indole moiety in their amino acid side chain, wherein the indole ring structure can be substituted with one or more aryl group substituents. Additional examples of aromatic amino acids include but are not limited to 1-naphthylalanine, biphenylalanine, 2-napthylalananine, pentafluorophenylalanine, and 4-pyridylalanine. More specifically, the term “aromatic” as used herein, refers to a mono-, bi-, or other multi-carbocyclic, aromatic ring system. The aromatic group may optionally be fused to one or more rings chosen from aromatics, cycloalkyls, and heterocyclyls. Aromatics can have from 5-14 ring members, such as, e.g., from 5-10 ring members. One or more hydrogen atoms may also be replaced by a substituent group selected from acyl, acylamino, acyloxy, alkenyl, alkoxy, alkyl, alkynyl, amino, aromatic, aryloxy, azido, carbamoyl, carboalkoxy, carboxy, carboxyamido, carboxyamino, cyano, cycloalkyl, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, and ureido. Nonlimiting examples of aromatic groups include phenyl, naphthyl, indolyl, biphenyl, and anthracenyl.

As indicated above, in some particular embodiments, the aromatic amino acid provided by the present disclosure as effective mTOR agonist/s may be at least one of Tyrosine, Tryptophan and Phenylalanine, or any combinations thereof.

Thus, in some specific embodiments, the aromatic amino acid residue that may be provided as a selective inhibitor of proteasome translocation or as an mTOR agonist in the present disclosure is Tyrosine. Tyrosine (symbol Tyr or Y) or 4-hydroxyphenylalanine is a non-essential amino acid with a polar side group, having the formula C₉H₁₁NO₃. L-Tyrosine has the following chemical structure, as denoted by Formula VII:

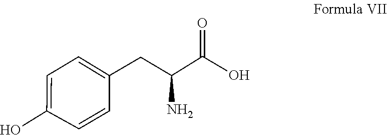 [SMILES: N[C@@H](Cc1ccc(O)cc1)C(=O)O]

While tyrosine is generally classified as a hydrophobic amino acid, it is more hydrophilic than phenylalanine. It is encoded by the codons UAC and UAU in messenger RNA (mRNA). Mammals synthesize tyrosine from the essential amino acid phenylalanine. The conversion of phe to tyr is catalyzed by the enzyme phenylalanine hydroxylase. In dopaminergic cells in the brain, tyrosine is converted to L-DOPA by the enzyme tyrosine hydroxylase (TH). TH is the rate-limiting enzyme involved in the synthesis of the neurotransmitter dopamine. Dopamine can then be converted into other catecholamines, such as norepinephrine (noradrenaline) and epinephrine (adrenaline).

The thyroid hormones triiodothyronine (T₃) and thyroxine (T₄) in the colloid of the thyroid are also derived from tyrosine.

In yet some further specific embodiments, the aromatic amino acid residue that may be provided as an mTOR agonist in the present disclosure is Tryptophan.

Tryptophan (symbol Trp or W) is an α-amino acid that is used in the biosynthesis of proteins, having the formula C₁₁H₁₂N₂O₂.

L-Tryptophan has the following chemical structure, as denoted by Formula VIII:

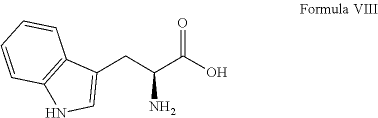 [SMILES: N[C@@H](Cc1c[nH]c2ccccc12)C(=O)O]

Tryptophan contains an α-amino group, an α-carboxylic acid group, and a side chain indole, making it a non-polar aromatic amino acid. It is encoded by the codon UGG. Like other amino acids, tryptophan is a zwitterion at physiological pH where the amino group is protonated (—NH₃⁺; pKₐ=9.39) and the carboxylic acid is deprotonated (—COO⁻; pKₐ=2.38).

Tryptophan functions as a biochemical precursor for the following compounds: Serotonin (a neurotransmitter), synthesized by tryptophan hydroxylase; Melatonin (a neurohormone) is in turn synthesized from serotonin, via N-acetyltransferase and 5-hydroxyindole-O-methyltransferase enzymes; Niacin, also known as vitamin B3, is synthesized from tryptophan via kynurenine and quinolinic acids; Auxins (a class of phytohormones) are synthesized from tryptophan. Tryptophan is also a precursor to the neurotransmitter serotonin, the hormone melatonin and vitamin B3.

Still further, in some specific embodiments, the aromatic amino acid that may be provided as an mTOR agonist in the methods of the present disclosure is Phenylalanine.

Phenylalanine (symbol Phe or F) is an essential α-amino acid with the formula C₉H₁₁NO₂. It can be viewed as a benzyl group substituted for the methyl group of alanine, or a phenyl group in place of a terminal hydrogen of alanine.

L-Phenylalanine has the following chemical structure, as denoted by Formula IX:

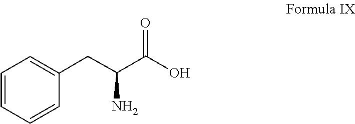 [SMILES: N[C@@H](Cc1ccccc1)C(=O)O]

This essential amino acid is classified as neutral, and nonpolar because of the inert and hydrophobic nature of the benzyl side chain. The L-isomer is used to biochemically form proteins, coded for by DNA. Phenylalanine is a precursor for tyrosine, the monoamine neurotransmitters dopamine, norepinephrine (noradrenaline), and epinephrine (adrenaline), and the skin pigment melanin. It is encoded by the codons UUU and UUC.

It should be noted that phenylalanine and tryptophan are essential amino acids. Essential amino acids, for example, phenylalanine and tryptophan, are amino acid residues that are not synthesized de novo in humans and other animals, and therefore must be provided by an external source.

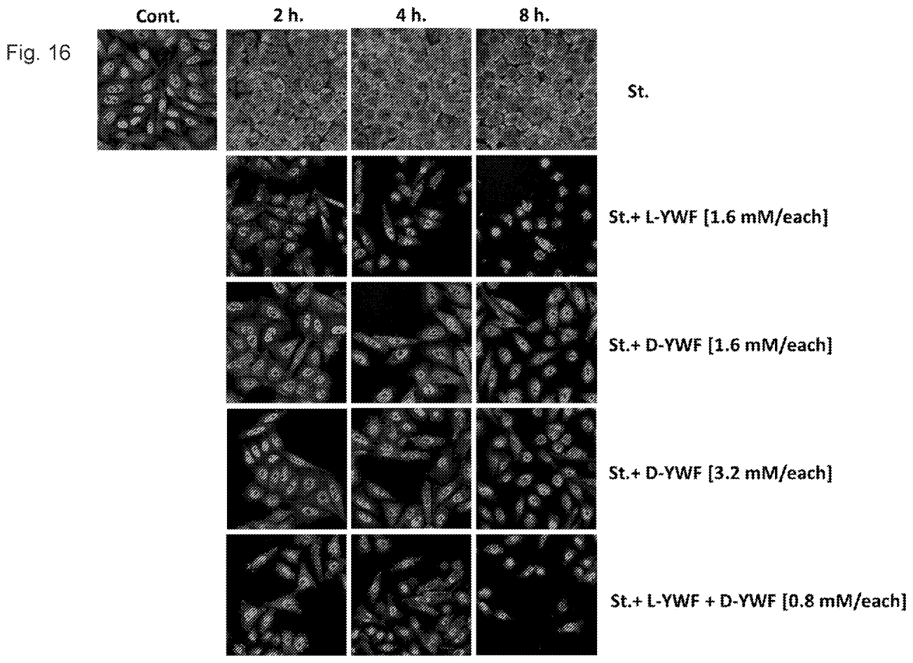

The mTOR agonist/s of the present disclosure comprise at least one of tyrosine, tryptophan and/or phenylalanine, that are interchangeably referred to herein as “tyrosine, tryptophan and/or phenylalanine”, “Tyr, Trp and/or Phe”, “Y, W and/or F”, or “YWF”. It should be noted that every amino acid (except glycine) can occur in two isomeric forms, because of the possibility of forming two different enantiomers (stereoisomers) around the central carbon atom. By convention, these are called L- and D-forms, analogous to left-handed and right-handed configurations. The amino acid residues used in the agonists of the invention can be in D-configuration or L-configuration (referred to herein as D- or L-enantiomers). In yet some further embodiments, the aromatic amino acids of the mTOR agonists of present disclosure may comprise at least one amino acid residue in the D-form. As shown by FIG. 16, the L-form of the YWF triad, as well as the D-form of the YWF, effectively inhibited proteasome translocation to the cytosol. Moreover, the racemic mixture of both, D-isomers of YWF and L-isomers of YWF, efficiently inhibited proteasome recruitment to the cytosol.

More specifically, as shown by Formula VII, VIII and IX, the above-described aromatic amino acids i.e., Tyrosine, Tryptophan and Phenylalanine, possess all a general structure comprising a core structure of 2-aminopropionic acid (alanine) wherein the beta carbon of such structure is substituted with an optionally substituted aryl. In some embodiment, the agonist/s of the invention must display at least one benzene ring and an Alanine equivalent structure.

In some embodiments, the optionally substituted aryl is a phenolic group wherein the beta carbon of the core structure is connected to such group in a para position relative to the hydroxyl of the phenolic group. Particular embodiments for such structure, may comprise tyrosine.

In some other embodiments, the aryl is a benzene ring. Particular embodiments for such structure, may comprise phenylalanine.

In yet some other embodiments, the aryl is indolyl which is connected to the beta carbon of the core structure via C₃ of the indolic substituent. Particular embodiments for such structure, may comprise tryptophan.

Still further, the disclosure contemplates the use of any at least one Y mimetic, at least one W mimetic, or at least one F mimetic which is capable of agonistic mTOR alone, or in combination, as measured by proteasome nuclear localization. “Amino acid mimedcs”, as used herein, refers to chemical compounds having a structure that is different from the general chemical structure of an amino acid, but that functions in a manner similar to a naturally occurring amino acid.

As used herein “tyrosine mimetic” and “Y mimetic”, “tryptophan mimetic” and “W mimetic” and “phenylalanine mimetic” and “F mimetic”, are used interchangeably to refer to any agent that either emulates the biological effects of tyrosine, tryptophan and/or phenylalanine, on mTOR activation in a cell, as measured by proteasome nuclear localization in response to the agonists of the present disclosure, or to any agent that increases, directly or indirectly, the level, and/or bio availability and/or stability of at least one of tyrosine, tryptophan and/or phenylalanine in a cell. The Y, W and/or F mimetic can be any kind of agent. Exemplary Y, W and/or F mimetics include, but are not limited to, small organic or inorganic molecules; L-tyrosine, L-tryptophan and/or L-phenylalanine, D-tyrosine, D-tryptophan and/or D-phenylalanine or any combinations thereof, an mTOR agonistic tyrosine, tryptophan and/or phenylalanine mimetic, saccharides, oligosaccharides, polysaccharides, a biological macromolecule that may be any one of peptides, non-standard peptides, polypeptides, non-standard polypeptides, proteins, non-standard proteins, peptide analogs and derivatives enriched for L-tyrosine, L-tryptophan and/or L-phenylalanine and/or mTOR agonistic tyrosine, tryptophan and/or phenylalanine mimetics, peptidomimetics, nucleic acids such as siRNAs, shRNAs, antisense RNAs, ribozymes, and aptamers that directly or indirectly alter the levels of at least one of Y, W, F, an extract made from biological materials selected from the group consisting of bacteria, plants, fungi, animal cells, and animal tissues; naturally occurring or synthetic compositions; and any combination thereof.

The disclosure further contemplates methods of identifying tyrosine, tryptophan and/or phenylalanine mimetics, for example by assessing the ability of a candidate agent to emulate the biological effects of tyrosine, tryptophan and/or phenylalanine on a selective inhibition of proteasome translocation or mTOR activation in a cell, that results in an increase in the nuclear localization of the proteasome. In some embodiments, methods of identifying tyrosine, tryptophan and/or phenylalanine mimetics include assessing the ability of a candidate agent to emulate the biological effects of tyrosine for example, when tyrosine is used in combination with tryptophan and phenylalanine to simulate a selective inhibition of proteasome translocation or mTOR activation, and thereby proteasome nuclear localization in a cell.

The term “mTOR agonistic tyrosine, tryptophan and/or phenylalanine mimetic” as used herein means a mimetic of tyrosine, tryptophan and/or phenylalanine which, when administered to a subject alone (in the form of a single compound or as part of a non-standard peptide, non-standard polypeptide, or non-standard protein, enriched for such mimetic) or in combination with the other components utilized in the present disclosure causes an increase in mTOR activity and proteasome cellular localization, and thereby to an increase in proteasome nuclear localization in one or more cells and/or tissues or cells of that subject. as compared to the mTOR activity prior to administration of the mimetic. It should be noted that any methods and means may be used for determining the cellular localization of the proteasome. In some embodiments, any of the methods disclosed by the preset disclosure in connection with other aspects of the invention, are also applicable for the present aspect as well. In some embodiments, the subject is determined to be deficient in tyrosine, tryptophan and/or phenylalanine prior to administration. In some embodiments, an mTOR agonistic tyrosine, tryptophan and/or phenylalanine mimetic causes an increase in mTOR activity and thereby proteasome nuclear localization that is between 50% and 500% of the increase caused by administering an equimolar amount of L-tyrosine. L-tryptophan and/or L-phenylalanine and/or D-tyrosine, D-tryptophan and/or D-phenylalanine, and any combinations thereof. In some embodiments, an mTOR agonistic tyrosine, tryptophan and/or phenylalanine mimetic causes an increase in mTOR activity and thereby proteasome nuclear localization, that is between 80%> and 120% of the increase caused by administering an equimolar amount of L-tyrosine, tryptophan and/or phenylalanine. In some embodiments, an mTOR agonistic tyrosine, tryptophan and/or phenylalanine mimetic causes a selective inhibition of proteasome translocation and/or an increase in mTOR activity, and thereby proteasome nuclear localization, that is equal to or greater than the increase caused by administering an equimolar amount of L-tyrosine, L-tryptophan and/or L-phenylalanine.

In some embodiments, the Y, W and/or F mimetic is not the native amino acid tyrosine, tryptophan and/or phenylalanine. In some embodiments, the Y, W and/or F mimetic is not a naturally occurring source of tyrosine, tryptophan and/or phenylalanine. In some embodiments, the Y, W and/or F mimetic are not a dietary source of tyrosine, tryptophan and/or phenylalanine.

In some embodiments, the Y, W and/or F mimetic comprise the native amino acid tyrosine, tryptophan and/or phenylalanine. As used herein, “native amino acid” refers to the L-form of the amino acid which naturally occurs in proteins; thus, the term “native amino acid tyrosine, tryptophan and/or phenylalanine” refers to L-tyrosine, L-tryptophan and/or L-phenylalanine. In some embodiments, the native amino acid tyrosine, tryptophan and/or phenylalanine is isolated and/or purified. In some embodiments, the amino acid residues can be in D-configuration or L-configuration (referred to herein as D- or L-enantiomers).

In some embodiments, the Y, W and/or F mimetic comprises the native amino acid tyrosine, tryptophan and/or phenylalanine (Y, W and/or F). In some embodiments, the native amino acid tyrosine, tryptophan and/or phenylalanine is isolated and/or purified.

In some embodiments, the Y, W and/or F mimetic comprises a polypeptide comprising the native amino acid tyrosine, tryptophan and/or phenylalanine or any mixture of native and non-native YWF. In some embodiments, the Y, W and/or F mimetic comprises a polypeptide comprising a derivative of the native amino acid tyrosine, tryptophan and/or phenylalanine. In some embodiments, the Y. W and/or F mimetic comprises a polypeptide comprising an analog of the native amino acid tyrosine, tryptophan and/or phenylalanine. In some embodiments, the Y, W and/or F mimetic comprises a polypeptide comprising a combination of the native amino acid tyrosine, tryptophan and/or phenylalanine, a derivative of the native amino acid tyrosine, tryptophan and/or phenylalanine and/or an analog of the native amino acid tyrosine, tryptophan and/or phenylalanine.

In some embodiments, the multimeric and/or polymeric form of the aromatic amino acid resides provided in the mTOR agonist of the invention further encompass any peptide, non-standard peptide, polypeptide, non-standard polypeptide, protein or non-standard protein any of which is enriched for one, two, or all three aromatic amino acid residues or mimetics thereof, specifically, at least one of Y, W and/or F (tyrosine, tryptophan and/or phenylalanine), and/or mTOR agonistic mimetic thereof.

As indicated herein, in some embodiments, the aromatic amino acid residues of the invention may be provided in, or as a polypeptide. A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Exemplary nonstandard amino acids include, e.g., singly or multiply halogenated (e.g., tluorinated) amino acids, D-amino acids, homo-ammo acids, N-alkyl amino acids (other than proline), dehydroamino acids, aromatic amino acids (other than histidine, phenylalanine, tyrosine and tryptophan), and α,α disubstituted amino acids, An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides. One of ordinary skill in the art will understand that a protein may be composed of a single amino acid chain or multiple chains associated covalently or noncovalently.

More specifically, the polypeptide comprising the native amino acid tyrosine, tryptophan and/or phenylalanine (and/or analogs and/or derivatives of the native amino acid tyrosine, tryptophan and/or phenylalanine) can be of any length, specifically, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 4, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 250, 500, 1000, or more residues. In some embodiments, the polypeptide comprising tyrosine, tryptophan and/or phenylalanine consists entirely of tyrosine, tryptophan and/or phenylalanine residues. In some embodiments, the polypeptide comprising the native amino acid tyrosine, tryptophan and/or phenylalanine is polypeptide enriched for tyrosine, tryptophan and/or phenylalanine residues. In some embodiments, the polypeptide enriched for tyrosine, tryptophan and/or phenylalanine residues comprises at least 10% content of tyrosine, tryptophan and/or phenylalanine residues relative to other amino acid residues. In some embodiments, the polypeptide enriched for tyrosine, tryptophan and/or phenylalanine residues comprises at least 12%, at least 15%, at least 22%, at least 25%, at least 31%, at least 35%, at least 40%, at least 44%, at least 47%, at least 50%, at least 53%, at least 58%, at least 61%, at least 66%. at least 70%, at least 75%. or more content of tyrosine, tryptophan and/or phenylalanine residues. In some embodiments, the polypeptide enriched for tyrosine, tryptophan and/or phenylalanine residues comprises at least 80%, at least 85%, at least 90%, at least 91%, at least 92%, at least 93%, at least 94%, at least 95%, at least 96%, at least 97%, at least 98%, at least 99% content of tyrosine, tryptophan and/or phenylalanine residues. In yet some further embodiments, the selective modulator of proteasome shuttling, translocation, that also acts as an mTOR agonist in accordance with the present disclosure, may comprise two or more polypeptides each is enriched for at least one of Y, W, F, as discussed above.

In certain exemplary embodiments, disclosed herein is a synthetic oligopeptide, peptide, or polypeptide comprising YWF residues. Such synthetic YWF oligopeptides, peptides, and polypeptides can be of any length (e.g., 2-20 residues, 20-100 residues, 100-1,000 residues, 500-2,000 residues, 1,000-10,000 residues, or longer). The residues comprising such YWF oligopeptides, peptides, or polypeptides can ordered in any fashion, e.g., YWF, YFW, WFY, WYF, FYW, FWY. The residues comprising such YWF oligopeptides, peptides, or polypeptides can also be structured as repeats ordered in any fashion, such as YYY repeats, WWW repeats. FFF repeats, YWF repeats, in certain embodiments, the synthetic YWF oligopeptides, peptides, and polypeptides contains at least 20%, 30%, at least 35%, at least 40%, at least 45%, at least 50%. at least 60%, at least 70%, at least 80%, at least 90%, at least 95%, at least 97% or more, and even 100% YWF content. In some embodiments, the synthetic YWF oligopeptides, peptides, and polypeptides contain at least 10% YWF content. In some embodiments, the synthetic YWF oligopeptides, peptides, and polypeptides contain at least 15% YWF content. In some embodiments, the synthetic YWF oligopeptides, peptides, and polypeptides contain at least 20% YWF content. In some embodiments, the synthetic YWF oligopeptides, peptides, and polypeptides contain at least 25% YWF content. In some embodiments, the synthetic YWF oligopeptides, peptides, and polypeptides contain at least 30% YWF content. In some embodiments, the synthetic YWF oligopeptides, peptides, and polypeptides contain at least 35% YWF content. In some embodiments, the synthetic YWF oligopeptides, peptides, and polypeptides contain at least 40% YWF content. In some embodiments, the synthetic YWF oligopeptides, peptides, and polypeptides contain at least 45% YWF content. In some embodiments, the synthetic YWF oligopeptides, peptides, and polypeptides contain at least 50% YWF content. In some embodiments, the synthetic YWF oligopeptides, peptides, and polypeptides contain at least 55% YWF content. In some embodiments, the synthetic YWF oligopeptides, peptides, and polypeptides contain at least 60% YWF content. In some embodiments, the synthetic YWF oligopeptides, peptides, and polypeptides contain at least 65% YWF content. In some embodiments, the synthetic YWF oligopeptides, peptides, and polypeptides contain at least 70% YWF content. In some embodiments, the synthetic YWF oligopeptides, peptides, and polypeptides contain at least 75% YWF content. In some embodiments, the synthetic YWF oligopeptides, peptides, and polypeptides contain at least 80% YWF content. In some embodiments, the synthetic YWF oligopeptides, peptides, and polypeptides contain at least 85% YWF content. In some embodiments, the synthetic YWF oligopeptides, peptides, and polypeptides contain at least 90% YWF content. In some embodiments, the synthetic YWF oligopeptides, peptides, and polypeptides contain at least 95% YWF content. In some embodiments, the synthetic YWF oligopeptides, peptides, and polypeptides contain 100% YWF content.

In some embodiments, the polypeptide comprising tyrosine, tryptophan and/or phenylalanine is enriched for tyrosine, tryptophan and/or phenylalanine residues. In some embodiments, the polypeptide enriched for tyrosine, tryptophan and/or phenylalanine comprises a tyrosine, tryptophan and/or phenylalanine-rich repeat containing protein or a fragment thereof. Those skilled in the art will appreciate that a variety of methods exist for obtaining polypeptide comprising and/or enriched for tyrosine, tryptophan and/or phenylalanine, including, for example, isolating tyrosine, tryptophan and/or phenylalanine-rich repeats or fragments from polypeptide enriched for tyrosine, tryptophan and/or phenylalanine, synthetic routes, and recombinant methods (e.g., in vitro transcription and/or translation of nucleic acids comprising tyrosine, tryptophan and/or phenylalanine codons UAU, UAC (Tyr), UGG (Trp), UUU, UUC (Phe). Recombinant methods of producing a peptide through the introduction of a vector including nucleic acid encoding the peptide into a suitable host cell is well known in the art, such as is described in Sambrook et al, Molecular Cloning: A Laboratory Manual, 2d Ed. Vols 1 to 8, Cold Spring Harbor, NY (1989); M. W. Pennington and B. M. Dunn, Methods in Molecular Biology: Peptide Synthesis Protocols, Vol 35, Hurnana Press, Totawa, NJ. Peptides can also be chemically synthesized using methods well known in the art.

In some embodiments, a polypeptide comprising tyrosine, tryptophan and/or phenylalanine or enriched for tyrosine, tryptophan and/or phenylalanine is not a dietary source of tyrosine, tryptophan and/or phenylalanine. As used herein, “dietary source of tyrosine, tryptophan and/or phenylalanine” refers to a source of tyrosine, tryptophan and/or phenylalanine in which, prior to ingestion, chewing, or digestion, the tyrosine, tryptophan and/or phenylalanine is found in its natural state as part of an intact polypeptide within the source (e.g., meats (e.g., chicken, beef, etc.), legumes, grains, vegetables, dairy products (e.g., milk, cheese), eggs, nuts, seeds, seafood, etc.).

In some embodiments, a polypeptide comprising tyrosine, tryptophan and/or phenylalanine or enriched for tyrosine, tryptophan and/or phenylalanine does not include any non-essential amino acids other than tyrosine. In some embodiments, a polypeptide comprising tyrosine, tryptophan and/or phenylalanine or enriched for tyrosine, tryptophan and/or phenylalanine does not include any essential amino acids other than tryptophan and phenylalanine. In some embodiments, a polypeptide comprising tyrosine, tryptophan and/or phenylalanine or enriched for tyrosine, tryptophan and/or phenylalanine includes at least one non-native form of the amino acid tyrosine, tryptophan and/or phenylalanine.

In some embodiments, the Y, W and/or F mimetic comprises a derivative of the native amino acid tyrosine, tryptophan and/or phenylalanine. It is contemplated that any derivative of Y, W and/or F which activates mTOR and lead to proteasome nuclear localization, can be used. Y, W, and/or F derivatives which activate mTOR activation and proteasome nuclear localization can be readily determined by the skilled artisan according to the teachings disclosed herein (e.g., assaying for Y, W, and/or F derivatives which increase proteasome nuclear localization either alone, or in combination with the amino acids tyrosine, tryptophan and phenylalanine or mimetics of tyrosine, tryptophan or phenylalanine).

In some embodiments, the derivative of Y, W, and/or F comprises a C-terminus modification to Y, W, and/or F. As used herein, a “C-terminus modification” refers to the addition of a moiety or substituent group to the amino acid via a linkage between the carboxylic acid group of the amino acid and the moiety or substituent group to be added to the amino acid. The disclosure contemplates any C-terminus modification to Y, W, and/or F in which Y, W, and/or F retains the ability to stimulate mTOR activation and thereby leading to proteasome nuclear localization, when used alone, or in combination with any of the aromatic amino acids tyrosine, tryptophan and phenylalanine, as measured by proteasome nuclear localization. In some embodiments, the C-terminus modification to Y, W, and/or F comprises a carboxy alkyl of Y, W, and/or F. In some embodiments, the C-terminus modification to Y, W, and/or F comprises a carboxy alky ester of Y, W. and/or F. In some embodiments, the C-terminus modification to Y, W. and/or F comprises a carboxy alkyl ester. As used herein, the term “alkyl” refers to saturated non-aromatic hydrocarbon chain that may be a straight chain or branched chain, containing the indicated number of carbon atoms (these include without limitation methyl. ethyl, propyl, allyl, or propargyl), which may be optionally inserted with N, O, S, SS, SO₂, C(O), C(O)O, OC(O), C(O)N or NC(O). For example, C i-Ce indicates that the group may have from 1 to 6 (inclusive) carbon atoms in it. In some embodiments, the C-terminus modification to L comprises a carboxy alkenyl ester. As used herein, the term “alkenyl” refers to an alkyl that comprises at least one double bond. Exemplary alkenyl groups include, but are not limited to, for example, ethenyl, propenyl, butenyl, 1-methyl-2-buten-1-yl and the like. In some embodiments, the C-terminus modification to Y, W, F comprises a carboxy alkynyl ester. As used herein, the term “alkynyl” refers to an alkyl that comprises at least one triple bond. In some embodiments, the carboxy ester comprises tyrosine, tryptophan and/or phenylalanine carboxy methyl ester. In some embodiments, the carboxy ester comprises tyrosine, tryptophan and/or phenylalanine carboxy ethyl ester.

In some embodiments, derivative of Y, W and/or F comprises an N-terminus modification to Y, W and/or F. As used herein, “N-terminus modification” refers to the addition of a moiety or substituent group to the amino acid via a linkage between the alpha amino group of the amino acid and the moiety or substituent group to be added to the amino acid. The disclosure contemplates any N-terminus modification to Y, W and/or F in which the N-terminus modified Y, W and/or F retains the ability to stimulate mTOR activation and thereby, proteasome nuclear localization either alone, or in combination with the amino acids tyrosine, tryptophan and phenylalanine, as measured by proteasome nuclear localization.

In some embodiments, the derivative of Y, W, and/or F comprises Y, W, and/or F modified by an amino bulky substituent group. As used herein “amino bulky substituent group” refers to a bulky substituent group which is linked to the amino acid via the alpha amino group. The disclosure contemplates the use of any Y, W, and/or F derivative comprising an amino bulky substituent group that retains its ability to stimulate mTOR activation and thereby, proteasome nuclear localization, when used alone, or in combination with the amino acid residues tryptophan and phenylalanine, as measured by proteasome nuclear localization. An exemplary amino bulky substituent group is a carboxybenzyl (Cbz) protecting group. Accordingly, in some embodiments, the derivative of Y, W, and/or F comprises Y, W, and/or F modified by an amino carboxybenzyl (Cbz) protecting group. Other suitable amino bulky substituent groups are apparent to those skilled in the art.

In some embodiments, the derivative of Y, W and/or F comprises a side-chain modification to Y, W and/or F. As used herein “side-chain modification” refers to the addition of a moiety or substituent group to the side-chain of the amino acid via a linkage (e.g., covalent bond) between the side-chain and the moiety or chemical group to be added. The disclosure contemplates the use of any side-chain modification that permits the side-chain modified amino acid to retain its ability to stimulate mTOR activation when used alone, or in combination with any one of the amino acids tyrosine, tryptophan and phenylalanine or mimetics thereof, as measured by proteasome nuclear localization. An exemplary side-chain modification is a diazirine modification. Accordingly, in some embodiments, the Y, W and/or F derivative comprises a photo-crosslinkable Y, W, and/or F with a diazirine-modified side chain. In some embodiments, the derivative of Y, W, and/or F comprises an unnatural amino acid. In some embodiments, the derivative of Y, W, and/or F comprises a salt of Y, W, and/or F. In some embodiments, the derivative of Y, W, and/or F comprises a nitrate of Y, W, and/or F. In some embodiments, the derivative of Y, W, and/or F comprises a nitrite of Y, W, and/or F. In some embodiments, the Y, W, and/or F mimetic comprises an analog of the native amino acid tyrosine, tryptophan and/or phenylalanine. It is contemplated that any analog of Y, W, and/or F which stimulates mTOR activation when used alone, or in combination with the amino acid tryptophan and phenylalanine, as measured by proteasome nuclear localization can be used. Y, W, and/or F analogs which stimulate mTOR activation can be readily determined by the skilled artisan according to the teachings disclosed herein (e.g., assaying for Y, W, and/or F analogs which increase proteasome nuclear localization). It should be understood, that the present disclosure further encompasses in some particular and non-limiting embodiments thereof, any Deuterated, Fluorinated, Acetylated or Methylated forms of any one of the L- or D-tyrosine, the L- or D-phenylalanine or L- or D-tryptophan. More specifically, deuterium-substituted amino acids (deuterated amino acids) applicable as analogs of the present invention may include but are not limited to L-Tyrosine-(phenyl-3,5-d₂), L-4-Hydroxyphenyl-2,3,5,6-d₄-alanin and L-Tryptophan-(indole-ds). Methylated aromatic amino acids residues include but are not limited to any one of L-Tyrosine methyl ester, O-Methyl-L-tyrosine, α-Methyl-L-tyrosine, α-Methyl-DL-tyrosine methyl ester hydrochloride, α-Methyl-L-tyrosine, α-Methyl-DL-tyrosine, α-Methyl-DL-tryptophan, O-Methyl-L-tyrosine, N-Methyl-phenethylamine, β-Methylphenethylamine, N, N-Dimethylphenethylamine, 3-Methylphenethylamine, (R)-(+)-p-Methylphenethylamine, N-Methyl-N-(1-phenylethyl)amine, 2-methylphenethylamine, 4-Bromo-N-methylbenzylamine, 3-Bromo-N-methylbenzylamine, (S)-p-Methylphenethylamine, p-Chloro-p-methylphenethylamine hydrochl, α-Methyl-DL-tryptophan, L-Tryptophan methyl ester hydrochloride, D-Tryptophan methyl ester hydrochloride, L-Tryptophan ethyl ester hydrochloride, L-Tryptophan benzyl ester, L-Tyrosine methyl ester hydrochloride, L-Phenylalanine methyl ester hydrochlori, DL-tryptophan methyl ester, N-acetyl-1-tryptophan methyl ester. Still further, Fluorinated tyrosine, phenylalanine or tryptophan include but are not limited to any one of 5-Fluoro-L-tryptophan, 5-Fluoro-DL-tryptophan, 4-Fluoro-DL-tryptophan, 6-Fluoro-L-Tryptophan, 5-Methyl-DL-tryptophan, 5-Bromo-DL-tryptophan, 7-Azatryptophan, m-Fluoro-DL-tyrosine, p-Fluoro-L-phenylalanine, o-Fluoro-DL-phenylalanine, p-Fluoro-DL-phenylalanine, 4-Chloro-DL-phenylalanine, m-Fluoro-L-phenylalanine, 3-Nitro-L-tyrosine. In some further embodiments of the present disclosure Acetylated aromatic amino acids residues include but are not limited to any one of N-acetyl-L-tyrosine, N-Acetyl-L-phenylalanine, L-Phenylalanine methyl ester hydrochloride, N-Acetyl-D-phenylalanine, N-Acetyl-L-tryptophan.

Exemplary analogs of tyrosine and/or phenylalanine that may be applicable in accordance with the present disclosure include but are not limited to any one of (2R, 3S)/(2S, 3R)-Racemic Fmoc-β-hydroxyphenylalanine, Boc-2-cyano-L-phenylalanine, Boc-L-thyroxine, Boc-O-methyl L-tyrosine, Fmoc-β-methyl-DL-phenylalanine, Fmoc-2-cyano-L-phenylalanine, Fmoc 3,4-dichloro-L-phenylalanine, Fmoc-3,4-difluoro-L-phenylalanine, Fmoc-3,4-dihydroxy-L-phenylalanine, Fmoc-3,4-dihydroxy-phenylalanine, acetonide protected, Fmoc 3-amino-L-tyrosine, Fmoc-3-chloro-L-tyrosine, Fmoc-3-fluoro-DL-tyrosine, Fmoc 3-nitro-L-tyrosine, Fmoc-4-(Boc-amino)-L-phenylalanine, Fmoc-4-(Boc-aminomethyl)-L-phenylalanine, Fmoc-4-(phosphonomethyl)-phenylalanine, Fmoc-4-(phosphonomethyl)-phenylalanine, Fmoc-4-benzoyl-D-phenylalanine. Still further, in some embodiments, exemplary analogs of tryptophan that may be applicable in accordance with the present disclosure include but are not limited to any one of Boc-4-methyl-DL-tryptophan, Boc-4-methyl-DL-tryptophan, Boc-6-fluoro-DL-tryptophan, Boc-6-methyl-DL-tryptophan, Boc-DL-7-azatryptophan, Fmoc-(R)-7-Azatryptophan, Fmoc-5-benzyloxy DL-tryptophan, Fmoc-5-bromo-DL-tryptophan, Fmoc-5-chloro-DL-tryptophan, Fmoc 5-fluoro-DL-tryptophan, Fmoc-5-fluoro-DL-tryptophan, Fmoc-5-hydroxy-L-tryptophan, Fmoc-5-hydroxy-L-tryptophan, Fmoc-5-methoxy-L-tryptophan, Fmoc-5-methoxy-L-tryptophan, Fmoc-6-chloro-L-tryptophan, Fmoc-6-methyl-DL-tryptophan, Fmoc-7-methyl-DL-tryptophan, Fmoc-DL-7-azatryptophan.

In some embodiments, the Y, W, and/or F mimetic comprises a metabolite of the native amino acid tyrosine. It is further contemplated that any metabolite of tyrosine that stimulates mTOR activation alone or in combination with the amino acid residues tryptophan and phenylalanine or mimetics thereof can be used. Y, W. and/or F derivatives which stimulate mTOR activation can be readily determined by the skilled artisan according to the teachings disclosed herein (e.g., assaying for metabolites of Y, W, and/or F which increase proteasome nuclear localization when used alone, or in combination with tryptophan and phenylalanine or mimetics thereof.

It should be appreciated that the present disclosure provides the aromatic amino acid residues, specifically, tyrosine, tryptophan and/or phenylalanine and/or any serogates thereof, any salt, base, ester or amide thereof, any enantiomer, stereoisomer or disterioisomer thereof, or any combination or mixture thereof. Pharmaceutically acceptable salts include salts of acidic or basic groups present in compounds, specifically, the aromatic amino acid residues of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain aromatic amino acid residues of the present disclosure can form pharmaceutically acceptable salts. Suitable base salts include, but are not limited to, aluminum, calcium, lithium, magnesium, potassium, sodium, zinc, and diethanolamine salts.

The present disclosure provides effective mTOR agonist/s that may comprise either one aromatic amino acid residue, for example, any one of tyrosine, tryptophan and/or phenylalanine or any mimetics thereof, or any combination of at least two of tyrosine, tryptophan and/or phenylalanine and/or mimetics thereof. As such, the present disclosure further provides combinations, specifically combinations comprising at least two of tyrosine, tryptophan and/or phenylalanine, and/or any mimetics or derivatives thereof. In some embodiments, the effective amount of the at least one mTOR agonist/s in the combination of the present disclosure is sufficient for modulating proteasome dynamics in at least one cell.

In some embodiments, the selective inhibitor of proteasome translocation, and/or mTOR agonist in accordance with the invention may comprise at least one tyrosine residue, any mimetic, any salt or ester thereof, any multimeric and/or polymeric form thereof, and any combinations or mixtures thereof, and at least one tryptophane residue, any mimetic, any salt or ester thereof, any multimeric and/or polymeric form thereof, and any combinations or mixtures thereof. In some further embodiments, the mTOR agonist in accordance with the invention may comprise at least one tyrosine residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. In yet some further embodiments, the mTOR agonist in accordance with the invention may comprise at least one tryptophane residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof.

In some particular embodiments, the mTOR agonist of the present disclosure may comprise the following three components: first component (a), comprises at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist of the invention further comprises component (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof. The mTOR agonist of the invention further comprises component (c), phenylalanine residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof. It should be understood that the aromatic amino acid residues of the mTOR agonists of the present disclose or any mimetics thereof, may be presented in a mixture of all three YWF, at any appropriate quantitative ratio. The quantitative ratio used may be for example, 1:1:1, 1:2:3, 1:10:100, 1:10:100:1000 etc, or any one of 1-10⁶:1-10⁶:1-10⁶. In some embodiments the quantitative ratio may be any one of 1:1:1 1:1:2, 1:1:3, 1:1, 1:1:5, 1:1:6, 1:1:7, 1:1:8, 1:3, 1:4, 1:5, 1:6, 1:7, 1:8, 1:9, 1:10, 1:2:1, 1:3:1, 1:4:1, 1:5:1, 1:6:1, 1:7:1, 1:8:1, 1:9:1, 1:10:1, 2:1:1, 3:1:1, 4:1:1, 5:1:1, 6:1:1, 7:1:1, 8:1:1, 9:1:1, 10:1:1, or any other suitable ratio of the three aromatic amino acid residues.

To facilitate the therapeutic and non-therapeutic uses of the mTOR agonist/s and combinations disclosed herein, the present disclosure further provides compositions comprising the mTOR agonist/s and combinations of the invention.

In some particular and non-limiting embodiments, the mTOR agonist/s and any dosage forms thereof, as disclosed herein comprise all three aromatic amino acid residues Y, W, F, in an effective amount as disclosed herein above. More specifically, in some embodiments, the mTOR agonist/s of the invention may comprise the aromatic amino acids Y, W and F, in a concentration ranging between about 0.01 mM to about 30 mM or more, provided that the concentration of each of the aromatic amino acid residues is less than 45 mM, and in some further embodiments, the concentration is no more than 35 mM, as discussed in connection with other aspects of the present disclosure. In yet some further embodiment, the mTOR agonist/s disclosed herein may comprise an amount of between about 5 gr-7 gr, to about 50 gr-70 gr of each of the aromatic amino acid residues Y, W, F. In yet some further embodiments, the effective amount used in the mTOR agonist/s disclosed herein may range between about 0.1 gr per day/per kg to about 0.9 gr per day/per kg, for each of the aromatic amino acid residues Y, W, F, and in some embodiments, no more than 0.99 gr per day/per kg, for each of the aromatic amino acid residues Y, W, F. It should be appreciated however that the indicated effective doses per day, or dosage unit as discussed herein, may be given either in a single administration or in two or more administrations at several time-points over 24 hr. Still further, administration and doses are determined by good medical practice of the attending physician and may depend on the age, sex, weight and general condition of the subject in need.

Thus, a further aspect of the invention relates to a composition comprising as an active ingredient at least one mTOR agonist comprising at least one aromatic amino acid residue, any compound that modulates directly or indirectly at least one of the levels, stability and bioavailability of the at least one aromatic amino acid residue, any combinations or mixtures thereof, any vehicle, matrix, nano- or micro-particle thereof, optionally in a least one dosage form or at least one dosage unit form. In some specific embodiments, the composition of the invention may comprise any of the mTOR agonist/s of the invention, specifically, any of the mTOR agonist/s disclosed herein, or any vehicle, matrix, nano- or micro-particle thereof. In some embodiments, the composition may optionally further comprise at least one pharmaceutically acceptable carrier/s, excipient/s, auxiliaries, and/or diluent/s.

In yet some more specific embodiments, the mTOR agonist comprised within the composition provided by the present disclosure may comprise at least one aromatic amino acid residue or a combination of at least two aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In some specific embodiments, the mTOR agonist of the compositions disclosed herein may comprise at least two of the following components, optionally, in at least one dosage form or at least one dosage unit form. First component (a), comprises at least one tyrosine (Y) residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist may comprise in some embodiments as the second component (b), at least one tryptophan (W) residue, any mTOR agonistic tryptophan mimetic, any salt or ester thereof, any multimeric and/or polymeric form of the tryptophan residue and/or of the mTOR agonistic tryptophan mimetic, or any combination or mixture thereof. In yet some further embodiments, the mTOR agonist of the invention may comprise (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof.

In some embodiments, the mTOR agonist in accordance with the composition of the invention may comprise at least one tyrosine residue, any mimetic, any salt or ester thereof, any multimeric and/or polymeric form thereof, and any combinations or mixtures thereof, and at least one tryptophane residue, any mimetic, any salt or ester thereof, any multimeric and/or polymeric form thereof, and any combinations or mixtures thereof. In some further embodiments, the mTOR agonist in accordance with the composition of the invention may comprise at least one tyrosine residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. In yet some further embodiments, the mTOR agonist in accordance with the composition of the invention may comprise at least one tryptophane residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. Still further, in some specific embodiments, the mTOR agonist of the composition of the present disclosure may comprise a combination of the following three components, optionally, in at least one dosage form or at least one dosage unit form, or alternatively, in two or three dosage unit forms. More specifically, the composition may comprise: a first component (a), comprising at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof, optionally, in a dosage unit form. The mTOR agonist of the invention further comprises component (b), comprising at least one tryptophan residue, any mTOR agonistic tryptophan mimetic, any salt or ester thereof, any multimeric and/or polymeric form of the tryptophan residue and/or of the mTOR agonistic tryptophan mimetic, or any combination or mixture thereof, optionally, in a dosage unit form. The mTOR agonist of the composition of the present disclosure further comprises component (c), comprising at least one phenylalanine residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof, optionally, in at least one dosage form or at least one a dosage unit form.

The aromatic amino acid residues indicated above and throughout the present disclosure, as “first” or “first component”, “second” or “second component”, “third” or “third component”. However, it should be understood that the indication of first, second and third is used herein only for simplification purpose. Moreover, the composition of the invention may comprise only the first and second components, the first and third components, the second and third components, all three components, or any combinations thereof with any other component, or any one of the first, second or third components either alone or at any combination.

In some embodiments, in addition to the mTOR agonist/s, the compositions of the invention may further comprise an effective amount of at least one UPS modulating agent, specifically, at least one proteasome inhibitor and/or PROTAC and/or selective modulators, specifically inhibitors, of proteasome translocation, and/or additional therapeutic agent. It should be understood that any known UPS-modulating agent, for example, any known proteasome inhibitor and/or PROTAC may be used herein. In some specific embodiments, any of the UPS-modulators, for example, the proteasome inhibitors disclosed by the present disclosure in connection with other aspects, are also applicable for the compositions provided herein. Still further, in some embodiments, the composition may further comprise any selective modulator of proteasome translocation, specifically, a selective inhibitor of proteasome translocation. In some embodiments, the composition may further comprise at least one additional therapeutic agent, for example, at least one agent enhancing a stress condition or process, or specifically, in some embodiments, a short-term stress-condition or disorder, or alternatively, enhancing cytosolic proteasomal localization and/or activity. In more specific embodiments, the short-term stress condition or process may be any stress condition that induces or involved in nuclear-cytosolic proteasomal translocation. In more specific embodiments, the additional therapeutic agent may be at least one agent that leads to, enhances, and/or aggravates hypoxia, for example, agents that inhibit or reduce angiogenesis. Specific agents that inhibit angiogenesis applicable for the present disclosure are indicated herein below. Still further, any agent or procedure that results in starvation, e.g., a restricted diet, may be also used herein to further enhance stress.

In yet some further embodiments, the compositions of the invention may comprise in addition to, or instead of, the at least one aromatic amino acid residue or any mimetics thereof, any compound that modulates directly or indirectly at least one of the levels, stability and bioavailability of the at least one aromatic amino acid residue, optionally in at least one dosage unit form. Non-limiting examples for such compound include Nitisinone, that may increase the levels of tyrosine and/or phenylalanine.

Still further, it should be understood that any of the mTOR agonist/s of the invention, specifically any of the aromatic amino acid residues disclosed herein (either the D-isomers of YWF, the L-isomers of YWF or any mixtures thereof) or any mimetics thereof, or any peptide or protein comprising the at least one aromatic amino acid residues of the invention or any mimetics thereof, may be in certain embodiments, associated with, combined with or conjugated with at least one “enhancing” moiety. Such moiety may be any moiety that increases the mTOR agonistic effect thereof, and specifically, promotes and/or enhances proteasome nuclear localization, and/or activity, either by facilitating cell penetration, targeting to specific cell target and/or by increasing stability and reducing clearance thereof. The term “associated with” as used herein in reference to a half-life increasing moiety, a cell penetration moiety, a specific tissue or organ-directing moiety or a specific cell type directing moiety means that such moiety may be linked non-covalently, or covalently bound to, conjugated to, cross-linked to, incorporated within (e.g., such as an amino acid sequence within a peptide, polypeptide or protein that comprise at least one of the aromatic amino acid residues of the invention or any mimetics thereof), or present in the same composition as the at least one aromatic amino acid residue (specifically, Y, W and/or F), any mimetics thereof, a peptide comprising the at least one amino acid residues, non-standard peptide, polypeptide, non-standard polypeptide, protein or non-standard protein comprising the aromatic amino acid residues of the invention, in such a way as to allow such moiety to carry out its function. The term “cell penetration moiety” as used herein means a moiety that enhances the ability of the peptide, non-standard peptide, polypeptide, non-standard polypeptide, protein or nonstandard protein thereof with which it is associated to penetrate the cell membrane. In some embodiments, the “cell penetration moiety” may be an amino acid sequence within or connected to a peptide comprising at least one of the aromatic amino acid residues of the invention, non-standard peptide, polypeptide, non-standard polypeptide, protein or non-standard protein. Examples of cell penetration sequences include, but are not limited to, Arg-Gly-Asp (RGD), Tat peptide, oligoarginine, MPG peptides, Pep-1 and the like.

The term “specific organ directing moiety” as used herein means a moiety that enhances the ability of the aromatic amino acid residue/s of the invention or any mimetics thereof, peptide, non-standard peptide, polypeptide, non-standard polypeptide, protein or non-standard protein thereof, with which it is associated to be targeted to a specific organ. In some embodiments, the “specific organ directing moiety” is an amino acid sequence, small molecule or antibody that binds to a cell type present in the specific organ. In some embodiments, the “specific organ directing moiety” is an amino acid sequence, small molecule or antibody that binds to a receptor or other protein characteristically present in the specific organ.

The term “specific cell-type directing moiety” as used herein means a moiety that enhances the ability of the aromatic amino acid reside, or any peptide, non-standard peptide, polypeptide, non-standard polypeptide, protein or non-standard protein thereof, with which it is associated to be targeted to a specific cell type. In some embodiments, the “specific cell-type directing moiety” is an amino acid sequence, small molecule or antibody that binds to a specific receptor or other protein characteristically present in or on the surface of the specific target cell type.

The mTOR agonist/s of the present disclosure, specifically, at least one of tyrosine, tryptophan and/or phenylalanine (Y, W and/or F), and mimetics thereof, any dosage form or any dosage unit form thereof, may be formulated into a pharmaceutically acceptable composition or a nutraceutical composition. Such composition may, for example, be designed for any suitable administration mode, that may be adapted to any desired tissue, organ or cell. Non-limiting examples for administration modes include but are not limited to, parenteral, enteral, intra-muscular, direct to brain, or oral administration. Further relevant administration modes are discussed herein after. In a more specific aspect, at least one of the mTOR agonist/s or any dosage form or dosage unit form thereof, is formulated into a controlled release formulation. In this connection, the use of implant that acts to retain the active dose at the site of implantation, is also encompassed by the invention. The active agent may be formulated for immediate activity, or alternatively, or it may be formulated for sustained release as mentioned herein.

In another more specific aspect, at least one of the mTOR agonist/s is formulated into a composition to promote absorption from a specific portion of the target organ. In even more specific embodiments, any of the compositions of the present disclosure may be formulated as a pharmaceutical composition for delivery to a specific organ or cell type (e.g., brain, muscle, fibroblasts, bone, cartilage, liver, lung, breast, skin, bladder, kidney, heart, smooth muscle, adrenal, pituitary, pancreas, melanocytes, blood, adipose, and intestine). It will be understood that formulation for delivery to the brain requires the ability of the active components to cross the blood-brain barrier or to be directly administered to the brain or CNS.

As indicated above, the compositions of the invention may comprise in some embodiments, at least one additional therapeutic agent, specifically, agents enhancing a stress condition or process. In more specific embodiments, the stress condition or process may be any stress condition that induces or involved in proteasomal cellular shuttling and translocation, for example, nuclear-cytosolic or cytosolic-nuclear proteasomal translocation. In certain embodiments, such stress conditions or processes include at least one of hypoxia, amino acid starvation and/or unfolded protein response (UPR) stress. In more specific embodiments, the additional therapeutic agent may be at least one agent that leads to, enhances, and/or aggravates hypoxia. In some specific embodiments, agents that lead to or cause hypoxia, may be agents that inhibit or reduce angiogenesis. More specifically, angiogenesis as used herein, is a process involving the formation of new blood vessels. Angiogenesis is a characteristic phenomenon in numerous diseases, such as tumor formation, rheumatoid arthritis, diabetic retinopathy, and psoriasis to name but a few. This process involves the migration, growth, and differentiation of endothelial cells, which line the inside wall of blood vessels. The process of angiogenesis is controlled by various factors such as vascular endothelial growth factor (VEGF), angiopoietins (Ang), platelet-derived growth factor (PDGF), matrix metalloproteinase (MMP) which expedite cell proliferation, tube formation and migration of endothelial cells. These molecules serve as targets for angiogenesis inhibitors that block the growth of blood vessels and/or interfere with various steps in blood vessel growth. A wide variety of compounds has been reported to exhibit anti-angiogenic activity through various molecular pathways. Apart from antagonistic VEGF, for example by using antibodies that specifically recognize and bind VEGF, small molecules such as vatalanib, tivozanib, cediranib, and lenvatinib have been shown to inhibit receptor tyrosine kinase (RTK) signalling, thereby affecting angiogenesis. Plant polyphenols, catechins, flavonoids, terpenes, tannins, alkaloids and polyacetylenes comprise the natural anti-angiogenic phytochemicals. Compounds such as taxol, camptothecin and combretastatin have been reported to have potent anti-angiogenic properties.

Further, anti-angiogenic effects through inhibition of VEGF signalling have been reported from dietary functional foods such as genistein from soybean, epigallocatechin gallate from green tea, and resveratrol from red grapes.

As indicated above, the mTOR agonists of the present disclosure may be combined with, or administered at a combined therapeutic treatment regimen together with at least one angiogenesis inhibitor, that may be directed at VEGF (e.g., VEGF specific antibodies) or at any angiogenesis factor, for example, any of the factors discussed above. Non-limiting examples of angiogenesis inhibitors useful in the methods, compositions and kits of the present disclosure include at least one of: VEGF inhibitors, for example, anti-VEGF antibodies such as Bevacizumab (Avastin®), and Ramucirumab (Cyramza®), VEGF fusion proteins such as Ziv-aflibercept (Zaltrap®), kinase inhibitors such as Vandetanib (Caprelsa®), Sunitinib (Sutent®), Sorafenib (Nexavar®), Regorafenib (Stivarga®), Pazopanib (Votrient®), Cabozantinib (Cometriq®), Axitinib (Inlyta®), and agents involved with degradation of proteins (e.g., via interaction with E3 ligases) such as Thalidomide (Synovir, Thalomid®), and related drugs, for example, Lenalidomide (Revlimid®). More specifically, Axidnib (Inlyta®), a small molecule tyrosine kinase inhibitor, is used as a treatment option for kidney cancer. Bevacizumab (Avastin®), is a recombinant humanized monoclonal antibody that blocks angiogenesis by inhibiting VEGF-A. Avastin is used in the treatment of colorectal, kidney, and lung cancers. Cabozantinib (Cometriqg), is a small molecule inhibitor of the tyrosine kinases c-Met and VEGFR2, and also inhibits AXL and RET. Cabozantinib is used in the treatment of medullary thyroid cancer and kidney cancer. Lenalidomide (CC-5013; IMID3; Revlimid®), having the Formula C₁₃H₁₃N₃O₃, is an analogue of thalidomide, a glutamic acid derivative with anti-angiogenic properties and potent anti-inflammatory effects owing to its anti-tumor necrosis factor (TNF)α activity, and is therefore classified as an Imunomodulatory drug (IMiD). Lenalidomide is used as a treatment option for multiple myeloma and mantle cell lymphoma, which is a type of non-Hodgkin lymphoma. Lenvatinib mesylate (Lenvima®), having the formula C₂₁H₁₉ClN₄O₄, acts as a multiple kinase inhibitor against the VEGFR1, VEGFR2 and VEGFR3 kinases, and is used for the treatment of certain kinds of thyroid cancer. Pazopanib (Votrient®), having the formula C₂₁H₂₃N₇O₂S, is a potent multi-targeted receptor tyrosine kinase inhibitor, that inhibits VEGFR, PDGFR, c-KIT and FGFR. Pazopanib is used as a treatment option for kidney cancer and advanced soft tissue sarcoma. Ramudrumab (Cyramza®), is a fully human monoclonal antibody (IgG1) that binds with high affinity to the extracellular domain of VEGFR2 and block the binding of natural VEGFR ligands (VEGF-A, VEGF-C and VEGF-D). Ramucirumab is used in the treatment of advanced stomach cancer; gastroesophageal junction adenocarcinoma, colorectal cancers; and non-small cell lung (NSCL) cancers. Regorafenib (Stivarga), having the formula C₂₁H₁₅ClF₄N₄O₃, is an oral multi-kinase inhibitor that display dual inhibitory activity on VEGFR2-TIE2. Regorafenib is used as a treatment option for colorectal cancer and gastrointestinal stromal tumors (GIST). Sorafenib (Nexavar®), having the formula C₂₁H₁₆ClF₃N₄O₃, is a protein kinase inhibitor of various protein kinases, including VEGFR, PDGFR and RAF kinases. This drug is used in the treatment of kidney, liver, and thyroid cancers. Sunitinib (Sutent®), is an oral, small-molecule, multi-targeted receptor tyrosine kinase (RTK) inhibitor having the formula C₂₂H₂₇FN₄O₂, that blocks the tyrosine kinase activities of KIT, PDGFR, VEGFR2 and other tyrosine kinases. Sunitinib is used as a treatment option for kidney cancer, PNETs, and GIST. Thalidomide (Synovir, Thalomid®) (α-N-phthalimido-glutarimide), is a synthetic derivative of glutamic acid, which was know for causing birth defects when used as an antiemetic in pregnancy in the late 1950s and early 1960s. As indicated above, Thalidomide and its analogs are IMiDs. These drugs bind CRBN, a substrate receptor of CRL4 E3 ligase, to induce the ubiquitination and degradation of IKZF1 and IKZF3. Thalidomide is used in the treatment of multiple myeloma. Vandetanib (Caprelsa), having the formula C₂₂H₂₄BrFN₄O₂, acts as a kinase inhibitor of a number of cell receptors, mainly the VEGFR, the EGFR, and the RET-tyrosine kinase. This drug is used as a treatment option for medullary thyroid cancer. Ziv-aflibercept (Zaltrap), is a recombinant fusion protein consisting of VEGF-binding portions of the extracellular domains of human VEGF receptors 1 and 2, that are fused to the Fc portion of the human IgG1 immunoglobulin. This drug is used in the treatment of wet macular degeneration and metastatic colorectal cancer. It should be appreciated that any of the anti-angiogenic agents disclosed herein are applicable as an additional therapeutic agent for any of the aspects of the present disclosure.

In some embodiments, the at least one mTOR agonist of the composition disclosed herein may be formulated as an oral dosage form. In yet some further embodiments, the composition disclosed herein may be formulated in an oral dosage unit form. In yet some alternative embodiments, the at least one mTOR agonist may be formulated as an injectable dosage form. In yet some further embodiments, the composition disclosed herein may be formulated in an injectable dosage unit form.

In some embodiments, the oral dosage form may be administered orally, for example, as a solution (e.g., syrup), or as a powder, tablet, capsule, and the like. In some further embodiments, the oral dosage form may be provided in a formulation adapted for add-on to a solid, semi-solid or liquid food, beverage, food additive, food supplement, medical food, drug and/or a pharmaceutical composition.

In certain embodiments the composition of the invention may be formulated in a formulation adapted for add-on to a solid, semi-solid or liquid food, beverage, food additive, food supplement, medical food, botanical drug, drug and/or any type of pharmaceutical compound.

In some embodiments, the add-on composition according to the invention may be formulated as a food additive, food supplement or medical food. In other embodiment, such add-on composition of the invention may be further added or combined with drugs or any type of pharmaceutical products. The term ‘add-on’ as used herein is meant a composition or dosage unit form of the at least one mTOR agonists of the present disclosure that may be added to existing compound, composition or material (e.g., food or beverage), enhancing desired properties thereof or alternatively, adding specific desired property to an existing compound, composition, food or beverage.

More specifically, in certain embodiments, the at least one mTOR agonists of the present disclosure, or any dosage form or composition thereof may be an add-on to a food supplement, or alternatively, may be used as a food supplement. A food supplement, the term coined by the European Commission for Food and Feed Safety, or a dietary supplement, an analogous term adopted by the FDA, relates to any kind of substances, natural or synthetic, with a nutritional or physiological effect whose purpose is to supplement normal or restricted diet. In this sense, this term also encompasses food additives and dietary ingredients. Further, under the Dietary Supplement Health and Education Act of 1994 (DSHEA), a statute of US Federal legislation, the term dietary supplement is defined as a product intended to supplement the diet that bears or contains one or more of the following dietary ingredients: a vitamin, a mineral, an herb or other botanical, a dietary substance for use by a subject to supplement the diet by increasing the total dietary intake, or a concentrate, metabolite, constituent, extract, or combination of any of the aforementioned ingredients Food or dietary supplements are marketed a form of pills, capsules, powders, drinks, and energy bars and other dose forms. Unlike drugs, however, they are mainly unregulated, i.e., marketed without proof of effectiveness or safety. Therefore, the European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet. However, the add-on dosage form or composition that comprise the at least one mTOR agonists provided herein, may be added to a meal or beverage consumed by the subject.

In yet some further embodiments, the mTOR agonist or any composition thereof, in accordance with the present disclosure may be an add-on to medical foods or may be consumed as a medical food. Further in this connection should be mentioned medical foods, which are foods that are specially formulated and intended for the dietary management of a disease that has distinctive nutritional needs that cannot be met by normal diet alone.

A medical food, as defined in section 5(b)(3) of the Orphan Drug Act (21 U.S.C. 360ee(b)(3)), is “a food which is formulated to be consumed or administered enterally under the supervision of a physician and which is intended for the specific dietary management of a disease or condition for which distinctive nutritional requirements, based on recognized scientific principles, are established by medical evaluation.” FDA considers the statutory definition of medical foods to narrowly constrain the types of products that fit within this category of food (21 CFR 101.9(j)(8)). Medical foods are distinguished from the broader category of foods for special dietary use by the requirement that medical foods be intended to meet distinctive nutritional requirements of a disease or condition, used under medical supervision, and intended for the specific dietary management of a disease or condition. Medical foods are not those simply recommended by a physician as part of an overall diet to manage the symptoms or reduce the risk of a disease or condition. Not all foods fed to patients with a disease, including diseases that require dietary management, are medical foods. Instead, medical foods are foods that are specially formulated and processed (as opposed to a naturally occurring foodstuff used in a natural state) for a patient who requires use of the product as a major component of a disease or condition's specific dietary management.

It is a specially formulated and processed product (as opposed to a naturally occurring foodstuff used in its natural state) for the partial or exclusive feeding of a patient by means of oral intake or other feeding means (e.g., a tube or catheter).

Also pertinent to the present context are any type of drugs or therapeutic compounds, that may be available as (but not limited to) a solution (e.g., tea), powder, tablet, capsule, elixir, topical, or injection. Thus, in further embodiments, the at least one mTOR agonist, any dosage form, dosage unit form, or composition thereof, may be an add-on to any type of drugs or therapeutic compounds administered orally, intravenously, intradermaly, by inhalation or intrarectaly.

In some embodiments, the at least one mTOR agonist, any dosage form, dosage unit form, or composition thereof may be adapted for add-on a food and/or beverage.

In this context, a beverage is any beverage including for example fruit or fruit-flavored drinks, flavored water or sodas, energy drinks, coffees, teas, milk, chocolate milk and nonalcoholic wines and beers. Food, as used herein is any dry, semi-dry, or liquid edible substance providing nutrients and or calories to the consuming subject. Food may be composed of natural or synthetic ingredients and any combinations thereof, and may provide carbohydrates, fat, fibers, vitamins and other nutrients. Exemplary food products can be, but are not limited to bakery products, such as bread, biscuits, cookies, cakes, pastries and the like; confectionery products such as chocolate or vegetarian or vegan chocolate, candy, gummy; dates products; dairy or dairy like (vegetarian) products such as yoghurt, cheeses, ice creams; formula such as infant formula; garnishes such as mayonnaise, ketchup and the like; frozen foods; protein and energy bars; savory snacks; and the like. It should be however understood that the mTOR agonist, as well as any compositions and formulations thereof disclosed by the invention, that may be comprised within any food or food additives as discussed herein above, may encompass any food or food additives, provided that the YWF, and specifically, any dosage forms thereof, are not naturally occurring, or cannot be considered as naturally occurring in such food or food additives. Specifically, in some embodiments, the YWF or any compositions thereof were added to such foods or food additives by the present invention. As such, in some embodiments, the YWF of the present disclosure and any dosage form, dosage unit form, and/or composition thereof, is not considered as a natural product.

As indicated above, in connection with the mTOR agonist of the present disclosure, each of the aromatic amino acid residues may be provided in a dosage form or in a dosage unit form. Dosage forms, as used herein, are pharmaceutical drug products in the form in which they are marketed for use, with a specific mixture of active ingredients (e.g., the YWF, and/or any mimetics thereof) and optionally, inactive components (excipients), in a particular configuration (such as a capsule shell, for example), and apportioned into a particular dose. In some embodiments, the term dosage form can also refer in some embodiments only to the pharmaceutical formulation of a drug product's constituent drug substance(s) and any blends involved.

As used interchangeably herein, “dosage units”, “dosage forms”, “oral or injectable dosage units”, “dosage unitforms”, “oral or injectable dosage unitforms” and the like refer to both, solid dosage forms as known in the art, or to a liquid dosage form. The dosage forms are intended for peroral use, i.e., to be swallowed (ingested), or even injected or applicated in any other means, either by a subject in need thereof, or for administration by a medical practitioner. The terms “active substance” or “active ingredient”, used herein interchangeably, refer to a therapeutically or physiologically active substance, specifically, the mTOR agonists disclosed herein, that provides a therapeutic/physiological effect to a patient, and can also refer to a mixture of at least two thereof.

In some embodiments, any of the mTOR agonists of the present disclosure, as well as any formulations, dosage forms, dosage unit forms, compositions, kits methods and uses thereof may be adapted for, or may involve at least one systemic and/or at least one non-systemic administration. The term “non-systemically” as herein defined refers to a localized route of administration, namely a route of administration which is not via the digestive tract and not parenterally. In embodiments of the disclosure, the non-systemic administration may be any administration mode, for example, intrathecal, intra-nasal, intra-ocular, intraneural, intra-cerebral, intra-ventricular, intra-cerebroventricular, intra-cranial, and subdural administration. In yet some further embodiments, the of the disclosure, the systemic administration may be any administration mode, for example, oral, intravenous, intramuscular, subcutaneous, topical, enteral (e.g., gastrointestinal tract, specifically, oral, rectal, sublingual, sublabial or buccal, by any one of injection, enema, catheter, applicator, or any oral or topical formulation), or parenteral.

In yet some further embodiments, the mTOR agonists of the present disclosure, as well as any formulations, dosage forms, dosage unit forms, compositions, kits methods and uses thereof may be formulated as injectable formulations, that may be used either for systemic or for non-systemic, or local administration. In further embodiments of the disclosure the said injectable formulation, specifically, aqueous or liquid formulation, is designed for administration to said subject by bolus administration. In other embodiments of the disclosure the said aqueous injectable formulation is designed for administration to said subject by infusion of no less than one minute and no more than 24 hours.

Thus, the present disclosure further provides an injectable aqueous formulation for non-systemic administration to a subject in need thereof, said formulation comprising as active ingredient the at least one mTOR agonists of the present disclosure or any combinations or formulations thereof, that may comprise in some embodiments, the concentration of from about 0.1 mM of each of the Y, W, F of the present disclosure or any mimetics thereof, to about 30 mM or each of said aromatic amino acids Y, W, F, or any mimetics thereof. In yet some further embodiments, the concentration is no more than 35 mM for each of the aromatic amino acid residues.

In the disclosed methods of treatment, the injectable formulation as herein defined is administered once, twice or more a day, every other day, a week, every two weeks, every three weeks, once, twice or more every four weeks, once every 5, 6, 7 or 8 weeks, once a month, once every two months, once every three months, once every four months, once every five months or once every six months, or even once twice or more a year.

In the disclosed mTOR agonists of the present disclosure, as well as any formulations, dosage forms, dosage unit forms, compositions, kits, uses and methods of treatment, the rate of administration of the injectable formulation disclosed herein is such that the maximum level of each of the aromatic amino acid residues, Y, W and F, is no more than 0.99 gr per kilogram of body weight of said subject per day, and in some embodiments, less than 1 gr per kg per day. In specific embodiments the said administration is performed by infusion of no less than one minute and no more than 24 hours.

As indicated herein, the composition or any dosage form or dosage unit form disclosed herein may be provided in an injectable formulation. The term “injecdon” or “injectable” as used herein refers to a bolus injection (administration of a discrete amount of the at least one mTOR agonists disclosed herein, for raising its concentration in a bodily fluid), slow bolus injection over several minutes, or prolonged infusion, or several consecutive injections/infusions that are given at spaced apart intervals. Such spaced apart injections per a single administration are also referred to herein as “per administradon injecdon”, or in other words, a single administration can include several injections or prolonged infusion. The injectable aqueous formulation for non-systemic administration to a subject in need thereof as herein defined may be administered using a drug-device combination, for example a mechanical or electro-mechanical device, more preferably an electro-mechanical infusion pump. The electro-mechanical pump, for example, consists of a reservoir for housing a medication, a catheter having a proximal portion coupled to the pump and having a distal portion adapted for administering a medication to the desired site.

Still further, the composition of the present disclosure, as well as any product or use of the mTOR agonist of the present disclosure, specifically, the YWF disclosed herein may be provided and/or used in an effective amount. More specifically, the compositions of the invention may comprise an effective amount of at least one mTOR agonist of the invention as disclosed herein and/or any vehicle, matrix, nano- or micro-particle thereof. The term “effective amount” relates to the amount of an active agent present in a composition, specifically, the mTOR agonist of the invention as described herein that is needed to provide a desired level of active agent in the bloodstream or at the site of action in an individual (e.g., the specific site of the tumor) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of the mTOR agonist/s of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

An effective amount in accordance with the mTOR agonists of the present disclosure, specifically mTOR agonist comprising the at least two aromatic amino acid residues, and more specifically, all three amino acid residues Tyrosine, Tryptophane and Phenylalanine, as used in the present disclosure (e.g., in the mTOR agonists, compositions, kits and methods disclosed herein), may be presented in any amount effective for selective and specific agonistic activity for mTOR mediated activities, specifically, in modulating proteasome dynamics in a cell, as discussed herein. In yet some further embodiments, the amount of the aromatic amino acid residues is any amount effective for specific and selective inhibition of proteasome recruitment or translocation from the nucleus to the cytosol. Still further, in some embodiments, an effective amount is an amount effective for specifically and selectively maintaining nuclear localization of the proteasome in cells of a subject in need. In yet some further embodiments, an effective amount is an amount effective for specifically and selectively requiring the proteasome into the nucleus and modulating proteasome dynamics such that the proteasome localization is predominantly nuclear in cells of the treated subject.

Thus, in some embodiments, the compositions of the invention comprise least one tyrosine (Y) residue, at least one tryptophan (W) residue, and at least one phenylalanine (F) residue, or any mTOR agonistic mimetic, salt or ester thereof, any multimeric and/or polymeric form thereof, and any combinations or mixtures thereof, and any dosage forms or dosage unit form thereof, in an amount effective for selective modulation of proteasome localization, specifically, selective and specific inhibition of proteasome translocation, specifically, inhibition of proteasome translocation to the cytosol, and optionally, selective and specific enhancement of recruitment of the proteasome to the nucleus, in at least one cell of at least one subject treated by the mTOR agonists, dosage forms, dosage unit forms, compositions, kits and methods disclosed herein.

As shown in the present disclosure, the three aromatic amino acid residues of the invention, specifically, tyrosine, trytophan, and phenylalanine (YWF), effectively and selectively, inhibit proteasome translocation to the cytosol in cells, and moreover, in some embodiments maintains and recruit proteasome to the nucleus. This has been demonstrated by the present disclosure in vitro and in vivo, when the aromatic amino acids of the invention were administered locally to the tumor, or systemically. Most importantly, when provided systemically, either by injectable or oral compositions, the triad, YWF, synergistically inhibited tumor cell growth, as well as tumor mass and tumor volume (FIGS. 14, 15, and 17-19). These synergistically effective amounts of all three aromatic amino acid residues, Y, W, F, have been converted and adapted herein for use in a mammalian subject, specifically, a human subject. As shown herein, specifically in Example 16, a concentration of about 0.01 to 30 mM for each of the aromatic amino acid resides YWF, is used. Specifically, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1 mM or more, specifically, 1.1, 1.2, 1.3, 1.4, 1.5, 1.6, 1.7, 1.8, 1.9., 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 15, 20, 25 or 30 mM or more. In some embodiments, the concentration of each of the aromatic amino acid residues, Y, W, F, or mimetics thereof in a dosage form may range between about 0.01 mM to about 30 mM or more, provided that the concentration of each of the aromatic amino acid residues is less than 45 mM. In yet some further embodiments, the concentration of each of the amino acid residues in the dosage form or composition disclosed herein is no more than 35 mM. In yet some further embodiments, the total concentration of all three aromatic amino acid residues of the invention is less than 45 mM. Still further, in some specific and non-limiting embodiments, the concentration of each one of the Y, W and F residues in the selective inhibitors of proteasome translocation and/or mTOR agonists, or any compositions, kits, dosage forms, and methods thereof may be 1.6 mM each. In yet some further embodiment, the concentration may be 6 mM for each. Thus, in some specific and non-limiting embodiments, each of the aromatic amino acid residues tyrosine, tryptophan, and phenylalanine (YWF) may be presented in an amount ranging between about 1 mg to about 100 gr or more in the composition of the invention or in any dosage form or dosage unit form disclosed herein, specifically, between about 0.001 gr to about 100 gr, more specifically, between about 0.01 gr to about 100 gr, between about 0.1 gr to about 100 gr, between about 1 gr to about 100 gr, between about 2 gr to about 100 gr, 3 gr to about 100 gr, 4 gr to about 100 gr, 5 gr to about 100 gr, 6 gr to about 100 gr, 7 gr to about 100 gr, 8 gr to about 100 gr, 9 gr to about 100 gr, 10 gr to about 100 gr, specifically, between about 10 gr to about 95 gr, 10 gr to about 90 gr, 10 gr to about 85 gr, 10 gr to about 80 gr, 10 gr to about 750 gr, 10 gr to about 65 gr, 10 gr to about 60 gr, 10 gr to about 55 gr, 10 gr to about 45 gr, 10 gr to about 40 gr, 10 gr to about 35 gr, 10 gr to about 30 gr, 10 gr to about 25 gr, 10 gr to about 20 gr, 10 gr to about 15 gr. In some specific embodiments, each of the aromatic amino acid residues tyrosine, tryptophan, and phenylalanine (YWF) may be present in an amount ranging between about 10 gr to about 20 gr in the composition of the invention. Still further, a dosage form, a dosage unit form or any compositions and kits disclosed herein may comprise an amount of between about 5 gr-7 gr, to about 50 gr-70 gr of each of the aromatic amino acid residues Y, W, F, (as calculated for an adult weighing between about 50 to 70 kg). In yet some further embodiments, an effective amount provided to a subject may range between about 0.01 gr to about 10 gr per day/per kg of body weight. In yet some further embodiments, the effective amount used in the dosage forms, formulations, compositions, kits and methods disclosed herein may range between about 0.1 gr per day/per kg to about 0.9 gr per day/per kg, for each of the aromatic amino acid residues Y, W, F. In yet some further embodiments, the dosage forms, formulations, compositions, kits and methods disclosed herein is no more than 0.99 gr per day/per kg, for each of the aromatic amino acid residues Y,W, F. In more specific embodiments, about 0.01 gr, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.5, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 gr/kg/day or more, to about 1 gr per day/per kg or less. In more specific embodiments, each of the aromatic amino acid residues tyrosine, tryptophan, and phenylalanine (YWF) may be present in an amount ranging between about 0.1 to about 0.9 gr/day/kg. More specifically, about 0.1 gr per day/per kg, for each of the aromatic amino acid residues Y,W,F, or between about 0.1 to about 0.2 gr per day/per kg, about 0.2 gr per day/per kg or between about 0.2 to about 0.3 gr per day/per kg, about 0.3 gr per day/per kg or between about 0.3 to about 0.4 gr per day/per kg, about 0.4 gr per day/per kg or between about 0.4 to about 0.5 gr per day/per kg, about 0.5 gr per day/per kg or between about 0.5 to about 0.6 gr per day/per kg, about 0.6 gr per day/per kg or between about 0.6 to about 0.7 gr per day/per kg, about 0.7 gr per day/per kg or between about 0.7 to about 0.8 gr per day/per kg, about 0.8 gr per day/per kg or between about 0.8 to about 0.9 gr per day/per kg, about 0.9 gr per day/per kg or between about 0.9 to about but no more than 0.99 gr per day/per kg, and in some embodiments, less than 1 gr per day/per kg, for each of the aromatic amino acid residues Y,W,F. It should be appreciated however that the indicated effective doses per day, or dosage unit as discussed herein, may be given either in a single administration or in two or more administrations at several time-points over 24 hr. Still further, administration and doses are determined by good medical practice of the attending physician and may depend on the age, sex, weight and general condition of the subject in need.

It should be appreciated that the effective amount as discussed herein is applicable for each and every embodiment of each and every aspect of the present disclosure, specifically, for any of the mTOR agonists, any dosage forms thereof, dosage unit forms thereof, compositions, kits, uses and methods thereof.

The pharmaceutical compositions of the invention can be administered and dosed by the methods of the invention, in accordance with good medical practice, systemically, for example by parenteral, e.g., intrathymic, into the bone marrow, peritoneal or intraperitoneal, specifically administered to any peritoneal cavity, and any direct administration to any cavity or organ, specifically, the pleural cavity (mesothelioma, invading lung) the urinary bladder and to the brain.

It should be noted however that the invention may further encompass any additional administration modes. In other examples, the pharmaceutical composition can be introduced to a site by any suitable route including subcutaneous, transcutaneous, topical, intramuscular, intraarticular, subconjunctival, or mucosal, intravenous, e.g., oral, intranasal, intraocular administration, or intra-tumor as well.

Still further, local administration to the area in need of treatment may be achieved by, for example, by local infusion during surgery, or using any permanent or temporary infusion device, topical application, direct injection into the specific organ, etc. More specifically, the compositions disclosed herein, that are also used in any of the methods of the invention, described in connection with other aspects of the present disclosure, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). In some optional embodiment, the agonists of the present invention as well as any formulations thereof may be administered directly to the central nervous system (CNS). Examples of direct administration into the CNS include intrathecal administration, and direct administration into the brain, such as intra-cerebral, intra-ventricular, intra-cerebroventricular, intra-cranial or subdural routes of administration. Such routes of administration may be particularly beneficial for diseases involving or requiring cytosolic proteasome accumulation and/pr increased activity of the proteasome in the cytosol, that may in some embodiments affect the central nervous system (e.g., benign or malignant tumors of any neuronal or brain tissue).

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the mTOR agonist of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations, or formulations comprising any other nan- or micro-particles or any matrix comprising the at least one mTOR agonist disclosed herein.

It should be understood that in addition to the ingredients particularly mentioned above, the formulations may also include other agents conventional in the art having regard to the type of formulation in question.

As indicated above, pharmaceutical preparations are compositions that include one or more mTOR agonist present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in any organism, specifically any vertebrate organism, for example, any mammal such as human. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the mTOR agonist/s of the invention can be achieved in any of the various ways disclosed by the invention.

As noted above, the present invention involves the use of different active ingredients, specifically, the mTOR agonists of the present disclosure, for example, the tyrosine, tryptophan and phenylalanine, and optionally, at least one UPS-modulating agent, for example, at least one proteasome inhibitor, and/or any additional therapeutic compound that may enhance stress condition or process, that may be administered through different routes, dosages and combinations. More specifically, the treatment of disorders associated with at least one short term stress condition, as well as any conditions associated therewith, with a combination of active ingredients may involve separate administration of each active ingredient. Therefore, a kit providing a convenient modular format for the combined therapy using the mTOR agonists of the invention, specifically, the at least one aromatic amino acid residues, tyrosine, tryptophan and phenylalanine, required for treatment, would allow the desired or preferred flexibility in the above parameters. Thus, a further aspect of the invention relates to a kit comprising at least two, or a combination of at least two of:

First (a), at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof, optionally, in a first dosage form. In some embodiments, the kits of the invention may comprise additionally, or alternatively, (b), at least one tryptophan residue, any mTOR agonistic tryptophan mimetic, any salt or ester thereof, any multimeric and/or polymeric form of the tryptophan residue and/or of the mTOR agonistic tryptophan mimetic, or any combination or mixture thereof, optionally, in a second dosage form. In yet some further embodiments, the kit of the invention may comprise additionally, or alternatively (c), at least one phenylalanine residue, any mTOR agonistic phenylalanine mimetic, any salt or ester thereof, any multimeric and/or polymeric form of the phenylalanine residue and/or of said mTOR agonistic phenylalanine mimetic, and any combinations or mixtures thereof, optionally, in a third dosage form.

In some embodiments, the mTOR agonist in accordance with the kits of the invention may comprise at least one tyrosine residue, any mimetic, any salt or ester thereof, any multimeric and/or polymeric form thereof, and any combinations or mixtures thereof, and at least one tryptophane residue, any mimetic, any salt or ester thereof, any multimeric and/or polymeric form thereof, and any combinations or mixtures thereof. In some further embodiments, the mTOR agonist in accordance with the kits of the invention may comprise at least one tyrosine residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. In yet some further embodiments, the mTOR agonist in accordance with the kits of the invention may comprise at least one tryptophane residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. Still further, in some specific embodiments, the mTOR agonist provided by the kit of the present disclosure may comprise all three aromatic amino acid residue as discussed above, or a combination of the three aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In more specific embodiments, the mTOR agonist of the kit/s of the present disclosure may comprise a combination of the following three components: first component (a), comprises at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist of the invention further comprises component (b), at least one tryptophan residue, any mTOR agonistic tryptophan mimetic, any salt or ester thereof, any multimeric and/or polymeric form of the tryptophan residue and/or of the mTOR agonistic tryptophan mimetic, or any combination or mixture thereof. The mTOR agonist of the kit of the present disclosure further comprises component (c), at least one phenylalanine residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof.

Still further, in some embodiments, the kits of the invention may comprise in addition to, or instead of, the at least one aromatic amino acid residue or any mimetics thereof, any compound that modulates directly or indirectly at least one of the levels, stability and bioavailability of the at least one aromatic amino acid residue. Non-limiting examples for such compound include Nitisinone, that may increase the levels of tyrosine and/or phenylalanine. In yet some additional specific embodiments, the kits of the invention may comprise either alternatively or additionally, at least one moiety that increases the mTOR agonistic effect of the mTOR agonist/s of the invention, and specifically, promotes and/or enhances proteasome nuclear localization, either by facilitating cell penetration, targeting to specific cell target or increasing stability and reducing clearance thereof. In some embodiments, the kit of the invention may further comprise at least one UPS-modulating agent, for example, at least one proteasome inhibitor, or any of the modulators disclosed by the invention, optionally, in a fourth dosage form.

In some embodiments, the kit may further comprise at least one additional therapeutic agent, for example, at least one agent enhancing a short-term stress condition or process. For example, agents that leads to, enhances, and/or aggravates hypoxia. In some specific embodiments, such agents may inhibit or reduce angiogenesis. Non-limiting examples of angiogenesis inhibitors useful in the methods, compositions and kits of the present disclosure include at least one of: VEGF inhibitors, for example, anti-VEGF antibodies or VEGF fusion proteins, kinase inhibitors and agents involved with degradation of proteins. In some embodiments, at least one of the at least two aromatic amino acid residues of the kit disclosed herein may be formulated in a dosage unit form. In some embodiments, the at least one mTOR agonist of the kits disclosed herein may be formulated as an oral dosage form. In yet some alternative embodiments, the at least one mTOR agonist may be formulated as an injectable dosage form.

In some embodiments, the oral dosage forms provided by the kits of the invention may be administered orally, for example, as a solution (e.g., syrup), as a powder, tablet, capsule, and the like. In some further embodiments, the oral dosage form may be provided in a formulation adapted for add-on to a solid, semi-solid or liquid food, beverage, food additive, food supplement, medical food, drug and/or a pharmaceutical composition.

In some particular and non-limiting embodiments, the kits disclosed herein comprise all three aromatic amino acid residues Y, W, F, in an effective amount as disclosed herein above. More specifically, in some embodiments, the kits of the invention may comprise the aromatic amino acids Y, W and F, in a concentration ranging between about 0.01 mM to about 30 mM or more, provided that the concentration of each of the aromatic amino acid residues is less than 45 mM, and in some further embodiments, the concentration is no more than 35 mM, as discussed in connection with other aspects of the present disclosure. In yet some further embodiment, the kits disclosed herein may comprise an amount of between about 5 gr-7 gr, to about 50 gr-70 gr of each of the aromatic amino acid residues Y, W, F. In yet some further embodiments, the effective amount used in the kits disclosed herein may range between about 0.1 gr per day/per kg to about 0.9 gr per day/per kg, for each of the aromatic amino acid residues Y, W, F, and in some embodiments, no more than 0.99 gr per day/per kg, for each of the aromatic amino acid residues Y,W, F.

It should be appreciated that any of the kit/s disclosed by the present disclosure may be applicable and used for any of the methods disclosed by the present invention.

As shown by the following Examples, the mTOR agonist/s of the invention remarkably modulate proteasome dynamics in cells of a treated subject, and therefore, display a clear clinical application. Therefore, a further aspect of the invention relates to a method for treating, preventing, inhibiting, reducing, eliminating, protecting or delaying the onset of at least one condition or at least one pathologic disorder involved, or associated with cytosolic proteasomal localization and/or activity in a subject. More specifically, the methods may comprise the step of administering to the subject an effective amount, or in some embodiments a therapeutically effective amount of at least one mTOR agonist comprising at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compound that modulates directly or indirectly at least one of the levels, stability and bioavailability of the at least one aromatic amino acid residue, any combinations or mixtures thereof, any vehicle, matrix, nano- or micro-particle thereof, or any dosage form, dosage unit forms, composition or kit comprising the same.

The present disclosure provides therapeutic and prophylactic methods applicable for any condition or pathologic disorder that requires, is associated with, or is characterized by, cytosolic localization, accumulation and/or activity of the proteasome. More specifically, the methods discussed herein are applicable for any disorder or condition characterized with, or defined by, predominant proteasome cytosolic localization, or by accumulation of the proteasome in the cytosol and/or increased activity of the proteasome in the cytosol, specifically, as compared with cells of a healthy subject or of a subject not suffering from the indicated disorder. In some embodiments, the disorders discussed herein may be any disorders characterized with proteasome malfunction, that may refer in some embodiments to increased activity. As indicated herein, the increased amount and/or activity of the proteasome in the cytosol of cells of the subject, is essential for providing the unmet need, or demand of the cells for energy sources, amino acids and/or recycled building blocks required for cell survival, and activity. Still further, the proteasome activity, as referred to herein, refers to proteolytic degradation of various cytoplasmic and nuclear proteins. The proteasome activity can be measured by any known methods. that may include for example, the use of fluorescently tagged proteasome subunits and the use of activity-based proteasome probes. Methods for determining proteasome localization are discussed herein after in connection with other aspects of the invention. Increased proteasomal degradation was measured in muscle wasting diseases, ischemic disorders, or any disorder or condition involving any catabolic process, hypercatabolic and/or hypermetabolic conditions. More specifically, as used herein, increased amount or activity of the proteasome in the cytosol of cells of the subject that suffers from the indicated disorder, means an increase or enhancement of at least about 10% or more, as compared to a reference level of the proteasome cytosolic localization and/or activity in cells of a subject that is not suffering from the indicated disorder. For example an increase of at least about 20%, or at least about 30%, or at least about 40%, or at least about 50%, or at least about 60%, or at least about 70%, or at least about 80%, or at least about 90% or up to and including a 100% increase, or at least about a 2-fold, or at least about a 3-fold, or at least about a 4-fold, or at least about a 5-fold or at least about a 10-fold increase, or any increase between 2-fold and 10-fold or greater as compared to a reference level (e.g., in subject not suffering from the disclosed disorders), of the proteasome cytosolic localization and/or activity.

Still further, in some embodiments, the therapeutic methods of the present disclosure may be applicable in some embodiments to conditions associated with cellular stress. In some particular embodiments, such stress may be any short or long-term stress, or at least one short or long term cellular condition or process that may be any stress condition inducing nuclear-cytosolic proteasomal translocation or in some non-limiting embodiments, an increased cytosolic localization of the proteasome. Still further, in some embodiments such disorders may be condition or process in which adequate cytosolic localization and/or activity of the proteasome is required for cell survival. Thus, in some embodiments, such disorders may display an average or normal proteasome amount in the cytosol, but however are characterized in dependency and requirement for cytosolic proteasome for cell survival. In some embodiments, such stress conditions include at least one of amino acid starvation, hypoxia and unfolded protein response (UPR) mediated proteasomal translocation.

More specifically, Amino acid starvation as used herein, relates but is not limited to nutrient deprivation, specifically amino acids and is marked by several distinctive physiological markers, including the induction of eIF2a phosphorylation, and the increased transcription of many stress responses. Amino acid starvation response (AAS), a broad-based cellular response, may be triggered or induced by starvation for many of the 20 amino acids, including but not limited to proline and essential amino acids such as phenylalanine, tryptophan, valine, threonine, isoleucine, methionine, leucine, lysine, histidine, etc. The amino acid response pathway is triggered by shortage of any essential amino acid, and results in an increase in activating transcription factor ATF4, which in turn affects many processes by sundry pathways to limit or increase the production of other proteins. At low concentration of amino acid, GCN2 is activated due to the increase level of unchanged tRNA molecules. Activated GCN2 phosphorylates itself and elF2α, it triggers a transcriptional and translational response to restore amino acid homeostasis by affecting the utilization, acquisition, and mobilization of amino acid in an organism. Essential amino acids are crucial to maintain homeostasis within an organism. It should be however understood that amino acid starvation as used herein further encompasses in addition to conditions characterized with depletion or depravation of amino acids, but also conditions in which increasing demand of the cells for energy sources, amino acids and/or recycled building blocks required for cell survival is unmet. Pathologic conditions associated with such starvation (that results from either depletion or increased unmet need) include, but are not limited to proliferative disorders, such as cancer, ischemic conditions, and any conditions associated with hypermetabolic and/or hypercatabolic conditions (e.g., muscle wasting diseases or conditions).

In yet some further embodiments, the stress condition, or in some embodiments, short-term stress condition relevant to the methods of the present disclosure may be hypoxia. Hypoxia is a condition in which the body or a region of the body is deprived of adequate oxygen supply at the tissue level. Hypoxia may be classified as either generalized, affecting the whole body, or local, affecting a region of the body. Although hypoxia is often a pathological condition, variations in arterial oxygen concentrations can be part of the normal physiology, for example, during hypoventilation training or strenuous physical exercise. Hypoxia differs from hypoxemia and anoxemia in that hypoxia refers to a state in which oxygen supply is insufficient, whereas hypoxemia and anoxemia refer specifically to states that have low or zero arterial oxygen supply. Hypoxia in which there is complete deprivation of oxygen supply is referred to as anoxia. Pathologic conditions associated with hypoxia include, but are not limited to proliferative disorders, such as cancer, or ischemic conditions.

Still further, a short-term stress condition applicable in the present disclosure may be UPR. More specifically, the Unfolded Protein Response (UPR) is a cellular stress response related to the endoplasmic reticulum (ER) stress. It has been found to be conserved between all mammalian species, as well as yeast and worm organisms. The UPR is activated in response to an accumulation of unfolded or misfolded proteins in the lumen of the endoplasmic reticulum. In this scenario, the UPR has three aims: initially to restore normal function of the cell by halting protein translation, degrading misfolded proteins, and activating the signaling pathways that lead to increasing the production of molecular chaperones involved in protein folding. If these objectives are not achieved within a certain time span or the disruption is prolonged, the UPR aims towards apoptosis.

It is interesting to note that sustained over-activation of the UPR has been implicated in prion diseases as well as several other neurodegenerative diseases and inhibiting the UPR could become a treatment for those diseases. Diseases amenable to UPR inhibition include Creutzfeldt-Jakob disease, Alzheimer's disease, Parkinson's disease, and Huntington's disease.

In some specific embodiments, the at least one mTOR agonist used by the methods of the invention may comprise at least one of: (a), at least one tyrosine residue, any mTOR agonistic tyrosine mimetic, any salt or ester thereof, any multimeric and/or polymeric form of the tyrosine residue and/or of said mTOR agonistic tyrosine mimetic, and any combinations or mixtures thereof, optionally, in a first dosage form. In some embodiments, the mTOR agonist used by the methods of the invention may comprise additionally or alternatively, (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof, optionally, in a second dosage form. In yet some further embodiments, the mTOR agonist/s used by the methods of the present disclosure may comprise additionally or alternatively, (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof, optionally, in a third dosage form. In some embodiments, the mTOR agonist used by the methods of the invention may comprise at least one tyrosine residue, any mimetic, any salt or ester thereof, any multimeric and/or polymeric form thereof, and any combinations or mixtures thereof, and at least one tryptophane residue, any mimetic, any salt or ester thereof, any multimeric and/or polymeric form thereof, and any combinations or mixtures thereof. In some further embodiments, the mTOR agonist used by the methods of the invention may comprise at least one tyrosine residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. In yet some further embodiments, the mTOR agonist used by the methods of the invention may comprise at least one tryptophane residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. In some embodiments, the mTOR agonist used by the methods of the invention comprise all three aromatic amin acid residues, specifically, (a), at least one tyrosine residue, any mTOR agonistic tyrosine mimetic, any salt or ester thereof, any multimeric and/or polymeric form of the tyrosine residue and/or of said mTOR agonistic tyrosine mimetic, and any combinations or mixtures thereof, optionally, in a first dosage form; (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof, optionally, in a second dosage form; and (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof, optionally, in a third dosage form. It should be understood that in some embodiments all three aromatic amino acid residues may be formulated in a single dosage form. In yet some further embodiments, the three aromatic amino acid residues used by the methods of the present disclosure may be formulated in one, two or three dosage forms. It should be noted that in case the subject is treated with a combination comprising at least two of the mTOR agonist/s of the invention, and/or in cases where the treatment is further combined with other agents, e.g., at least one UPS-modulating agent, for example, at least one proteasome inhibitor, or any other compound that modulates directly or indirectly at least one of the levels, stability and bioavailability of the at least one aromatic amino acid residue used herein as mTOR agonist/s, the various therapeutic compounds may be administered either together in a single composition or administration mode, or alternatively, in separate compositions, and/or different administration modes.

As discussed herein, for treating conditions associated with short term stress processes, the subject is administered with at least one aromatic amino acid residue, specifically, at least one of tyrosine, tryptophan and phenylalanine, or any mimetics thereof, or any other mTOR agonist/s. In some embodiments, the methods result in increasing at least one of tyrosine, tryptophan, and/or phenylalanine levels, beyond the endogenous level of such amino acid available in cells after ingesting a dietary source of the amino acid. In some embodiments, the levels of at least one of tyrosine, tryptophan, and/or phenylalanine are increased to at least 1.1 to at least 10 or more fold than the endogenous level available in cells after ingesting a dietary source of that amino acid, specifically, at least 1.1, at least 1.2, at least 1.3, at least 1.4, at least 1.5, at least 1.6, at least 1.7, at least 1.8, at least 1.9, at least 2.0 or more, at least 3.0 or more, at least 4.0 or more, at least 5.0 or more, at least 6.0 or more, at least 7.0 or more, at least 8.0 or more, at least 9.0 or more, at least 10 or more fold than the endogenous level available in cells after ingesting a dietary source of that amino acid. In some embodiments, where an mTOR agonistic tryptophan mimetic, an mTOR agonistic tyrosine mimetic, and/or an mTOR agonistic phenylalanine mimetic is delivered to the cell, the sum of the levels of tyrosine, tryptophan, and/or phenylalanine, and/or the corresponding mTOR amino acid mimetic is at least 1.1 to at least 10 or more fold than the endogenous level available in cells after ingesting a dietary source of that amino acid, specifically, at least 1.1, at least 1.2, at least 1.3, at least 1.4. at least 1.5, at least 1.6, at least 1.7. at least 1.8, at least 1.9, at least 2.0 or more, at least 3.0 or more, at least 4.0 or more, at least 5.0 or more, at least 6.0 or more, at least 7.0 or more, at least 8.0 or more, at least 9.0 or more, at least 10 or more fold than the endogenous level of at least one of the tyrosine, tryptophan and/or phenylalanine available in cells after ingesting a dietary source of that amino acid. It should be understood that to achieve the aforementioned levels of at least one of the tyrosine, tryptophan, phenylalanine, or a corresponding mTOR agonistic mimetic thereof, these agonist/s can be delivered in the form of either one or more single amino acid or mTOR mimetics thereof, or one or more peptides, non-standard peptides, polypeptides, non-standard polypeptides, proteins or non-standard proteins enriched for one or more those amino acids or mTOR mimetics, or any dosage form thereof or composition thereof as discussed herein before.

In some embodiments, the therapeutic methods of the invention may be further applicable to subject that are further administered with at least one UPS-modulating agent, for example, at least one proteasome inhibitor prior to, after and/or simultaneously with administration of the at least one mTOR agonist. Thus, according to some embodiments, the methods of the invention may further encompass administering to the treated subject at least one UPS-modulating agent, for example, at least one proteasome inhibitor, and/or PROTAC, prior to, after and/or simultaneously with administration of the at least one mTOR agonist. In some embodiments, the subject may be further administered with at least one additional therapeutic agent, for example, at least one agent enhancing a stress condition or process or cytosolic proteasomal localization and/or activity. For example, agents that leads to, enhances, and/or aggravates hypoxia. In some specific embodiments, such agents may inhibit or reduce angiogenesis. Non-limiting examples of angiogenesis inhibitors useful in the methods, compositions and kits of the present disclosure include at least one of: VEGF inhibitors, for example, anti-VEGF antibodies or VEGF fusion proteins, kinase inhibitors and agents involved with degradation of proteins. Still further, in some alternative and non-limiting embodiments, the treated subject may be further subjected to a restrictive diet. In some embodiments, at least one of the mTOR agonists used by the methods of the present disclosure may be formulated as any suitable dosage unit form. In some embodiments, the at least one mTOR agonists used by the methods of the present disclosure may be formulated as an oral dosage form. In yet some alternative embodiments, the at least one mTOR agonist of the methods disclosed herein may be formulated as an injectable dosage form.

In some further embodiments, the oral dosage form may be administered by the methods of the present disclosure orally, for example, as a solution (e.g., syrup), as a powder, tablet, capsule, and the like. In yet some further embodiments, the oral dosage form may be provided in a formulation adapted for add-on to a solid, semi-solid or liquid food, beverage, food additive, food supplement, medical food, drug and/or a pharmaceutical composition, as discussed herein before in connection with other aspects of the invention. As such, the oral dosage form may be part of the meal or beverage provided to the treated subject.

Thus, in some embodiments, the method disclosed herein involves oral administration, where the at least one mTOR agonist is administered orally to the treated subject. Still further, it should be understood that the methods of the present disclosure may use any of the systematic and non-systematic, or local administration modes, as well as any of the formulations and compositions adapted for any of the administration modes disclosed by the present disclosure, as discussed in connection with other aspects of the invention.

In some embodiments, the subject treated by the present disclosure is and/or was subjected to dietary restriction of amino acids, that may be also referred to herein as amino acid starvation condition. Dietary restriction of at least one amino acid as used herein, is meant the provision of a controlled diet regimen to the treated subject, that includes no protein source or a very low protein content. In some embodiments, the dietary restriction of amino acids, involves the provision of a diet regimen characterized in depletion, or restriction of either all 20 amino acids, or at least the essential amino acids, for example, at least one of, phenylalanine, valine, threonine, tryptophan, methionine, leucine, isoleucine, lysine, and histidine. Still further, a balanced diet of adults requires consumption of at least 10% of the daily calories in the form of protein. A low protein content, or no protein content of a diet regimen, is meant any diet that provides the subject treated by the methods of the present disclosure, less than the required protein amount, for example, between about 0 to about 5%, of the required daily protein amount, specifically, 0, 0.001, 0.002, 0.003, 0.004, 0.005, 0.006, 0.007, 0.008, 0.009, 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.100, 0.1, 0.2, 0.3, 0.4, 0.5, 0.6, 0.7, 0.8, 0.9, 1, 2, 3, 4, or 5% of the required daily protein amount.

It should be understood that in some embodiments, the methods of the present disclosure may comprise a further step, of subjecting the subject to amino acid starvation, depletion, depravation or restriction as discussed herein above. This step may be performed in accordance with some embodiments, either before, with, or after the administration of the mTOR agonists of the invention or any dosage form or compositions thereof, or any meal or beverage comprising the same. In some particular and non-limiting embodiments, the methods disclosed herein comprise the administration of all three aromatic amino acid residues Y, W, F, in an effective amount as disclosed herein above in connection with other aspects of the invention. More specifically, in some embodiments, the methods disclosed herein comprise the administration of the aromatic amino acids Y, W and F, in a concentration ranging between about 0.01 mM to about 30 mM or more, provided that the concentration of each of the aromatic amino acid residues is less than 45 mM, and in some further embodiments, the concentration is no more than 35 mM, as discussed in connection with other aspects of the present disclosure. In yet some further embodiment, the methods disclosed herein comprise the administration of an amount of between about 5 gr-7 gr, to about 50 gr-70 gr of each of the aromatic amino acid residues Y, W, F. In yet some further embodiments, the effective amount used and administered by the methods disclosed herein may range between about 0.1 gr per day/per kg to about 0.9 gr per day/per kg, for each of the aromatic amino acid residues Y, W, F, and in some embodiments, no more than 0.99 gr per day/per kg, for each of the aromatic amino acid residues Y,W, F. It should be understood however that the indicated effective doses per day, or dosage unit as discussed herein, may be given either in a single administration or in two or more administrations at several time-points over 24 hr. Still further, administration and doses are determined by good medical practice of the attending physician and may depend on the age, sex, weight and general condition of the subject in need.

In some embodiments, the method of the invention may be applicable for pathologic disorder associated with cytosolic proteasomal localization and/or activity, and/or disorder involved with at least one short term cellular stress condition/process is at least one of proliferative disorder and/or at least one protein misfolding disorder or deposition disorder.

In more specific embodiments, the proliferative disorder may be at least one benign or malignant solid and non-solid tumor. In yet some further embodiments, the protein misfolding disorder is amyloidosis and any related conditions.

Still further, the mTOR agonists, compositions and kits of the present disclosure may be applicable for any proliferative disorder that may be in some embodiments, any neoplastic disease, more specifically, any abnormal mass of tissue, also referred to herein as a tumor, that is formed due to uncontrolled or abnormal cell growth that results increased cell number. The methods of the present disclosure may be applicable in some embodiments for any neoplasms, either benign neoplasms, in situ neoplasms, or malignant neoplasms.

In some embodiments, the methods of the invention may be applicable for treating adenomas. More specifically, adenoma is a benign tumor of epithelial tissue with glandular origin, glandular characteristics, or both. Adenomas can grow from many glandular organs, including the adrenal glands, pituitary gland, thyroid, prostate, and others. Although adenomas are benign, they should be treated as pre-cancerous. Over time adenomas may transform to become malignant, at which point they are called adenocarcinomas. It should be understood that the present invention is further applicable to any metastatic tissue, organ or cavity of any of the disclosed proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions and kits of the present invention may be applicable for a patient suffering from any one of non-solid and solid tumors.

Malignancy, as contemplated in the present invention may be any one of carcinomas, melanomas, lymphomas, leukemia, myeloma and sarcomas. Therefore, in some embodiments any of the methods of the invention (specifically, therapeutic, prognostic and non-therapeutic methods), kits and compositions disclosed herein, may be applicable for any of the malignancies disclosed by the present disclosure.

More specifically, carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

In some embodiments, the methods of the present disclosure may be applicable for any solid tumor. In more specific embodiments, the methods disclosed herein may be applicable for any malignancy that may affect any organ or tissue in any body cavity, for example, the peritoneal cavity (e.g., liposarcoma), the pleural cavity (e.g., mesothelioma, invading lung), any tumor in distinct organs, for example, the urinary bladder, ovary carcinomas, and tumors of the brain meninges. Particular and non-limiting embodiments of tumors applicable in the methods, compositions and kit of the present disclosure may include but are not limited to at least one of ovarian cancer, liver carcinoma, colorectal carcinoma, breast cancer, pancreatic cancer, brain tumors and any related conditions, as well as any metastatic condition, tissue or organ thereof.

In some other embodiments, the methods, compositions and kits of the invention are applicable to colorectal carcinoma, or any malignancy that may affect all organs in the peritoneal cavity, such as liposarcoma for example. In some further embodiments, the method of the invention may be relevant to tumors present in the pleural cavity (mesothelioma, invading lung) the urinary bladder, and tumors of the brain meninges.

In some particular embodiments, the methods, compositions and kits of the invention may be applicable for ovarian cancer. It should be further understood that the invention further encompasses any tissue, organ or cavity barring ovarian metastasis, as well as any cancerous condition involving metastasis in ovarian tissue. As used herein, the term “ovarian cancer” is used herein interchangeably with the term “fallopian tube cancer” or “primary peritoneal cancer” referring to a cancer that develops from ovary tissue, fallopian tube tissue or from the peritoneal lining tissue. Early symptoms can include bloating, abdominopelvic pain, and pain in the side. The most typical symptoms of ovarian cancer include bloating, abdominal or pelvic pain or discomfort, back pain, irregular menstruation or postmenopausal vaginal bleeding, pain or bleeding after or during sexual intercourse, difficulty eating, loss of appetite, fatigue, diarrhea, indigestion, heartburn, constipation, nausea, early satiety, and possibly urinary symptoms (including frequent urination and urgent urination). Typically, these symptoms are caused by a mass pressing on the other abdominopelvic organs or from metastases.

The most common type of ovarian cancer, comprising more than 95% of cases, is epithelial ovarian carcinoma. These tumors are believed to start in the cells covering the ovaries, and a large proportion may form at end of the fallopian tubes. Less common types of ovarian cancer include germ cell tumors and sex cord stromal tumors. Ovarian cancers are classified according to the microscopic appearance of their structures (histology or histopathology).

Surface epithelial-stromal tumor, also known as ovarian epithelial carcinoma, is the most common type of ovarian cancer, representing approximately 90% of ovarian cancers. It includes serous tumor, endometrioid tumor, clear cell tumor, and mucinous cystadenocarcinoma. Less common tumors are malignant Brenner tumor and transitional cell carcinoma of the ovary. Low-grade serous carcinoma is less aggressive than high-grade serous carcinomas, though it does not typically respond well to chemotherapy or hormonal treatments.

Small-cell ovarian carcinoma is rare and aggressive, with two main subtypes: hypercalcemic and pulmonary. Hypercalcemic small cell ovarian carcinoma overwhelmingly affects those in their 20s, causes high blood calcium levels, and affects one ovary. Pulmonary small cell ovarian cancer usually affects both ovaries of older women and looks like oat-cell carcinoma of the lung.

Primary peritoneal carcinoma develops from the peritoneum. It can develop even after the ovaries have been removed and may appear similar to mesothelioma.

Clear-cell ovarian carcinomas may be related to endometriosis. They represent approximately 5-10% of epithelial ovarian cancers and are associated with endometriosis in the pelvic cavity.

Endometrioid adenocarcinomas make up approximately 15-20% of epithelial ovarian cancers.

These tumors frequently co-occur with endometriosis or endometrial cancer.

Mixed mullerian tumors make up less than 1% of ovarian cancer. They have epithelial and mesenchymal cells visible.

Mucinous tumors include mucinous adenocarcinoma and mucinous cystadenocarcinoma. Mucinous adenocarcinomas make up 5-10% of epithelial ovarian cancers.

Pseudomyxoma peritonei refers to a collection of encapsulated mucous or gelatinous material in the abdominopelvic cavity, which is very rarely caused by a primary mucinous ovarian tumor.

Malignant Brenner tumors are rare. Histologically, they have dense fibrous stroma with areas of transitional epithelium, and some squamous differentiation. To be classified as a malignant Brenner tumor, it must have Brenner tumor foci and transitional cell carcinoma. The transitional cell carcinoma component is typically poorly differentiated and resembles urinary tract cancer.

Sex cord-stromal tumor, including estrogen-producing granulosa cell tumor, the benign thecoma, and virilizing Sertoli-Leydig cell tumor or arrhenoblastoma, accounts for 7% of ovarian cancers. Granulosa cell tumors are the most common sex-cord stromal tumors, making up 70% of cases, and are divided into two histologic subtypes: adult granulosa cell tumors, which develop in women over 50, and juvenile granulosa tumors, which develop before puberty or before the age of 30. Both develop in the ovarian follicle from a population of cells that surrounds germinal cells.

Germ cell tumors of the ovary develop from the ovarian germ cells. Germ cell tumor accounts for about 30% of ovarian tumors, but only 5% of ovarian cancers, because most germ-cell tumors are teratomas and most teratomas are benign. Malignant teratomas tend to occur in older women, when one of the germ layers in the tumor develops into a squamous cell carcinoma. Germ-cell tumors can include dysgerminomas, teratomas, yolk sac tumors/endodermal sinus tumors, and choriocarcinomas, when they arise in the ovary.

It should be appreciated that ovarian carcinoma as used herein may further include at least one of, Ovarian carcinosarcoma. Choriocarcinoma, Mature teratomas, Embryonal carcinomas and Primary ovarian squamous cell carcinomas. More specifically, Ovarian carcinosarcoma (OCS), also known as malignant mixed mullerian tumor (MMMT), is a very rare gynecological malignancy accounting for 1-3% of ovarian malignancies. OCS is a mixed tumor composed of sarcomatous and carcinomatous components. The sarcomatous component may be either homologous, including endometrial stromal sarcoma. fibrosarcoma and leiomyosarcoma. or heterologous. The carcinomatous component often consists of adenocarcinoma, and squamous cell carcinoma. Because women with this cancer often have no symptoms, more than half of women are diagnosed at an advanced stage. When present. symptoms may include pain in the abdomen or pelvic area, bloating or swelling of the abdomen, quickly feeling full when eating, or other digestive problems. Choriocarcinoma, can occur as a primary ovarian tumor developing from a germ cell, though it is usually a gestational disease that metastasizes to the ovary. Mature teratomas, or dermoid cysts, are rare tumors consisting of mostly benign tissue that develop after menopause. Embryonal carcinomas, a rare tumor type usually found in mixed tumors, develop directly from germ cells but are not terminally differentiated; in rare cases they may develop in dysgenetic gonads. They can develop further into a variety of other neoplasms, including choriocarcinoma, yolk sac tumor, and teratoma. Primary ovarian squamous cell carcinomas are rare and have a poor prognosis when advanced. More typically, ovarian squamous cell carcinomas are cervical metastases, areas of differentiation in an endometrioid tumor, or derived from a mature teratoma.

In yet some other embodiments, the methods, kits and compositions of the present disclosure may be suitable for liver cancer. It should be further understood that the invention further encompasses any tissue, organ or cavity barring liver originated metastasis, as well as any cancerous condition having metastasis of any origin in liver tissue. Liver cancer, also known as hepatic cancer and primary hepatic cancer, is cancer that starts in the liver. Cancer which has spread from elsewhere to the liver, known as liver metastasis, is more common than that which starts in the liver. Symptoms of liver cancer may include a lump or pain in the right side below the rib cage, swelling of the abdomen, yellowish skin, easy bruising, weight loss and weakness.

The leading cause of liver cancer is cirrhosis due to hepatitis B, hepatitis C or alcohol. Other causes include aflatoxin, non-alcoholic fatty liver disease and liver flukes. The most common types are hepatocellular carcinoma (HCC), which makes up 80% of cases, and cholangiocarcinoma. Less common types include mucinous cystic neoplasm and intraductal papillary biliary neoplasm. The diagnosis may be supported by blood tests and medical imaging, with confirmation by tissue biopsy. As used herein, HCC, is the most common type of primary liver cancer in adults, and is the most common cause of death in people with cirrhosis. It occurs in the setting of chronic liver inflammation and is most closely linked to chronic viral hepatitis infection (hepatitis B or C) or exposure to toxins such as alcohol or aflatoxin. Certain diseases, such as hemochromatosis, Diabetes mellitus and alpha 1-antitrypsin deficiency, markedly increase the risk of developing HCC. Metabolic syndrome and NASH are also increasingly recognized as risk factors for HCC.

Cholangiocarcinoma, also known as bile duct cancer, is a type of cancer that forms in the bile ducts. Symptoms of cholangiocarcinoma may include abdominal pain, yellowish skin, weight loss, generalized itching, and fever. Light colored stool or dark urine may also occur. Other biliary tract cancers include gallbladder cancer and cancer of the ampulla of Vater. Risk factors for cholangiocarcinoma include primary sclerosing cholangitis (an inflammatory disease of the bile ducts), ulcerative colitis, cirrhosis, hepatitis C, hepatitis B, infection with certain liver flukes, and some congenital liver malformations. The diagnosis is suspected based on a combination of blood tests, medical imaging, endoscopy, and sometimes surgical exploration. The disease is confirmed by examination of cells from the tumor under a microscope. It is typically an adenocarcinoma (a cancer that forms glands or secretes mucin).

In other embodiments, the methods, kits and compositions of the present disclosure may be applicable for pancreatic cancer. It should be further understood that the invention further encompasses any tissue, organ or cavity barring pancreatic metastasis, as well as any cancerous condition having metastasis of any origin in the pancreas. Pancreatic cancer arises when cells in the pancreas, a glandular organ behind the stomach, begin to multiply out of control and form a mass. There are a number of types of pancreatic cancer. The most common, pancreatic adenocarcinoma, accounts for about 90% of cases. These adenocarcinomas start within the part of the pancreas which makes digestive enzymes. Several other types of cancer, which collectively represent the majority of the non-adenocarcinomas, can also arise from these cells. One to two percent of cases of pancreatic cancer are neuroendocrine tumors, which arise from the hormone-producing cells of the pancreas. These are generally less aggressive than pancreatic adenocarcinoma.

Signs and symptoms of the most-common form of pancreatic cancer may include yellow skin, abdominal or back pain, unexplained weight loss, light-colored stools, dark urine, and loss of appetite. There are usually no symptoms in the disease's early stages, and symptoms that are specific enough to suggest pancreatic cancer typically do not develop until the disease has reached an advanced stage. By the time of diagnosis, pancreatic cancer has often spread to other parts of the body.

Pancreatic cancer rarely occurs before the age of 40, and more than half of cases of pancreatic adenocarcinoma occur in those over 70. Risk factors for pancreatic cancer include tobacco smoking, obesity, diabetes, and certain rare genetic conditions. Pancreatic cancer is usually diagnosed by a combination of medical imaging techniques such as ultrasound or computed tomography, blood tests, and examination of tissue samples (biopsy).

It should be understood that the methods, compositions and kits of the present disclosure are applicable for any type and/or stage and/or grade of any of the malignant disorders discussed herein or any metastasis thereof. Still further, it must be appreciated that the methods, compositions and kits of the invention may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

Still further, in some embodiments, the methods, compositions and kits of the present disclosure are applicable for any type and/or stage and/or grade of any metastasis, metastatic cancer or status of any of the cancerous conditions disclosed herein.

As used herein the term “metastatic cancer” or “metastatic status” refers to a cancer that has spread from the place where it first started (primary cancer) to another place in the body. A tumor formed by metastatic cancer cells originated from primary tumors or other metastatic tumors, that spread using the blood and/or lymph systems, is referred to herein as a metastatic tumor or a metastasis. Further malignancies that may find utility in the present invention can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer, Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor, Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer, Cutaneous T-cell lymphoma; Desmoplastic small round cell tumor, Endometrial cancer, Ependymoma; Esophageal cancer, Ewing's sarcoma in the Ewing family of tumors; Extracranial germ cell tumor, Childhood; Extragonadal Germ cell tumor; Extrahepatic bile duct cancer, Eye Cancer, Intraocular melanoma; Eye Cancer, Retinoblastoma; Gallbladder cancer; Gastric (Stomach) cancer, Gastrointestinal Carcinoid Tumor, Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer, Hepatocellular (liver) cancer, Hodgkin lymphoma; Hypopharyngeal cancer, Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer, Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer, Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer, Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer, Nasopharyngeal carcinoma; Neuroblastoma; Non-Hodgkin lymphoma; Non-small cell lung cancer; Oral Cancer; Oropharyngeal cancer, Osteosarcoma/malignant fibrous histiocytoma of bone; Ovarian cancer; Ovarian epithelial cancer (Surface epithelial-stromal tumor); Ovarian germ cell tumor, Ovarian low malignant potential tumor, Pancreatic cancer; Pancreatic cancer, islet cell; Paranasal sinus and nasal cavity cancer; Parathyroid cancer, Penile cancer, Pharyngeal cancer, Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer, Renal cell carcinoma (kidney cancer); Renal pelvis and ureter, transitional cell cancer, Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer, Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer, Urethral cancer, Uterine cancer, endometrial; Uterine sarcoma; Vaginal cancer, Visual pathway and hypothalamic glioma, childhood; Vulvar cancer; Waldenstrom macroglobulinemia and Wilms tumor (kidney cancer).

In some further embodiments, the methods of the invention may further comprise administering a drug that enables increasing directly or indirectly at least one of the levels, stability and bioavailability of at least one mTOR agonist of the invention, specifically the aromatic amino acids phenylalanine, tryptophan and/or tyrosine. In some embodiments, such compound may be administered together with the at least one of the aromatic amino acid residues of the invention, specifically, phenylalanine, tryptophan and/or tyrosine. In yet some alternative embodiments this compound may be administered as a sole therapeutic or non-therapeutic compound to increase directly or indirectly at least one of the levels, stability and bioavailability of at least one mTOR agonist of the invention.

In some particular and non-limiting embodiments, such compound may be for example Nitisinone, which is an FDA-approved drug, used for Hereditary Hypertyrosinemia Type-1. More specifically, Nidsinone (INN), also known as NTBC (an abbreviation of its full chemical name) is a medication is an FDA-approved drug, used to slow the effects of Hereditary Hypertyrosinemia Type-1 (HT-1). It is used in patients from all ages, in combination with dietary restriction of tyrosine and phenylalanine. Besides elevating Tyrosine (Y) levels—via inhibition of its metabolism, the drug also increases the level of Phenylalanine (F). The mechanism of action of nitisinone involves reversible inhibition of 4-Hydroxyphenylpyruvate dioxygenase (HPPD). It prevents the formation of maleylacetoacetic acid and fumarylacetoacetic acid, which have the potential to be converted to succinyl acetone, a toxin that damages the liver and kidneys.

Nitisinone has the following chemical structure, as denoted by Formula X:

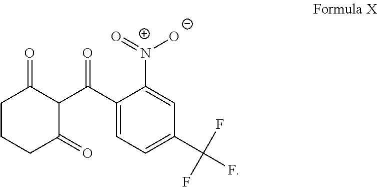 [SMILES: O=C1CCCC(=O)C1C(=O)c1ccc(C(F)(F)F)cc1[N+](=O)[O-]]

The systematic (IUPAC) name of Nitisinone is 2-[2-nitro-4-(trifluoromethyl)benzoyl]cyclohexane-1,3-dione (C₁₄H₁₀F₃NOs; CAS number: 104206-65-7). The molecular weight of the form of Nitisinone depicted above is 329.228 gram/mol.

As indicated above, the methods of the present disclosure applicable for treating any of the cancerous disorders disclosed by the invention may use any of the mTOR agonist disclosed herein that comprise at least one, a least two or all three aromatic amino acid residues, specifically, (a), at least one tyrosine residue, any mTOR agonistic tyrosine mimetic, any salt or ester thereof, any multimeric and/or polymeric form of the tyrosine residue and/or of said mTOR agonistic tyrosine mimetic, and any combinations or mixtures thereof, optionally, in a first dosage form, (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof, optionally, in a second dosage form, and (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof, optionally, in a third dosage form. It should be understood that in some embodiments, any derivative or any mimetic form of any of the aromatic amino acid residues disclosed herein may be used by the present agonists, compositions, kits, methods and uses of the present disclosure. However, in some embodiments, any derivative may be used herein provided that said derivative is not a fluorinated aromatic amino acid residue. In some embodiments, any tryptophan derivative may be used by the mTOR agonists, compositions, kits and methods of the present disclosure provided that the tryptophan derivative is not a fluorinated tryptophane, more specifically, L-(4-F)-Trp. In yet some further specific embodiments, any tryptophan derivative may be used by the mTOR agonists, compositions, kits and methods of the present disclosure provided that the tryptophan derivative is not a fluorinated tryptophane, specifically, any one of (6-F)-Trp or (5-F)-Trp. In yet some further embodiments, any tyrosine derivative may be used by the mTOR agonists, compositions, kits and methods of the present disclosure provided that the tyrosine derivative is not a fluorinated tyrosine, specifically, m-FTyr. Still further, in some embodiments, any phenylalanine derivative may be used by the selective inhibitors of proteasome translocation and/or mTOR agonists, compositions, kits and methods of the present disclosure provided that the phenylalanine derivative is not a fluorinated phenylalanine, specifically, any one of o-FPhe, m-FPhe, o p-FPhe.

In yet some further embodiments, any aromatic amino acid derivative may be used by the mTOR agonists, compositions, kits and methods of the present disclosure provided that said derivative is not 3, 5-Dichloro-O-[(2-phenyl)-benzoxazo1-7-yl] methyl-L-tyrosine methyl ester hydrochloride. Still further, any aromatic amino acid derivative may be used by the mTOR agonists, compositions, kits and methods of the present disclosure provided that said derivative is not 3-(2-naphthyloxy)-L-phenylalanine. It should be understood that the proviso discussed herein may be applicable in some embodiments to any of the aspects of the present disclosure, specifically, to any one the mTOR agonists, compositions, kits and methods of the present disclosure.

A further aspect of the invention relates to an effective amount, or in some embodiments, a therapeutically effective amount of at least one mTOR agonist for use in a method for treating, preventing, inhibiting, reducing, eliminating, protecting or delaying the onset of at least one pathologic disorder involved with at least one short term cellular stress condition/process. More specifically, any of the mTOR agonist/s used herein may comprise at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compound that modulates directly or indirectly at least one of the levels, stability and bioavailability of the at least one aromatic amino acid residue, any combinations or mixtures thereof, any dosage forms thereof, any composition or kit comprising the at least one mTOR agonist of the present disclosure.

As discussed above, the mTOR agonist/s detailed above in the context of the previously mentioned methods, compositions and kits of the invention are relevant for use in a method for treating, preventing, inhibiting, reducing, eliminating, protecting or delaying the onset of at least one pathologic disorder involved with at least one short term cellular stress condition/process.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease. The term “treatment or prevention” as used herein, refers to the complete range of therapeutically positive effects of administrating to a subject including inhibition, reduction of, alleviation of, and relief from, pathologic disorder involved with at least one short term cellular stress condition/process and any associated condition, illness, symptoms, undesired side effects or related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the at least one short term cellular stress condition/process and their symptoms, slowing their progress, further exacerbation or development, so as to appear later than in the absence of the treatment according to the invention.

As indicated above, the methods and compositions provided by the present invention may be used for the treatment of a “pathological disorder”, i.e., pathologic disorder or condition involved with at least one short term cellular stress condition/process, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods, kits and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

In yet some further aspects thereof, the present discloser provides in vivo and in vitro modulatory methods having further therapeutic and non-therapeutic applications. The non-therapeutic applications of such modulatory methods may encompass cosmetic and agricultural uses of the mTOR agonist/s of the invention.

More specifically, in a further aspect thereof, the present disclosure relates to a method for modulating a biological process associated directly or indirectly with proteasome dynamics in at least one cell and/or a subject. According to some embodiments, the methods comprise the step of contacting the at least one cell and/or administering to the subject a therapeutically effective amount of at least one mTOR agonist comprising at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compounds that modulate directly or indirectly at least one of the levels, stability and bioavailability of the at least one aromatic amino acid residue, any combinations or mixtures thereof, any vehicle, matrix, nano- or micro-particle thereof, any combinations or mixtures thereof, any vehicle, matrix, nano- or micro-particle thereof, any dosage forms thereof or any composition or kit comprising the at least one mTOR agonist of the present disclosure.

In yet some more specific embodiments, the mTOR agonist used by the methods provided by the present disclosure, may comprise at least one aromatic amino acid residue or a combination of at least two aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In some specific embodiments, the mTOR agonist/s of the methods disclosed herein may comprise at least one of the following components. First component (a), comprises at least one tyrosine (Y) residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof, optionally, in a dosage form. The mTOR agonist may comprise in some embodiments alternatively or additionally, as a second component (b), at least one tryptophan (W) residue, any mTOR agonistic tryptophan mimetic, any salt or ester thereof, any multimeric and/or polymeric form of the tryptophan residue and/or of the mTOR agonistic tryptophan mimetic, or any combination or mixture thereof, optionally, in a dosage form. In yet some further embodiments, the mTOR agonist of the invention may comprise alternatively, or additionally, as a third component (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof, optionally, in at least one dosage form. It should be appreciated that any combination of Y and W, or Y and F, or W and F, are also encompassed by the disclosed methods.

Still further, in some specific embodiments, the mTOR agonist used by the methods of the present disclosure may comprise a combination of the following three components: (a), at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist/s of the invention further comprise (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof. The mTOR agonist of the methods of the present disclosure further comprises (c), at least one phenylalanine residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof. It should be noted that in some embodiments, the at least one, two or all three aromatic amino acid residues Y, W, F, or any mimetics thereof and any combinations thereof, may be used by the methods of the invention when formulated in one or more dosage unit forms.

In some embodiments, where the method of the present disclosure involves modulating a biological process associated directly or indirectly with proteasome dynamics in at least one cell, the at least one cell may be subjected to, or may undergo amino acid deprivation, amino acid starvation, amino acid depletion, amino acid depravation, or amino acid restriction. More specifically, the cell may be provided with a growth medium with no, or with a minimal amount of all 20 amino acids.

In yet some other embodiments, where the method of the present disclosure involves modulating a biological process associated directly or indirectly with proteasome dynamics in a subject, the treated subject may be and/or was subjected to dietary restriction of amino acids, specifically, depletion, or restriction of either all 20 amino acids, or at least the essential amino acids, for example, at least one of, phenylalanine, valine, threonine, tryptophan, methionine, leucine, isoleucine, lysine, and histidine. As specified above, the treated subject may be provided with a low or no protein diet regimen.

As shown by the following Examples, modulation of proteasome dynamics in cells by the mTOR agonist/s of the invention, control cell survival, and can be used successfully for cancer therapy. However, such modulatory effects may be also used for modulating and altering muscle mass. Thus, in the current aspect, the present invention provides at least one mTOR agonist, specifically, at least one aromatic amino acid residue or any combinations thereof, specifically, at least one of Y, W, and/or F, for modulating proteasome dynamics by increasing mTOR activity in a cell and/or a subject. Such modulation is used herein to increase muscle mass, increase muscle anabolism, or to treat a disease or condition that involves skeletal muscle atrophy. In this connection, muscular wasting or atrophy may be either genetic or induced by a systemic disease, like cancer or sepsis or renal insufficiency.

More specifically, in some embodiments the at least one aromatic amino acid residue or any combinations thereof disclosed herein, may be used in the methods disclosed herein to promote muscle anabolism. improve muscle function, increase muscle mass, reverse muscle atrophy or to prevent muscle atrophy. In some embodiments, the mTOR agonist/s of the invention may be applicable in therapeutic methods for disorder/s characterized by muscle atrophy that may be any one of aging, bony fractures, weakness, cachexia, denervation, diabetes, dystrophy, exercise-induced skeletal muscle fatigue, fatigue, frailty, immobilization, inflammatory myositis, malnutrition, metabolic syndrome, neuromuscular disease, obesity, post-surgical muscle weakness, post-traumatic muscle weakness, sarcopenia, and toxin exposure. In some embodiments, the methods of the invention may be used to reverse muscle atrophy or to prevent muscle atrophy due to inactivity, immobilization, or age of the subject or a disease or condition suffered by the subject. In some embodiments, the methods of the present disclosure may be used to reverse muscle atrophy or to prevent muscle atrophy due to a broken bone, a severe burn, a spinal injury, an amputation, a degenerative disease, a condition wherein recovery requires bed rest for the subject, a stay in an intensive care unit, or long-term hospitalization. The term “bed rest” as used herein means that the subject is confined or required by a doctor to remain in bed, sitting and/or lying down for at least 80% of the day for at least 3 days. The term “long-term hospitalization” as used herein means a stay in a hospital or other health care facility for at least five days.

Still further, the methods of the invention may be applicable for preventing or reversing cardiac muscle atrophy (e.g., where a subject is suffering from or has suffered from heart attack, congestive heart failure, heart transplant, heart valve repair, atherosclerosis, other major blood vessel or ischemic disease, and heart bypass surgery.

In yet some further embodiments of the methods disclosed herein, the subject is suffering from a disease or condition known to be associated with cachexia for example, from cancer, viral infections, specifically, AIDS (HIV infection), SARS (SARS CoV infection), and COVID 19 (SARS CoV2 infection), chronic heart failure. COPD, rheumatoid arthritis, liver disease, kidney disease and trauma. In some embodiments, the subject is suffering from a disease or condition known to be associated with malabsorption. In some embodiments, such malabsorption the disease or condition may be any one of Crohn's disease, irritable bowel syndrome, celiac disease, and cystic fibrosis. In some embodiments, the methods of the present disclosure are applicable for subjects suffering from malnutrition, sarcopenia, muscle denervation, muscular dystrophy, an inflammatory myopathy, Spinal Muscle Atrophy, ALS, or myasthenia gravis.

More specifically, Muscular atrophy is the loss of skeletal muscle mass that can be caused by immobility, aging, malnutrition, medications, or a wide range of injuries or diseases that impact the musculoskeletal or nervous system. Muscle atrophy leads to muscle weakness and causes disability. Disuse causes rapid muscle atrophy and often occurs during injury or illness that requires immobilization of a limb or bed rest. Depending on the duration of disuse and the health of the individual, this may be fully reversed with activity. Malnutrition first causes fat loss but may progress to muscle atrophy in prolonged starvation and can be reversed with nutritional therapy. In contrast, cachexia is a wasting syndrome caused by an underlying disease such as cancer that causes dramatic muscle atrophy and cannot be completely reversed with nutritional therapy. Sarcopenia is the muscle atrophy associated with aging and can be slowed by exercise. Finally, diseases of the muscles such as muscular dystrophy or myopathies can cause atrophy, as well as damage to the nervous system such as in spinal cord injury or stroke.

Muscle atrophy results from an imbalance between protein synthesis and protein degradation, although the mechanisms are variable depending on the cause. Muscle loss can be quantified with advanced imaging studies. Treatment depends on the underlying cause but will often include exercise and adequate nutrition. Anabolic agents may have some efficacy but are not often used due to side effects. Still further, in some embodiments, a subject suffering from a disorder, condition, or symptom associated with muscle atrophy is a subject whose skeletal muscle mass has decreased by at least a 5% as a result of the disorder, condition, or symptom. In some embodiments, such subject may display a decrease in the skeletal muscle mass of at least about 5%. 8%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more as a result of the disorder, condition, or symptom. In some embodiments, a subject suffering from a disorder, condition, or symptom associated with muscle atrophy is a subject whose muscle weight relative to body weight ratio decreased by at least a 2%, at least a 3%, at least a 4%, at least a 5%, at least 6%, at least 7%, at least 8%, at least 9%, at least 10%, at least a 15%, at least a 16%, at least a 20%, at least a 25%. at least 30%, at least 35%, or at least 40% or more as a result of the disorder, condition, or symptom.

In some embodiments, any of the methods of increasing mTOR activation/activity and thereby, increasing proteasome nuclear localization set forth herein, can be used for increasing skeletal muscle mass. Still further, as used herein, “increasing skeletal muscle mass” refers to a statistically significant increase in the skeletal muscle mass. In some embodiments of various aspects, increasing skeletal muscle mass refers to a reversal of skeletal muscle loss. In some embodiments of various aspects, increasing skeletal muscle mass refers to an increase in skeletal muscle mass of at least 5%. at least 7%, at least 12%, at least 15%, at least 18%, at least 20%, at least 21%, at least 25%, at least 27%, at least 30%, at least 33% or more, relative to the skeletal muscle mass prior to contacting the skeletal muscle with the mTOR agonist/s of the invention, specifically, at least one of tyrosine, tryptophan, and/or phenylalanine, any mimetics or composition thereof, and/or to administration to the subject. In some embodiments, increasing skeletal muscle mass refers to an increase in skeletal muscle mass of a subject to within 35%, within 33%, within 30%, within 28%. within 24%, within 22%, within 18%, within 15%, within 12%. within 10%, within 9%, within 8%, within 7%, within 6%, within at least 5% or more of the skeletal muscle before onset of the disorder, condition, or symptom associated with muscle atrophy, or onset of the muscle atrophy itself.

The disclosure thus provides therapeutic and non-therapeutic methods of increasing skeletal muscle mass, comprising contacting skeletal muscle or skeletal muscle cells with the at least one mTOR agonist/s of the invention, specifically, at least one of tyrosine, tryptophan and/or phenylalanine, or any mimetics, combinations and compositions thereof.

In some embodiments, the mTOR agonist/s of the invention stimulate mTOR activation and the associated proteasome nuclear localization in the skeletal muscle or skeletal muscle cells, thereby promoting skeletal muscle anabolism and increasing skeletal muscle mass.

In yet some further embodiments, the disclosure provides a method of increasing skeletal muscle mass in a subject, comprising administering to the subject an effective amount of any one of the mTOR agonist/s of the invention, specifically, at least one of tyrosine, tryptophan and/or phenylalanine or any mimetics thereof, or an effective amount of a composition comprising at least one of tyrosine, tryptophan and/or phenylalanine or any mimetics thereof, optionally, in at least one dosage form. In some embodiments, the at least one of tyrosine, tryptophan and/or phenylalanine or any mimetics thereof stimulate mTOR activation and the associated proteasome nuclear localization in the subject, thereby promoting skeletal muscle anabolism and increasing the subject's skeletal muscle mass.

As indicated herein, the method of increasing skeletal muscle mass may lead to an increase in muscle-to-fat ratio. The methods disclosed herein may therefore have additional and non-therapeutic applications, for example, cosmetic and/or agricultural uses.

More specifically, in some embodiments, the method of increasing skeletal muscle mass is used for agricultural purpose, specifically, to increase skeletal muscle mass (or increase the muscle-to fat ratio) in a non-human animal, such as livestock, fish, poultry or insects. In these embodiments, each of the mTOR agonist/s of the invention, specifically, at least one aromatic amino acid residues, more specifically, at least one of Y, W and/or F, and any mimetics thereof, may be administered as an additive to the feed of the non-human animal, used as pets and in food industry. The term “non-human animal” as used herein includes any organism, specifically all vertebrates, any non-mammal organism (e.g., fish, chickens, amphibians, reptiles and insects) and mammals, such as non-human primates, domesticated and/or agriculturally useful animals, e.g., sheep, dog, cat, cow, pig, etc. The term “livestock”, as used herein refers to any farmed animal. Preferably, livestock is one or more of ruminants such as cattle (e.g.. cows or bulls (including calves)), mono-gastric animals such as poultry (including broilers, chickens and turkeys), pigs (including piglets), birds, or sheep (including lambs).

As discussed above, in some embodiments, the present disclosure further provides cosmetic non-therapeutic methods. For example, the method of the invention, using mTOR agonist/s that modulate proteasome dynamics and lead to predominant proteasome nuclear localization, may be used for increasing muscle mass in subjects interested in such cosmetic intervention or procedure. In some embodiments, the aromatic amino acid residue/s provided herein, and any combinations thereof may be used to increase strength and/or to increase muscle mass, optionally, following exercise. In this connection, according to some embodiments of the methods discussed herein, each of the amino acid residues may be present in a beverage or a nutrition bar or any add-on composition as discussed above, that may be consumed by the subject.

In some particular embodiments of the therapeutic or non-therapeutic methods of the invention, the mTOR agonist/s of the invention, specifically, any one of the aromatic amino acid residues, Y, W, and/or F, may be administered to a subject depleted or starved to these specific amino acid residues. In some embodiments, such subject may be a fasted subject. In yet some alternative embodiments, the mTOR agonist/s of the invention, specifically, any one of the aromatic amino acid residues, Y, W, and/or F, may be administered to a subject not depleted or starved for these specific amino acid residues. For example, a fed subject. The term “fasted subject” as used herein means a subject who has not ingested a meal within a period of 1, 2, 3, 4, 5, 6, 7 or 8 hours prior to being administered at least one or all of the mTOR agonist/s utilized in the methods of this invention. In certain embodiments, the “fasted subject” also does not ingest a meal for a period of 1, 2, 3, 4, 5, 6, 7 or 8 hours after being administered the last of the mTOR agonist/s utilized in the methods of the present disclosure.

It should be appreciated that the present disclosure further encompasses methods, compositions and kits for modulating proteasome dynamics in a cell, and/or in a subject in need thereof. Thus, the present disclosure further encompasses modulatory methods that may be performed in vivo, in vitro or ex vivo. In some embodiments, the method of the present disclosure may comprise the step of contacting the cell, or at least one cell in a subject, with a modulatory effective amount of the mTOR agonist/s of the invention or any composition, combinations or kits thereof. As used herein “modulating” means causing or facilitating a qualitative or quantitative change, alteration, or modification in a molecule, a process, pathway, or phenomenon of interest. For example, cellular localization of the proteasome. Without limitation, such change may be an increase, decrease, a change in nuclear or cytosolic proteasome localization characteristics, or change in relative strength or activity of different components or branches of the process, pathway, or phenomenon.

In yet some further embodiments, the mTOR agonist/s of the invention as well as any combinations, compositions, kits and methods thereof, increase proteasome nuclear localization in a cell. As used herein “increasing”, “increased”, “increase”, “stimulate”, “enhance” or “activate” are all used herein to generally mean an increase by a statistically significant amount; for the avoidance of any doubt, the terms “increased”, “increase”, “stimulate”, “enhance” or “activate” means an increase of at least 10% as compared to a reference level of the proteasome nuclear localization. For example an increase of at least about 20%, or at least about 30%, or at least about 40%, or at least about 50%. or at least about 60%, or at least about 70%, or at least about 80%, or at least about 90% or up to and including a 100% increase or any increase between 1 0-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level, of the proteasome nuclear localization.

As indicated above, the methods of the invention involve the step of contacting the cell/s with the agonist/s of the invention. As used herein “contacting the cell” and the like, refers to any means of introducing at least one agent described herein, specifically, the mTOR agonist/s of the invention, more specifically, at least one aromatic amino acid, any mimetics thereof, or any compound or agent that directly or indirectly increase the level, stability and/or bioavailability of the at least one aromatic amino acid residue/s, or a composition comprising at least one mTOR agonist/s described herein into a target cell in vitro, ex vivo or in vivo, including by chemical and physical means, whether directly or indirectly or whether the at least one mTOR agonist/s or the composition comprising the at least one mTOR agonist/s physically contacts the cell directly or is introduced into an environment (e.g., culture medium, body cavity, organ and/or tissue) in which the cell is present or to which the cell is added. It is to be understood that the cells contacted with the at least one agent or composition comprising the at least one agent described herein (e.g., Y. W and/or F) can also be simultaneously or subsequently contacted with another compound, such as a growth factor or other differentiation agent to stabilize and/or to differentiate the cells further. Contacting also is intended to encompass methods of exposing a cell, delivering to a cell, or ‘loading’ a cell with an mTOR agonist/s by viral or non-viral vectors, and wherein such mTOR agonist/s is bioactive upon delivery.

The method of delivery will be chosen for the particular agent and use (e.g., disorder characterized by or associated with processed involving short-term stress conditions as disclosed herein).

Parameters that affect delivery, as is known in the art, can include, inter alia, the cell type affected (e.g., epithelial cells, bone marrow lymphocytes, myocytes, neuronal cells and the like), and cellular location. In some embodiments, “contacting” includes administering the at least one mTOR agonist/s (e.g., Y. W and F and/or mimetics thereof) or a composition comprising the at least one mTOR agonist/s to an individual. In some embodiments, “contacting” refers to exposing a cell or an environment in which the cell is located to one or more of a Y, W, and F or any mimetic thereof described in the present disclosure. It should be understood that in some embodiments, the term “contacting” is not intended to include the in vivo exposure of cells to the agents or compositions disclosed herein that may occur naturally (i.e., as a result of digestion of an ordinary meal).

It should be appreciated that the cell can be contacted with any one of the at least one mTOR agonist/s of the present disclosure, specifically, at least one aromatic amino acid residue, more specifically, at least one of tyrosine, tryptophan, phenylalanine, and/or any mimetics thereof, together or separately. In one exemplary embodiment, a cell can be contacted with an oligopeptide, a peptide, or polypeptide comprising the at least one of tyrosine, tryptophan, phenylalanine, and/or any mimetics thereof, for example, a synthetic oligopeptide, peptide, or polypeptide containing only Y, W, and/or F residues.

In practicing the subject methods, any cell that expresses mTOR can be targeted for modulation of proteasome dynamics, Non-limiting examples of specific cell types in which mTOR can be modulated thereby modulating proteasome dynamics, include fibroblast, cells of skeletal tissue (bone (e.g., proliferative and hypertrophic chondrocytes) and cartilage), cells of epithelial tissues (e.g. liver, lung, breast, skin, bladder and kidney), cardiac and smooth muscle cells (e.g., cardiomyocytes), neural cells (glia and neurons), cells of the hypothalamus, hippocampal cells, endocrine cells (adrenal, pituitary, pancreatic islet alpha and beta cells), exocrine pancreatic cells (e.g., acinar cells), melanocytes, many different types of hematopoietic cells (e.g., macrophages, cells of B-cell or T-cell lineage, neutrophils, red blood cells, and their corresponding stem and progenitor cells, lymphoblasts), cells of both white adipose tissue and brown adipose tissue (e.g., adipocytes), and intestinal cells (e.g., Paneth cells, enterocytes, goblet cells). In some embodiments, the cell is a mammalian cell. In some embodiments, the cell is a human cell.

Still further, the disclosure provides a method of increasing mTOR activity thereby increasing proteasome nuclear localization in a subject comprising administering to a subject in need thereof at least one mTOR agonist/s comprising at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compound that modulates directly or indirectly at least one of the levels, stability and bioavailability of the at least one aromatic amino acid residue, any combinations or mixtures thereof, any vehicle, matrix, nano- or micro-particle thereof, or any composition or kit comprising the same.

In yet some more specific embodiments, the mTOR agonist used by the methods provided by the present disclosure, may comprise at least one aromatic amino acid residue or a combination of at least two aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In some specific embodiments, the mTOR agonist/s of the methods disclosed herein may comprise at least one of the following components. First component (a), comprises at least one tyrosine (Y) residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist may comprise in some embodiments additionally or alternatively, as a second component (b), at least one tryptophan (W) residue, any mTOR agonistic tryptophan mimetic, any salt or ester thereof, any multimeric and/or polymeric form of the tryptophan residue and/or of said mTOR agonistic tryptophan mimetic, or any combination or mixture thereof. In yet some further embodiments, the mTOR agonist of the invention may comprise additionally or alternatively, as a third component (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof. In some embodiments, any combination of YW, YF or FW, and any mimetics thereof is encompassed by the invention.

Still further, in some specific embodiments, the mTOR agonist/s used by the methods of the present disclosure may comprise a combination of the following three components: (a), at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist/s of the invention further comprises (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof. The mTOR agonist/s of the methods of the present disclosure further comprises (c), at least one phenylalanine residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof.

As discussed herein, the methods of the present disclosure as presented in connection with various aspects of the invention, involve the administration of several compounds, specifically, at least one mTOR agonist/s as disclosed herein, more specifically, at least one aromatic amino acid residues, specifically, at least one of tyrosine, tryptophan and/or phenylalanine, any mimetics thereof, any compound that modulates directly or indirectly at least one of the levels, stability and bioavailability of these aromatic amino acid residues and/or at least one UPS-modulating agent, for example, at least one proteasome inhibitor. It should be therefore understood that in some embodiments, each component is present in an acceptable form for administration to the subject; any two or all three components may be part of a single composition or a single molecule; and each component is co-administered with one another to the subject.

The term “co-administered”, as used herein means that all components utilized in the methods of this invention may be administered together as part of a single dosage form (such as a single composition of this invention comprising such components) or in two or three (if the third component is utilized) separate dosage forms. Alternatively, each component may be administered prior to, consecutively with, or following the administration of another component utilized in the methods of this invention as long as all components are administered within sufficient time of one another to achieve the desired effect (e.g., increased activation of mTOR, and the resulting increased nuclear localization of the proteasome). In such combination therapy treatment, or non-therapeutic applications each component is administered by conventional, but not necessarily the same, methods. The administration of a composition comprising two or more components utilized in the methods of this invention does not preclude the separate administration of one or more of the same components to said subject at another time during a course of treatment. In some embodiment, all components that are co-administered are all administered within less than 12 hours of each other. In some embodiment, all components that are co-administered are all administered within less than 8, 6, 4, 3, 2, 1, 0.5, or 0.25 hours of each other. In some embodiments, all components are administered simultaneously (e.g., at the same time) or consecutively (e.g., one right after the other). In some embodiments, the therapeutic methods of the invention comprise the step of administering an effective amount of the mTOR agonist of the present disclosure to a subject in need. An effective amount in accordance with the invention comprise any amount of each of the aromatic amino acid residues tyrosine, tryptophan, and phenylalanine (YWF), effective to inhibit proteasome translocation in cells of a subject in need, for example, a subject suffering from cancer. This effective amount in some embodiments may lead to reduction in tumor mass and volume. In yet some further embodiments, an effective amount provided to a subject may range between about 0.01 gr to about 10 gr per day/per kg of body weight. In more specific embodiments, about 0.01 gr, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.5, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 gr/kg/day or more, to about 1 gr per day/per kg. In some particular and non-limiting embodiments, the methods disclosed herein comprise the administration of all three aromatic amino acid residues Y, W, F, in an effective amount as disclosed herein above in connection with other aspects of the invention. More specifically, in some embodiments, the methods disclosed herein comprise the administration of the aromatic amino acids Y, W and F, in a concentration ranging between about 0.01 mM to about 30 mM or more, provided that the concentration of each of the aromatic amino acid residues is less than 45 mM, and in some further embodiments, the concentration is no more than 35 mM, as discussed in connection with other aspects of the present disclosure. In yet some further embodiment, the methods disclosed herein comprise the administration of an amount of between about 5 gr-7 gr, to about 50 gr-70 gr of each of the aromatic amino acid residues Y, W, F. In yet some further embodiments, the effective amount used and administered by the methods disclosed herein may range between about 0.1 gr per day/per kg to about 0.9 gr per day/per kg, for each of the aromatic amino acid residues Y, W, F, and in some embodiments, no more than 0.99 gr per day/per kg, for each of the aromatic amino acid residues Y,W, F.

It should be appreciated that the methods, kits and compositions of the present disclosure may be suitable for any subject that may be any multicellular organism, specifically, any vertebrate subject, and more specifically, a mammalian subject, avian subject, fish or insect. In some specific embodiments, the prognostic as well as the therapeutic, cosmetic and agricultural methods presented by the enclosed disclosure may be applicable to mammalian subjects, specifically, human subjects. By “patient” or “subject” it is meant any mammal that may be affected by the above-mentioned conditions, and to whom the treatment and prognosis methods herein described is desired, including human, bovine, equine, canine, murine and feline subjects. Specifically, the subject is a human.

As discussed herein, the inventors revealed the role of mTOR in modulating proteasome dynamics in cells. Intriguingly, inhibition of the proteasome results in its import to the nucleus, a response which is evaded by drug-resistant multiple myeloma (MM), where the proteasome is largely localized to the cytosol even under basal, non-stressed conditions. This observation can serve as a predictive tool for decision making as for the efficacy of treatment using proteasome inhibitors. Thus, a further aspect of the present disclosure relates to a prognostic method for predicting and assessing responsiveness of a subject suffering from a pathologic disorder to a treatment regimen comprising at least one ubiquitin-proteasome system (UPS)-modulating agent, for example, at least one proteasome inhibitor, and/or at least one proteolysis-targeting chimeras (PROTACs), and optionally for monitoring disease progression. More specifically, in some embodiments the methods provided herein may comprise the following steps. In a first step (a), determining proteasome subcellular localization in at least one cell of at least one biological sample of the subject or in any fraction of the cell.

The second step (b), involves classifying the subject as: (i), a responsive subject to the treatment regimen, if proteasome subcellular localization is predominantly nuclear in at least one cell of the at least one sample of the subject. Alternatively, the subject may be classified as (ii), a drug-resistant subject if proteasome subcellular localization is cytosolic in at least one cell of at least one sample of the subject.

The method of the present disclosure thereby provides prediction, assessment and monitoring the responsiveness of a mammalian subject to the treatment regimen.

As shown by the present disclosure, proteasome dynamics play a major role in different cellular processes and therefore may affect various pathological conditions. The methods of the present disclosure are based on determining the cellular localization of the proteasome.

The first step of the method of the invention involves determining the proteasome subcelular localization in at least one cell of at least one biological sample of said subject. Various methods are known in the art for determining the proteasome cellular localization, using any suitable means, and are all applicable in the present disclosure. In some embodiments, methods for determining the proteasome localization may include immunohistochemical methods and cell fractionation. More specifically, methods applicable in the present invention may include but are not limited to Immunohistochemistry, Live cell imaging of the proteasome activity probe (ABPs), Western blot of nuclear fractions (e.g., Western blot of cells for 20 and 19S subunits), Cell fractionation, Immunofluorescence microscopy and Cryo-electron tomographic imaging.

More specifically, Cell fractionation is the process used to separate cellular components while preserving individual functions of each component. Tissue is typically homogenized in a buffer solution that is isotonic to stop osmotic damage. Mechanisms for homogenization include grinding, mincing, chopping, pressure changes, osmotic shock, freeze-thawing, and ultra-sound. The samples are then kept cold to prevent enzymatic damage. Homogenous mass of cells (cell homogenate or cell suspension) is formed. It involves grinding of cells in a suitable medium in the presence of certain enzymes with correct pH, ionic composition, and temperature. A filtration step may then be applied. This step may not be necessary depending on the source of the cells. Animal tissue however is likely to yield connective tissue which must be removed. Commonly, filtration is achieved either by pouring through gauze or with a suction filter and the relevant grade ceramic filter. Purification is achieved by differential centrifugation—the sequential increase in gravitational force results in the sequential separation of organelles according to their density. In this connection, wherein the methods of the present disclosure involve the step of determining proteasome subcellular localization in a cell or in any fractions thereof, in some embodiments, such fractions of a cell may be a result of the cell fractionation process discussed herein. A cell fraction may be in some embodiments a nuclear reaction. In yet some further embodiments, a cell fraction may be a cytosolic fraction.

Western Blot as used herein, particularly when applied to cell fractions, involves separation of a substrate from other protein by means of an acryl amide gel followed by transfer of the substrate to a membrane (e.g., nitrocellulose, nylon, or PVDF). Presence of the substrate is then detected by antibodies specific to the substrate, which are in turn detected by antibody-binding reagents.

Antibody-binding reagents may be, for example, protein A or secondary antibodies. Antibody-binding reagents may be radio labeled or enzyme-linked, as described hereinafter. Detection may be by autoradiography, colorimetric reaction, or chemiluminescence. This method allows both quantization of an amount of substrate and determination of its identity by a relative position on the membrane indicative of the protein's migration distance in the acryl amide gel during electrophoresis, resulting from the size and other characteristics of the protein.

Immuno-histochemical Analysis involves detection of a substrate in situ in fixed cells by substrate-specific antibodies. The substrate specific antibodies may be enzyme-linked or linked to fluorophore. Detection is by microscopy and is either subjective or by automatic evaluation. With enzyme-linked antibodies, a calorimetric reaction may be required. It will be appreciated that immunohistochemistry is often followed by counterstaining of the cell nuclei, using, for example, Hematoxyline or Giemsa stain.

Immunofluorescence microscopy enables visualization of proteasome subunits in the cells. In some embodiments, cells are seeded on glass cover slips and fixed with 4% PFA. Following appropriate treatment, the fixed cells are incubated with relevant first and secondary antibodies, washed and mounted. The fixed cells are then visualized using a confocal microscope (such as for example Zeiss LSM 700).

Live cell imaging of the proteasome consists in tagging the proteasomal subunits of living cells with a fluorescent probe, thereby allowing in vivo detection via confocal fluorescence microscopy. For example, the proteasomal subunits may be tagged with any tag such as GFP, e.g., the β4, Rpn2, Rpn6, and Rpn13 proteasome subunits may be C-terminally fused with GFP. Most proteasome subunits fully incorporate GFP tag into their appropriate sub-complexes, thus enabling live cell imaging of the 20S core protease (CP), the 19S regulatory particle (RP), and/or holo-26S particles.

Cryo-electron tomographic imaging is a method that facilitates in situ structural biology on a proteomic scale. In a cryo-ET study, a biological sample, a cell, tissue, or organism, is flash frozen, thinned to an appropriate thickness, and then imaged using an electron microscope. The freezing process preserves the sample in a hydrated, close-to-native state. Multiple images are captured as the sample is tilted along an axis. The images are then aligned and merged using computational techniques to reconstruct a three-dimensional picture, or tomogram. This method has been successful for mapping the locations of relatively large structures such as proteasome as well as ribosomes.

As indicated above, Proteasome activity-based probes (ABPs) may also be employed for detecting proteasome localization and activity. ABPs are small molecules consisting of a proteasome inhibitor linked to a small fluorophore. Fluorescence labeling of proteasomes occurs via a nucleophilic attack of the catalytic N-terminal threonine toward the ABP, leading to a covalent, irreversible bond between the warhead of the ABP and the proteasome active site.

Importantly, unlike fluorescently tagged proteasome subunits, the ABPs only label fully assembled, active proteasome complexes. ABPs react with proteasomes in a way that corresponds to their catalytic activity and because of their fluorescent properties, they can be imaged specifically and sensitively in cell lysates after gel-electrophoresis followed by fluorescent scanning or in living cells by fluorescence microscopy. With a few exceptions, most proteasome ABPs share a similar design, may comprise the following components:

(a) a reactive group (‘warhead’), typically an epoxyketone (EK) or vinyl sulfone (VS), at the C terminus; (b) a tri- or tetrapeptide recognition element; (c) a reporter tag for detection (often a fluorophore), typically appended at the N terminus via a linker. Consequently, the probes are frequently notated in the form label-linker-recognition element-warhead (e.g., BODIPY-Ahx3-L3-VS), or label-inhibitor (e.g., BODIPY-epoxomicin).

Proteasome ABPs may be divided into two categories: ‘broad-spectrum’, which are reactive toward most proteasome subunits, and ‘subunit-selective’, which show a strong preference for a single subunit type.

It should be understood that when referring to detection of the proteasome, the invention encompasses the detection of the 26S, or of any subunit thereof, specifically, at least one of the 20S and 19S subunits, as specified above.

The second step of the methods disclosed herein involves classifying the subject as a responsive (or responder) or a non-responsive (or non-responder) subject. As used herein, subcellular localization that is predominantly nuclear, is meant that the proteasome in the examined cell is mostly, mainly and/or primarily, localized to the nucleus. Specifically, a predominant, preponderant, major and/or principle share of the cellular proteasome display nuclear localization in the cell. More specifically, more than 50% of the proteasome in the cell is localized to the nucleus, specifically, about 51% or more, about 52% or more, about 53% or more, about 54% or more, about 55% or more, 60%, 65%, 70%, 75%, 80%, 85%, 90%, 95%, or more, 97%, 98%, 99% or even 100%, of the proteasome in the cell display nuclear localization. In some embodiments, cytosolic localization of 55% or more of the cellular proteasome in at least one cell of the subject, indicates drug resistance to the treatment regimen.

In some embodiments, the subject/s diagnosed by the methods of the present disclosure may display both, nuclear and cytosolic proteasome localization in most cells of the sample. According to some embodiments, for such subjects, a nuclear localization of about 50% or less, of the proteasome in at least one cell of the sample examined, is indicative of drug resistance. Thus, as shown by the present disclosure and discussed herein, an equal distribution of the proteasome between both compartments (cytosolic and nuclear) reflects non-responsiveness or drug resistance. More specifically, in some specific embodiments of the present disclosure, cytosolic localization of about 50% or more, or even 45% or more, of the proteasome, specifically, 45%, 50%, 55%, 60%, 65%, 70%, 75%, 80%, 85%, 90%, 95%, 97%, 98%, 99% or more, 100%, is referred to herein as cytosolic, and is indicative of non-responsiveness to a treatment regimen that comprise at least one UPS modulating agent, such as proteasome inhibitor, PROTAC, and any of the disclosed modulators.

However, a nuclear distribution of about 51% or more, and more specifically, 55% or more, of the proteasome in the cell of a subject, is referred to herein as a predominantly nuclear or as a nuclear localization, and reflects responsiveness to UPS-modulating agent, for example, at least one proteasome inhibitors or any of the modulators disclosed herein after. More specifically, nuclear localization of about 55%, 60%, 65%, 70%, 75%, 80%, 85%, 90%, 95%, 97%, 98%, 99% or even 100% of the proteasome in the cell, indicates that the subject is responsive to a treatment regimen comprising at least one UPS modulating agent, such as proteasome inhibitor, PROTAC, and any of the disclosed modulators.

It should be further understood that in some embodiments, a cytosolic localization determined for between about 1%-100%, specifically about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, 55%-60%, 60%-65%, 65%-70%, 70%-75%, 75%-80%, 80%-85%, 85%-90%, 90%-95%, 95%-100%, of the cells in the sample, indicates that said subject belongs to a pre-established drug-resistant or non-responsive population of subjects. In other words, the subject is a non-responsive subject. In some particular embodiments, such drug-resistant subjects or population of subjects may be associated with relapse of the disease. In yet some further embodiments, a nuclear localization determined for between about 1%-100%, specifically about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, 55%-60%, 60%-65%, 65%-70%, 70%-75%, 75%-80%, 80%-85%, 85%-90%, 90%-95%, 95%-100%, of the cells in the sample, indicates that said subject belongs to a pre-established drug-responsive or responder population of subjects. In other words, the subject is a responsive subject. In some particular embodiments, such drug-responsive subjects or population of subjects may be associated with good prognosis. Thus, in some embodiments, if 50% or more of the cells in the sample display cytosolic distribution of the proteasome (e.g., that about 45% or more of the cellular proteasome in the cell is cytosolic), the subject is classified as a non-responder, or drug resistant. In yet some further embodiments, if 50% or more of the cells in the sample display nuclear localization (e.g., that 51% or more, and specifically, 55% or more of the cellular proteasome is nuclear), the subject is classified as a responder.

As described hereinabove, the methods of the invention refer to determining proteasome subcellular localization value based on the relative amounts of the proteasome in the cell compartments, specifically, the cytosol and the nucleus. An equivalent distribution between both compartments, reflects non-responsiveness, or drug resistance. In other words, an equal distribution (namely, 50% or more, and in some embodiments, even 45% or more) of the proteasome in the cytosol and the nucleus, indicates non-responsiveness to UPS-modulating drugs, for example, proteasome inhibitors. As such, a value of about 40% to 60%, specifically, 40%, 45%, 50%, 55%, 60% may be used as a cutoff value. In yet some further embodiments, a value of about 50% of the proteasome in the cell, may be considered as a cutoff value. It should be noted that a “cutoff value”, sometimes referred to simply as “cutoff” herein, is a value that in some embodiments of the present disclosure, meets the requirements for both high prognostic sensitivity (true positive rate) and high prognostic specificity (true negative rate). Simply put, “sensitivity” relates to the rate of identification of the responder patients (samples) as such, out of a group of samples, whereas “specificity” relates to the rate of correct identification of responder samples as such, out of a group of samples. It should be noted that cutoff values may be also provided as control sample/s or alternatively and/or additionally, as standard curve/s that display predetermined standard values for responders, non-responders, and for subjects that display responsiveness to a certain extent (level of responsiveness, e.g., low, moderate and high). More specifically, the cutoff values reflect the result of a statistical analysis of proteasome localization value/s differences in pre-established populations of responder or non-responder. Pre-established populations as used herein refer to population of patients known to be responsive to a treatment of interest (e.g., treatment comprising at least one proteasome inhibitor), or alternatively, population of patients known to be non-responsive or drug-resistant to a treatment of interest.

It should be emphasized that the nature of the invention is such that the accumulation of further patient data may improve the accuracy of the presently provided cutoff values, which are usually based on ROC (Receiver Operating Characteristic) curves generated according to the patient data using analytical software program.

It should be appreciated that “Standard” or a “predetermined standard” as used herein, denotes either a single standard value or a plurality of standards with which the proteasome subcellular nuclear or cytosolic localization value determined for the tested sample is compared. The standards may be provided, for example, in the form of discrete numeric values or in the form of a chart for different values of proteasome localization, or alternatively, in the form of a comparative curve prepared on the basis of such standards (standard curve).

Thus, in certain embodiments, the prognostic methods of the present disclosure may optionally further involve the use of a calibration curve created by detecting and quantitating proteasome subcellular localization in cells of known populations of responders and non-responders to the indicated treatment. Obtaining such a calibration curve may be indicative to provide standard values.

As noted above, in some embodiments of the present disclosure, at least one control sample may be provided and/or used by the methods discussed herein. A “control sample” as used herein, may reflect a sample of at least one subject (a subject that is known to be a non-responder, or alternatively, known to be a responder, or sample displaying known nuclear and/or cytosolic at a certain predetermined degree), and in some embodiments, a mixture at least two, at least three, at least four, at least five, at least six, at least seven, at least eight, at least nine, at least ten or more patients, specifically, 15, 20, 25, 30, 35, 40, 45, 50, 55, 60, 65, 70, 75, 80, 85, 90, 95, 100 or more patients. A control sample may alternatively, or additionally comprise known cytosolic or nuclear protein or other cellular component that display known cellular localization that can be used as a reference for cytosolic or nuclear localization.

In some embodiments, the methods of the invention may be particularly useful for monitoring disease progression. In some embodiments, monitoring disease progression by the methods of the invention may comprise at least one of, predicting and determining disease relapse, and assessing a remission interval. In such case, the method of the invention may comprise the steps of: repeating step (a) of the method of the invention to determine proteasome subcellular localization for at least one cell of at least one more temporally-separated sample of the subject. More specifically, according to some embodiments, a method allowing monitoring disease progression as defined above may comprise first in step (a), determining proteasome subcellular localization in at least one cell of at least one biological sample of the subject or in any fraction of the cell. In some embodiments, the subject is being classified in the next step (b), as (i), a responsive subject to the treatment regimen, if proteasome subcellular localization is predominantly nuclear in at least one cell of the at least one sample of the subject. Alternatively, the subject may be classified as (ii), a drug-resistant subject if proteasome subcellular localization is cytosolic. For monitoring purpose, the determination step is repeated in step (c), for at least one cell of at least one more temporally-separated sample of the subject. The next step (d), involves predicting and/or determining disease relapse in the subject, if at least one cell of the at least one temporally separated sample examined, displays loss of proteasome nuclear localization, or alternatively, maintenance of cytosolic localization. It should be understood that in some embodiments, “loss” of proteasome nuclear localization is relevant in cases where at least one previous sample of the subject displayed proteasome nuclear localization (e.g., in case the subject has been previously classified as a responder). In yet some further embodiments, disease relapse in the subject, may be also predicted and/or determined if at least one cell of the at least one temporally separated sample examined, maintains predominant proteasome cytosolic localization revealed in a previous sample examined. In some embodiments, relapse may be also predicted in cases the proteasome is distributed in the temporally separated sample equally in the nucleus and the cytosol.

The invention thus provides prognostic methods for assessing responsiveness of a subject for a specific treatment regimen, for monitoring a disease progression and for predicting relapse of the disease in a subject. It should be noted that “Prognosis”, is defined as a forecast of the future course of a disease or disorder, based on medical knowledge. This highlights the major advantage of the invention, namely, the ability to assess responsiveness or drug-resistance and thereby predict progression of the disease, based on the proteasome dynamics evaluated in a cell of the prognosed subject. The term “relapse”, as used herein, relates to the re-occurrence of a condition, disease or disorder that affected a person in the past. Specifically, the term relates to the re-occurrence of a disease being treated with proteasome inhibitor/s.

The term “response” or “responsiveness” to a certain treatment, specifically, treatment regimen that comprise at least one UPS-modulating agent, for example, at least one proteasome inhibitor, PROTAC, or any of the modulators disclosed by the present disclosure, refers to an improvement in at least one relevant clinical parameter as compared to an untreated subject diagnosed with the same pathology (e.g., the same type, stage, degree and/or classification of the pathology), or as compared to the clinical parameters of the same subject prior to treatment with the indicated medicament.

The term “non responder” or “drug resistance” to treatment with a specific medicament, specifically, treatment regimen that comprise at least one UPS-modulating agent, for example, at least one proteasome inhibitor, refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment with the specific medicament.

In some embodiments, the at least one more temporally-separated sample may be obtained after the initiation of at least one treatment regimen comprising at least one UPS-modulating agent, for example, at least one proteasome inhibitor.

It should be understood that in some particular embodiments, at least one sample may be obtained prior to initiation of the treatment. Thus, in some embodiments, at least one sample is taken before treatment and at least one sample is obtained after treatment. However, in some embodiments, the methods disclosed herein may be applied to subjects already treated by a treatment regimen comprising at least one UPS-modulating agent, for example, at least one proteasome inhibitor. Accordingly, the first and the second samples are obtained after the initiation of the treatment. Such monitoring may therefore provide a powerful therapeutic tool used for improving and personalizing the treatment regimen offered to the treated subject.

As indicated above, in accordance with some embodiments of the invention, in order to assess the patient condition, or monitor the disease progression, as well as responsiveness to a certain treatment (e.g., comprising at least one proteasome inhibitor), at least two “temporally-separated” test samples must be collected from the examined patient and compared thereafter, in order to determine if there is any change or difference in the proteasome localization values between the samples. Such change may reflect a change in the responsiveness of the subject. In practice, to detect a change having more accurate predictive value, at least two “temporally-separated” test samples and preferably more, must be collected from the patient.

The proteasome cellular localization value is determined using the method disclosed herein, applied for each sample. As detailed above, the change in localization is calculated by determining the change in cellular localization between at least two samples obtained from the same patient in different time-points or time intervals. This period of time, also referred to as “dime interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least one week, at least two weeks, at least three weeks, at least one month, at least two months, at least three months, at least four months, at least five months, at least six months, at least one year, or even more.

The number of samples collected and used for evaluation and classification of the subject either as a responder or alternatively, as a drug resistant or as a subject that may experience relapse of the disease, may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the disease progression according to the present disclosure, it is understood that the change in nuclear or cytosolic proteasome localization value, may be calculated as an average change over at least three samples taken in different time points, or the change may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more.

In yet some further embodiments, the prognostic method is applied on a subject suffering from a pathogenic disorder. In yet some further embodiments, the diagnosed subject is suffering from at least one of, at least one proliferative disorder, and/or at least one protein misfolding disorder or deposition disorder.

In some embodiments, the proliferative disorder relevant to the method of the invention may be at least one solid or non-solid cancer, or any metastasis thereof.

In some specific embodiments, a proliferative disorder may be at least one hematological malignancy, and any related condition. Still further, in some embodiments, a protein misfolding disorder or deposition disorder may be amyloidosis and any related conditions.

In some embodiments, the method of the invention may be particularly applicable for patient affected by hematological malignancies. In more specific embodiments, such hematological malignancy or cancer may be a multiple myeloma (MM) and/or any related condition.

Accordingly, the prognostic method of the invention may be used for predicting and assessing responsiveness of a subject suffering from MM, to a treatment regimen comprising at least one UPS-modulating agent, for example, at least one proteasome inhibitor, and optionally, for monitoring MM disease progression in the subject.

In some further embodiments, the methods of the invention provide a tool (either independent or complementary tool) for classification and monitoring of disease severity and staging. More specifically, a higher nuclear localization value may be associated to a mild disease, whereas a predominant cytosolic localization may reflect a more advanced disease, that may in some embodiments involve relapse.

The prognostic methods of the invention thus, provide a diagnostic and therapeutic powerful tool for screening patients to tailor an optimal personal treatment regimen for each patient, by determining responsiveness of any patient to a treatment comprising at least one UPS-modulating agents. UPS-modulating agents as used herein, are any agents or compounds that modulate protein degradation mediated by the ubiquitin-proteasome system, and include any agents that directly or indirectly inhibit, reduce, attenuate, or alternatively, induce, elevate or increase UPS-mediated protein degradation. More specifically, UPS-modulating agents are any agents used for modulation of the ubiquitin-proteasome system by affecting proteasome localization, activity, or assembly. It should be understood that any UPS-modulating agents that affect proteasome cellular localization, agents that are affected by proteasome cellular localization, and/or agents that their biological effect is mediated directly or indirectly by proteasome cellular localization, are of particular interest in the present disclosure. In more specific embodiments such agents include, but are not limited to agents which affect ubiquitin conjugation (e.g., modulators of ubiquitin ligases, E3s); agents which modulate the activity of deubiquitinating enzymes (DUBs); drugs targeting the Unfolded Protein Response (UPR); Calcineurin pathway inhibitors; and/or any agents that their activity affect directly or indirectly proteasomal degradation. Ubiquitin conjugation, as used herein, refers to a process covalently attaching ubiquitin to target substrates, an intermediate step which is essential for their proteasome-mediated recognition and subsequent degradation by the proteasome.

In some specific embodiments, UPS-modulating agents applicable in the present invention, specifically, in the prognostic methods and kits disclosed herein, include but are not limited to any drugs that do not affect directly the proteasome but affect conjugation and DUBs. In some embodiments, UPS-modulating agents may include: (i) drugs targeting the Unfolded Protein Response (UPR), for example, proteasome inhibitors; (ii) drugs that require proteasome activity as part of their mechanism of action, for example, PROTACs and IMiDs; and (iii) drugs that target the Calcineurin pathway.

Thus, in some specific embodiments, the prognostic methods of the invention provide a therapeutic tool for determining responsiveness to a treatment comprising at least one drug that targets the Unfolded Protein Response (UPR). The Unfolded Protein Response (UPR), including the Endoplasmic Reticulum-Associated Degradation (ERAD) pathway, is involved in cellular protein quality control (dysregulated in numerous diseases), inflammation, and various other processes. The UPR depends on the proteolytic activity of the proteasome. Drugs targeting this pathway, also referred to herein as modulators of the UPR (specifically, drugs that up or regulate processes or compounds that act upstream or downstream to this pathway) include, among others, proteasome inhibitors, monoclonal antibodies targeting interleukins (e.g. Ustekinumab, Secukinumab) or TNFα (e.g. Infliximab, Adalimumab). These modulations of the UPR are used for example in the treatment of inflammatory bowel disease, psoriasis, arthritis, and potentially, other inflammatory diseases (e.g. Rheumatoid Arthritis). TNFα is also implied in some types of Amyloidosis. In some specific embodiments, the prognostic methods of the invention provide a therapeutic tool for determining responsiveness to a treatment comprising at least one proteasome inhibitor.

In some embodiments, at least one proteasome inhibitor applicable in the present invention may include any one of Bortezomib, Carfilzomib, Ixazomib, Marizomib, Oprozomib and Selinexor.

Proteasome Inhibitors as used herein, are drugs that block the action of proteasomes, by affecting the activity, localization/distribution and/or stability of the proteasome, which may be employed in the treatment of cancer. Still further, a proteasome inhibitor reduces, inhibits, decreases the activity and function of the proteasome, specifically degradation of cellular and/or nuclear proteins, specifically in about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, 55%-60%, 60%-65%, 65%-70%, 70%-75%, 75%-80%, 80%-85%, 85%-90%, 90%-95%, 95%-100%, as compared to the non-inhibited activity. To date, three of them are approved for use in treating multiple myeloma, i.e., Bortezomib, Carfilzomib and Ixazomib.

Additional examples of proteasome inhibitors include but are not limited to: Marizomib (salinosporamide A), Oprozomib (ONX-0912), delanzomib (CEP-18770), Disulfiram, Epigallocatechin-3-gallate, Lactacystin, Epoxomicin, MG132 and Beta-hydroxy beta-methylbutyrate.

In some specific embodiment, the proteasome inhibitor applicable in the methods, compositions and kits of the present disclosure may be Bortezomib. Bortezomib, sold under the brand names Velcade, Chemobort, Bortecad, among others, is an anti-cancer medication used to treat multiple myeloma and mantle cell lymphoma. This includes multiple myeloma in those who have and have not previously received treatment. It is generally used together with other medications.

Bortezomid has the following chemical structure, as denoted by Formula I:

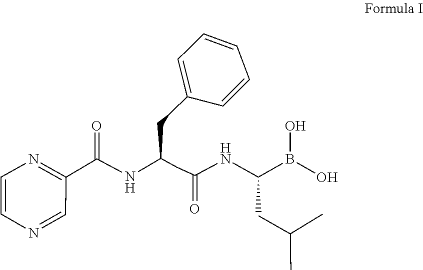

The systematic (IUPAC) name of Bortezomid is [(1R)-3-methyl-1-({(2S)-3-phenyl-2-[(pyrazin-2-ylcarbonyl)amino]propanoyl}amino)butyl]boronic acid (C₁₉H₂₅BN₄O₄; CAS number 179324-69-7). The molecular weight of the form of Bortezomid depicted above is 384.237 gram/mol.

In some specific embodiment, the proteasome inhibitor applicable in the methods, compositions and kits of the present disclosure, may be Carfilzomib. Carfllzomib (marketed under the trade name Kyprolis) is an anti-cancer drug acting as a selective proteasome inhibitor. Chemically, it is a tetrapeptide epoxyketone and an analog of epoxomicin. Carfilzomib covalently binds and inhibits the chymotrypsin-like activity of the 20S proteasome. Carfilzomib displays minimal interactions with non-proteasomal targets, thereby improving safety profiles over bortezomib.

Carfilzomib has the following chemical structure, as denoted by Formula II:

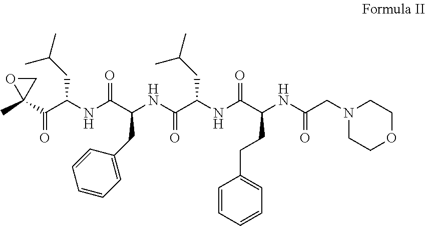 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

The systematic (IUPAC) name of Carfilzomib is (2S)-4-Methyl-N-[(2S)-1-[[(2S)-4-methyl-1-[(2R)-2-methyloxiran-2-yl]-1-oxopentan-2-yl]amino]-1-oxo-3-phenylpropan-2-yl]-2-[[(2S)-2-[(2-morpholin-4-ylacetyl)amino]-4-phenylbutanoyl]amino]pentanamide (C₄₀H₅₇N₅O₇; CAS number 868540-17-4). The molecular weight of the form of Carfilzomib depicted above is 719.91 gram/mol.

In some specific embodiment, the proteasome inhibitor applicable in the methods, compositions and kits of the present disclosure, may be Ixazomib. Ixazomib (trade name Ninlaro) is a drug for the treatment of multiple myelomain combination with other drugs. It is taken by mouth in form of capsules.

Like the older bortezomib (which can only be given by injection), it acts as a proteasome inhibitor, has orphan drug status in the US and Europe, and is a boronic acid derivative. Ixazomib is used in combination with lenalidomide and dexamethasone for the treatment of multiple myeloma in adults after at least one prior therapy.

At therapeutic concentrations, Ixazomib selectively and reversibly inhibits the protein proteasome subunit beta type-5 (PSMB5) with a dissociation half-life of 18 minutes. This mechanism is the same as of bortezomib, which has a much longer dissociation half-life of 110 minutes; the related drug carfilzomib, by contrast, blocks PSMB5 irreversibly.

Ixazomib has the following chemical structure, as denoted by Formula III:

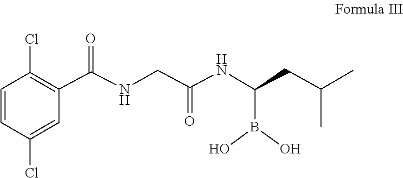 [SMILES: CC(C)C[C@H](NC(=O)CNC(=O)Cc1cc(Cl)ccc1Cl)B(O)O]

The systematic (IUPAC) name of Ixazomib is N²-(2,5-Dichlorobenzoyl)-N-[(1R)-1-(dihydroxyboryl)-3-methylbutyl]glycinamide (C₁₄H₁₉BCl₂N₂O₄; CAS number:. 1072833-77-2). The molecular weight of the form of Ixazomib depicted above is 361.03 gram/mol.

In some specific embodiment, the proteasome inhibitor applicable in the methods, compositions and kits of the present disclosure may be Marizomib. Salinosporamide A (Marizomib) is a potent proteasome inhibitor being studied as a potential anticancer agent. This marine natural product is produced by the obligate marine bacteria Salinispora tropica and Salinispora arenicola, which are found in ocean sediment. Salinosporamide A belongs to a family of compounds, known collectively as salinosporamides, which possess a densely functionalized γ-lactam-β-lactone bicyclic core. Salinosporamide A inhibits proteasome activity by covalently modifying the active site threonine residues of the 20S proteasome.

Marizomib has the following chemical structure, as denoted by Formula IV:

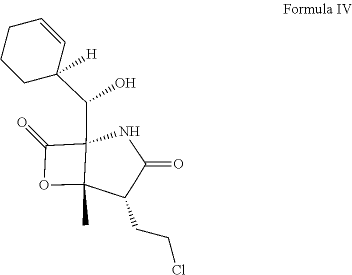

The systematic (IUPAC) name of Marizomib is (4R,5S)-4-(2-chloroethyl)-1-((1S)-cyclohex-2-enyl(hydroxy)methyl)-5-methyl-6-oxa-2-azabicyclo[3.2.0]heptane-3,7-dione (C₁₅H₂₀ClNO₄; CAS number: 437742-34-2). The molecular weight of the form of Marizomib depicted above is 313.781 gram/mol.

In some specific embodiment, the proteasome inhibitor applicable in the methods, compositions and kits of the present disclosure, may be Oprozomib. Oprozomib (codenamed ONX 0912 and PR-047) is an orally active second-generation proteasome inhibitor. It selectively inhibits chymotrypsin-like activity of both the constitutive proteasome (PSMB5) and immunoproteasome (LMP7).

It is being investigated for the treatment of hematologic malignancies, specifically, multiple myeloma. Being an epoxyketone derivative, oprozomib is structurally related to carfilzomib and has the added benefit of being orally bioavailable. Like carfilzomib, it is active against bortezomib-resistant multiple myeloma cells. Oprozomib was granted orphan drug status for the treatment of Waldenström's macroglobulinaemia and multiple myeloma. Oprozomib has the following chemical structure, as denoted by Formula V:

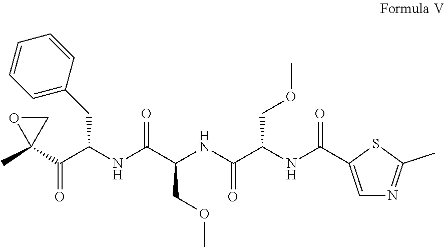

The systematic (IUPAC) name of Oprozomib is N-[(2S)-3-methoxy-1-[[(2S)-3-methoxy-1-[[(2S)-1-[(2R)-2-methyloxiran-2-yl]-1-oxo-3-phenylpropan-2-yl]amino]-1-oxopropan-2-yl]amino]-1-oxopropan-2-yl]-2-methyl-1,3-thiazole-5-carboxamide (C₂₅H₃₂N₄O₇S; CAS number: 935888-69-0). The molecular weight of the form of Oprozomib depicted above is 532.61 gram/mol.

In some specific embodiment, the proteasome inhibitor applicable in the present invention may be Selinexor. Selinexor (INN, trade name Xpovio; development code KPT-330) is a selective inhibitor of nuclear export used as an anti-cancer drug. It works by binding to exportin 1 and thus blocking the transport of several proteins involved in cancer-cell growth from the cell nucleus to the cytoplasm, which ultimately arrests the cell cycle and leads to apoptosis.

Selinexor was granted accelerated approval by the U.S. Food and Drug Administration (FDA) for use in combination with the corticosteroid dexamethasone for the treatment of adult patients with relapsed refractory multiple myeloma (RRMM) who have received at least four prior therapies and whose disease is resistant to several other forms of treatment, including at least two proteasome inhibitors, at least two immunomodulatory agents, and an anti-CD38 monoclonal antibody.

Selinexor has the following chemical structure, as denoted by Formula VI:

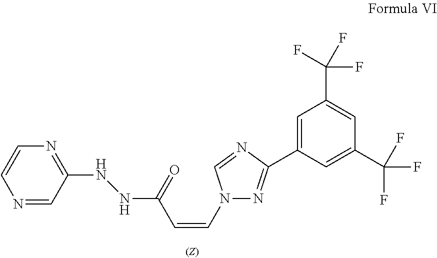 [SMILES: O=C(/C=C\n1cnc(-c2cc(C(F)(F)F)cc(C(F)(F)F)c2)n1)NNc1cnccn1]

The systematic (IUPAC) name of Selinexor is (2Z)-3-{3-[3, 5-Bis(trifluoromethyl)phenyl]-1,2,4-triazol-1-yl}-N′-pyrazin-2-ylprop-2-enehydrazide (C₁₇H₁₁F₆N₇O; CAS number:. 1393477-72-9). The molecular weight of the form of Selinexor depicted above is 443.313 gram/mol.

In yet some further embodiments, the prognostic methods of the invention provide a therapeutic tool for determining responsiveness to a treatment comprising at least one PROTAC and related molecules. More specifically, a proteolysis targeting chimera (PROTAC) is a heterobifunctional small molecule composed of two active domains and a linker capable of inducing targeted protein degradation by the ubiquitin-proteasome system. Mechanistically, this can be achieved via chemical ligands that induce molecular proximity between an E3 ubiquitin ligase and a protein of interest, leading to ubiquitination and degradation of the protein of interest. More specifically, PROTACs consist of two covalently linked protein-binding molecules: one capable of engaging an E3 ubiquitin ligase, and another that binds to a target protein meant for degradation. Recruitment of the E3 ligase to the target protein results in ubiquitination and subsequent degradation of the target protein by the proteasome. PROTACs, for example, PROTACs developed by ARVINAS LTD., applicable in the present disclosure include ARV-110 that is a potent, selective, orally available androgen receptor (AR) degrader, ARV-766 and AR-7, (that are AR Backups), ARV-471 (an oral estrogen receptor (ER)-targeting PROTAC® protein degrader for the potential treatment of patients with locally advanced or metastatic ER positive/HER2 negative breast cancer) and the like.

In yet some further embodiments, the prognostic methods of the invention provide a therapeutic tool for determining responsiveness to a treatment comprising at least one IMiD. Imunomodulatory drugs (IMiDs) are a group of compounds that are analogues of thalidomide with anti-angiogenic properties and potent anti-inflammatory effects owing to its anti-tumor necrosis factor (TNF) α activity. More specifically, Thalidomide, that is a synthetic derivative of glutamic acid, and its analogs, lenalidomide and pomalidomide are IMiDs effective in the treatment of multiple myeloma and other hematological malignancies. Recent studies showed that IMiDs bind to CRBN, a substrate receptor of CRL4 E3 ligase, to induce the ubiquitination and degradation of IKZF1 and IKZF3 in multiple myeloma cells, contributing to their anti-myeloma activity. Similarly, lenalidomide exerts therapeutic efficacy via inducing ubiquitination and degradation of CK1α in MDS with deletion of chromosome 5q. Recently, novel thalidomide analogs have been designed for better clinical efficacy, including CC-122 (avadomide), CC-220 (iberdomide) and CC-885. It should be therefore appreciated, that any of the ImiDs discussed herein may be applicable for the methods and kits of the present disclosure.

Still further, in some embodiments, the prognostic methods of the invention provide a therapeutic tool for determining responsiveness to a treatment regimen comprising at least one Calcineurin pathway modulator. More specifically, the Calcineurin pathway is a key component of the immune system and is relying on proteasomal activity for some of its key cellular and physiological effects. Calcineurin inhibitors such as Cyclosporine and Tacrolimus are widely used as immunosuppressive agents following organ transplantation, and for the treatment of several autoimmune diseases. Thus, in some embodiments, the prognostic methods of the invention provide a therapeutic tool for determining responsiveness to a treatment regimen comprising any Calcineurin pathway inhibitor.

It should be appreciated that any of the UPS-modulating agents discussed herein, specifically, any agents that affect and/or affected by proteasome cellular localization, and/or agents that their biological effect is mediated directly or indirectly by proteasome cellular localization, are applicable for any of the aspects discussed in the present disclosure.

As indicated herein, the methods of the invention involve the step of determining proteasome localization in at least one cell in a sample. Biological sample is any sample obtained from the subject that comprise at least one cell or any fraction thereof. In some specific embodiments, sample applicable in the methods of the invention may include bone marrow, lymph fluid, blood cells, blood, serum, plasma, semen, spinal fluid or CSF, the external secretions of the skin, respiratory, intestinal, and genitourinary tracts, any sample obtained from any organ or tissue, any sample obtained by lavage, optionally of the breast ductal system, or of the uterus, plural effusion, samples of in vitro or ex vivo cell culture and cell culture constituents. In some specific embodiments, the biological sample may result from a biopsy. A biopsy is a medical test commonly performed by a surgeon. The process involves extraction of sample cells or tissues from the patient. The tissue obtained is generally examined under a microscope by a pathologist for initial assessment and may also be analyzed for proteasome localization as discussed by the present disclosure. When an entire lump or suspicious area is removed, the procedure is called an excisional biopsy. An incisional biopsy or core biopsy samples a portion of the abnormal tissue without attempting to remove the entire lesion or tumor. When a sample of tissue or fluid is removed with a needle in such a way that cells are removed without preserving the histological architecture of the tissue cells, the procedure is called a needle aspiration biopsy. Still further, the sample/s may be obtained from the described tissues ectomized from a patient (e.g., in case of therapeutic ectomy).

In some specific embodiments, particularly where MM patients are prognosed and monitored, the sample examined by the methods of the invention may be a bone marrow sample.

By assessing the responsiveness of the subject to a certain optional treatment reginen comprising at least one UPS-modulating agent, for example, at least one proteasome inhibitor, PROTAC, or any of the modulaors disclosed by the present disclosure, and predicting the potential relapse of the disease in a certain patient, the present disclosure provides a tool for tailoring a specific and personal treatment regimen for the patient.

Thus, a further aspect of the invention relates to a method for determining a personalized treatment regimen for a subject suffering from a pathologic disorder. More specifically, the method of the invention may comprise the following steps:

First in step (a), determining proteasome subcellular localization in at least one cell of at least one biological sample of the subject, or in any fraction of the cell.

The next step (b), involves classifying the subject as: (i) a responsive subject to at least one treatment regimen comprising at least one UPS-modulating agent, for example, at least one UPS-modulating agent, for example, at least one proteasome inhibitor (or any of the disclosed UPS modulators), if proteasome subcellular localization is predominantly nuclear; or (ii) a drug-resistant subject, to the treatment regimen, if proteasome subcellular localization is cytosolic. In some embodiments, subjects that display in at least one cell of at least one sample, both, nuclear and cytosolic proteasome localization, are classified as drug-resistant or as non-responders, if only 50% or less of the proteasome in at least one cell of the sample displays a nuclear or predominant nuclear localization. In some embodiments, the determination step, as well as the classification steps as described in connection with other aspects of the invention, specifically, the diagnostic and prognostic methods discussed herein above, also apply for this aspect as well.

The next step (c), involves the selection of an appropriate treatment regimen. Specifically, in some embodiments, a subject classified as a responder is administered with an effective amount of at least one UPS-modulating agent, for example, at least one proteasome inhibitor, any combinations thereof or any compositions comprising the same. In some other embodiments, subjects classified as drug-resistant or as non-responders will not be treated with the at least one UPS-modulating agent, for example, at least one UPS-modulating agent, for example, at least one proteasome inhibitor, PROTACs or any of the disclosed UPS modulators.

Still further, in some embodiments, the method for determining a personalized treatment regimen in accordance with the invention may comprise the step of administering to a subject classified as a drug-resistant to a treatment regimen comprising at least one UPS-modulating agent, for example, at least one proteasome inhibitor, an effective amount of at least one selective modulator of proteasome translocation, specifically, a selective inhibitor of proteasome translocation to the cytosol, and/or mammalian target of rapamycin (mTOR) agonist, or any combinations thereof, optionally, with at least one UPS-modulating agent, specifically, at least one proteasome inhibitor and/or at least one therapeutic agent.

In some embodiments, the additional therapeutic agent may be at least one agent enhancing a short-term stress condition or process. In more specific embodiments, the additional therapeutic agent may be at least one agent that leads to, enhances, and/or aggravates hypoxia. In some specific embodiments, agents that lead to or cause hypoxia, may be agents that inhibit or reduce angiogenesis. Non-limiting examples of angiogenesis inhibitors useful in the methods, compositions and kits of the present disclosure include at least one of: VEGF inhibitors, for example, anti-VEGF antibodies such as Bevacizumab (Avastin®), and Ramucirumab (Cyramza®), VEGF fusion proteins such as Ziv-aflibercept (Zaltrap®), kinase inhibitors such as Vandetanib (Caprelsa®), Sunitinib (Sutent®), Sorafenib (Nexavar®), Regorafenib (Stivarga®), Pazopanib (Votrient®), Cabozantinib (Cometriq®), Axitinib (Inlyta®), and agents involved with degradation of proteins (e.g., via interaction with E3 ligases) such as Thalidomide (Synovir, Thalomid®), and related drugs, for example, Lenalidomide (Revlimid®).

In some embodiments, the at least one mTOR agonist/s provided by the present disclosure, may comprise at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compound that modulates directly or indirectly at least one of the levels, stability and bioavailability of the at least one aromatic amino acid residue, any combinations or mixtures thereof, any vehicle, matrix, nano- or micro-particle thereof, any composition or kit comprising the same.

In yet some more specific embodiments, the mTOR agonist used by the methods provided by the present disclosure, may comprise at least one aromatic amino acid residue or a combination of at least two aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In some specific embodiments, the mTOR agonist of the methods disclosed herein may comprise at least one of the following components. First component (a), comprises at least one tyrosine (Y) residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist may comprise in some embodiments as a second component (b), at least one tryptophan (W) residue, any mTOR agonistic tryptophan mimetic, any salt or ester thereof, any multimeric and/or polymeric form of said tryptophan residue and/or of said mTOR agonistic tryptophan mimetic, or any combination or mixture thereof. In yet some further embodiments, the mTOR agonist of the invention may comprise as a third component (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof.

Still further, in some specific embodiments, the mTOR agonist used by the methods of the present disclosure may comprise a combination of the following three components: first component (a), comprises at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist of the invention further comprises component (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof. The mTOR agonist of the methods of the present disclosure further comprises component (c), at least one phenylalanine residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof. It should be understood that the mTOR agonists that also act in some specific embodiments as selective modulators of proteasome dynamics, applicable in the present aspect are any of the mTOR agonists specified in connection with other aspects of the invention.

In yet some further embodiments, the subject may be subjected to, and/or was subjected to in the past to a treatment regimen comprising at least one UPS-modulating agent, for example, at least one proteasome inhibitor, PROTACs, or any of the disclosed modulators. In some embodiments, such subject is monitored for disease progression. According to these embodiments, the method comprising the steps of: First (a), determining proteasome subcellular localization in at least one cell (or a cell fraction) of at least one biological sample of the subject. It should be noted that at least one of the examined sample/s is obtained after the initiation of the treatment regimen.

The next step (b), involves determining at least one of: (i) a disease relapse and/or loss of responsiveness, and/or drug-resistance of the subject, if at least one cell of the sample displays loss of proteasome nuclear localization, or maintained cytosolic localization, or alternatively (ii), responsiveness or maintained responsiveness of the subject, if at least one cell of the sample displays maintained predominant proteasome nuclear localization.

The next step (c), involves selecting the appropriate treatment regimen. More specifically, ceasing a treatment regimen comprising at least one UPS-modulating agent, for example, at least one proteasome inhibitor, PROTACs, or any of the disclosed modulators, of a subject displaying disease relapse and/or loss of responsiveness, and/or drug-resistance (either maintained or newly occurring). Alternatively, this step may comprise maintaining the treatment regimen of a subject displaying responsiveness or maintained responsiveness.

It should be understood that in some embodiments, the subject (either displaying maintained responsiveness, or loss of responsiveness) has been identified or classified as a responder prior to initiation of the treatment, or at any earlier stage/s during the treatment.

In some embodiments, for subject displaying disease relapse and/or loss of responsiveness, and/or drug-resistance, the option of combining a maintained treatment with UPS-modulating agent specifically, proteasome inhibitor treatment with at least one mTOR agonist, or any other selective modulator of proteasome translocation or shuttling, specifically the mTOR agonists disclosed above, may be also considered.

In some embodiments, the subject is suffering from at least one of, at least one proliferative disorder, and at least one protein misfolding disorder or a deposition disorder.

In some embodiments, the proliferative disorder relevant to the method of the invention may be at least one solid and non-solid cancer.

In yet some further embodiments, the method for determining a treatment regimen in accordance with the invention may be applicable for subjects suffering from at least one proliferative disorder.

In some embodiments, such disorder may be at least one hematological malignancy. In yet some alternative embodiments, the method for determining a treatment regimen in accordance with the invention may applicable for a protein misfolding disorder or deposition disorder, for example, amyloidosis and/or any related conditions.

In some particular embodiments, the methods of the invention are applicable to protein misfolding disorder, also named proteopathy. Thus, the present disclosure provides prognostic methods and personalized therapeutic methods applicable for subjects suffering from any proteopathy, specifically, amyloidosis.

Proteopathy refers to a class of diseases in which certain proteins become structurally abnormal, and thereby disrupt the function of cells, tissues and organs of the body. Often the proteins fail to fold into their normal configuration; in this misfolded state, the proteins can become toxic in some way (a gain of toxic function) or they can lose their normal function. The proteopathies (also known as proteinopathies, protein conformational disorders, or protein misfolding diseases) include such diseases as Creutzfeldt-Jakob disease and other prion diseases, Alzheimer's disease, Parkinson's disease, amyloidosis, multiple system atrophy, and a wide range of other disorders. In some specific embodiments, the proteopathy or protein-misfolding disorder may be Amyloidosis. Specifically, Amyloidosis is a group of diseases in which abnormal proteins, known as amyloid fibrils, build up in tissue. Symptoms depend on the type and are often variable. They may include diarrhea, weight loss, feeling tired, enlargement of the tongue, bleeding, numbness, feeling faint with standing, swelling of the legs, or enlargement of the spleen.

There are about 30 different types of amyloidosis, each due to a specific protein misfolding. Some are genetic while others are acquired. They are grouped into localized and systemic forms. The four most common types of systemic disease are light chain (AL), inflammation (AA), dialysis (Aβ₂M), and hereditary and old age (ATTR). It should be understood that the prognostic and personalized therapeutic methods of the invention, as well as any of the therapeutic methods, compositions and kits disclosed herein after, may be applicable for any type of amyloidosis, specifically, any type discussed in the present disclosure.

Additional examples of protein misfolding diseases relevant to the methods of the present disclosure, include but are not limited to Alzheimer's disease, Cerebral β-amyloid angiopathy, Retinal ganglion cell degeneration in glaucoma, Prion diseases (multiple), Parkinson's disease and other synucleinopathies (multiple), Tauopathies (multiple) Frontotemporal lobar degeneration (FTLD), Amyotrophic lateral sclerosis (ALS), Huntington's disease and other trinucleotide repeat disorders (multiple), Familial British dementia, Familial Danish dementia, Hereditary cerebral hemorrhage with amyloidosis (Icelandic) (HCHWA-I), Alexander disease, Pelizaeus-Merzbacher disease, Seipinopathies, Familial amyloidotic neuropathy, Senile systemic amyloidosis, Serpinopathies (multiple), AL (light chain) amyloidosis (primary systemic amyloidosis), AH (heavy chain) amyloidosis, AA (secondary) amyloidosis, Type II diabetes, Aortic medial amyloidosis, ApoAI amyloidosis, ApoAII amyloidosis, ApoAIV amyloidosis, Familial amyloidosis of the Finnish type (FAF), Lysozyme amyloidosis, Fibrinogen amyloidosis, Dialysis amyloidosis, Inclusion body myositis/myopathy, Cataracts, Retinitis pigmentosa with rhodopsin mutations, Medullary thyroid carcinoma, Cardiac atrial amyloidosis, Pituitary prolactinoma, Hereditary lattice corneal dystrophy, Cutaneous lichen amyloidosis, Mallory bodies, Corneal lactoferrin amyloidosis, Pulmonary alveolar proteinosis, Odontogenic (Pindborg) tumor amyloid, Seminal vesicle amyloid, Apolipoprotein C2 amyloidosis, Apolipoprotein C3 amyloidosis, Lect2 amyloidosis, Insulin amyloidosis, Galectin-7 amyloidosis (primary localized cutaneous amyloidosis), Corneodesmosin amyloidosis, Enfuvirtide amyloidosis, Cystic fibrosis, Sickle cell disease.

In yet some further embodiments, since amyloidosis is also classified as a deposition disorder, the methods of the invention may be also applicable for any deposition disorder. Deposition disorder, as used herein is any disorder involving or characterized by deposition of insoluble extracellular protein fragments, or any other metabolite, that have been rendered resistant to digestion, thereby interfering and impairing tissue or organ function and may lead to organ failure.

Still further, in some embodiments the method for determining a personalized treatment regimen in accordance with the present disclosure may be applicable in malignancy, specifically, hematological cancer such as MM and/or related conditions. According to such embodiments, the methods of the invention may be used for prognosis, monitoring and/or for determining a personalized treatment regimen for a subject suffering from MM and/or any related conditions and metastasis thereof.

Multiple myeloma (MM), also known as plasma cell myeloma and simple myeloma, is a cancer of plasma cells, a type of white blood cell that normally produces antibodies. Often, no symptoms are noticed initially. As it progresses, bone pain, bleeding, frequent infections, and anemia may occur. Complications may include amyloidosis. The cause of multiple myeloma is unknown. Risk factors include obesity, radiation exposure, family history, and certain chemicals. Multiple myeloma may develop from monoclonal gammopathy of undetermined significance that progresses to smoldering myeloma. The abnormal plasma cells produce abnormal antibodies, which can cause kidney problems and overly thick blood. The plasma cells can also form a mass in the bone marrow or soft tissue. When only one tumor is present, it is called a plasmacytoma; more than one is called multiple myeloma. Multiple myeloma is diagnosed based on blood or urine tests finding abnormal antibodies, bone marrow biopsy finding cancerous plasma cells, and medical imaging finding bone lesions. Another common finding is high blood calcium levels. Because many organs can be affected by myeloma, the symptoms and signs vary greatly. A mnemonic sometimes used to remember some of the common symptoms of multiple myeloma is CRAB: C=calcium (elevated), R=renal failure, A=anemia, B=bone lesions. Myeloma has many other possible symptoms, including opportunistic infections (e.g., pneumonia) and weight loss. Multiple myeloma is considered treatable, but generally incurable. Monoclonal gammopathy of undetermined significance (MGUS) increases the risk of developing multiple myeloma. MGUS transforms to multiple myeloma at the rate of 1% to 2% per year, and almost all cases of multiple myeloma are preceded by MGUS.

Smoldering multiple myeloma increases the risk of developing multiple myeloma. Individuals diagnosed with this premalignant disorder develop multiple myeloma at a rate of 10% per year for the first 5 years, 3% per year for the next 5 years, and then 1% per year.

Obesity is related to multiple myeloma with each increase of body mass index by five increasing the risk by 11%. Studies have reported a familial predisposition to myeloma. Hyperphosphorylation of a number of proteins, the paratarg proteins, a tendency that is inherited in an autosomal dominant manner, appears a common mechanism in these families. This tendency is more common in African-American with myeloma and may contribute to the higher rates of myeloma in this group. Rarely, Epstein-Barr virus (EBV) is associated with multiple myeloma, particularly in individuals who have an immunodeficiency due to e.g. HIV/AIDS, organ transplantation, or a chronic inflammatory condition such as rheumatoid arthritis. EBV-positive multiple myeloma is classified by the World Health Organization as one form of the Epstein-Barr virus-associated lymphoproliferative diseases and termed Epstein-Barr virus-associated plasma cell myeloma. EBV-positive disease is more common in the plasmacytoma rather than multiple myeloma form of plasma cell cancer. Tissues involved in EBV+ disease typically show foci of EBV+ cells with the appearance of rapidly proliferating immature or poorly differentiated plasma cells. The cells express products of EBV genes such as EBER1 and EBER2. While the EBV contributes to the development and/or progression of most Epstein-Barr virus-associated lymphoproliferative diseases, its role in multiple myeloma is not known. However, people who are EBV-positive with localized plasmacytoma(s) are more likely to progress to multiple myeloma compared to people with EBV-negative plasmacytoma(s). This suggest that EBV may have a role in the progression of plasmacytomas to systemic multiple myeloma. It should be understood that the methods of the present disclosure may be applicable for any type or stage of MM as disclosed herein.

In some further embodiments, the prognostic methods, as well as the therapeutic methods disclosed herein after by the present disclosure, may be suitable for various solid tumors, specifically any tumor in any organ or tissue accessible to local administration. It should be therefore understood that any proliferative disorder disclosed herein in connection with other aspects of the invention, may be also applicable in the present aspect as well.

The inventors thus provide therapeutic methods that involve diagnostic step/s. More specifically, a further aspect of the invention relates to a method for treating, preventing, inhibiting, reducing, eliminating, protecting or delaying the onset of at least one of, at least one proliferative disorder and at least one protein misfolding disorder in a subject in need thereof. More specifically, the therapeutic methods of the invention may comprise the following steps:

First in step (a), determining proteasome subcellular localization in at least one cell of at least one biological sample of the subject, or in any fraction of the cell. In the next step (b), classifying the subject as: (i), a responsive subject to a treatment regimen comprising at least one UPS-modulating agent, for example, at least one proteasome inhibitor PROTACs, or any of the disclosed modulators, if proteasome subcellular localization is predominantly nuclear; or (ii) a drug-resistant subject if proteasome subcellular localization is cytosolic. The next step (c), involves selecting a treatment regimen based on the responsiveness, thereby treating said subject. In some embodiments, this step further comprises applying the appropriate therapeutic regimen of the subject.

In some embodiments, selecting and applying an appropriate treatment regimen in accordance with the invention may comprise the step of one of the following options:

A first option (i), comprises administering to a subject classified as a responder, an effective amount of at least one UPS-modulating agent, for example, at least one proteasome inhibitor, any combinations thereof or any compositions comprising the same.

In yet another option (ii), administering to a subject classified as a drug-resistant or non-responsive subject, an effective amount of at least one mTOR agonist, or any combinations thereof, optionally, with at least one UPS-modulating agent, for example, at least one proteasome inhibitor, PROTACs, or any of the disclosed modulators. Still further, in another option (iii), applicable where the subject is classified as a drug-resistant, the step comprises ceasing the treatment regimen that comprise at least one UPS-modulating agent, for example, at least one proteasome inhibitor, or any of the disclosed modulators, or any combinations thereof or any compositions comprising the same.

In some embodiments, the subject may be further administered with at least one additional therapeutic agent, for example, at least one agent enhancing a short-term stress condition or process. In more specific embodiments, such additional therapeutic agent may be at least one agent that leads to, enhances, and/or aggravates hypoxia. In some specific embodiments, agents that lead to or cause hypoxia, may be agents that inhibit or reduce angiogenesis. Non-limiting examples of angiogenesis inhibitors useful in the methods of the present disclosure include VEGF inhibitors, for example, anti-VEGF antibodies or VEGF fusion proteins, kinase inhibitors and agents involved with degradation of proteins. Still further, in some embodiments, the present disclosure encompasses combination with a treatment regimen that induces or enhances a short-term stress, for example using a restricted diet.

In some embodiments, at least one mTOR agonist comprises at least one aromatic amino acid residue, any mTOR agonistic mimetic thereof, any salt or ester thereof, any multimeric and/or polymeric form of the at least one aromatic amino acid residue and/or of the mTOR agonistic aromatic amino acid residue mimetic, any compound that modulates directly or indirectly at least one of the levels, stability and bioavailability of the at least one aromatic amino acid residue, any combinations or mixtures thereof, any vehicle, matrix, nano- or micro-particle thereof, any combinations or mixtures thereof, any composition or kit comprising the same. In yet some more specific embodiments, the mTOR agonist/s used by the methods provided by the present disclosure, may comprise at least one aromatic amino acid residue or a combination of at least two aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In some specific embodiments, the mTOR agonist of the methods disclosed herein may comprise at least one of the following components. First component (a), comprises at least one tyrosine (Y) residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist may comprise in some embodiments as a second component (b), at least one tryptophan (W) residue, any mTOR agonistic tryptophan mimetic, any salt or ester thereof, any multimeric and/or polymeric form of said tryptophan residue and/or of said mTOR agonistic tryptophan mimetic, or any combination or mixture thereof. In yet some further embodiments, the mTOR agonist of the invention may comprise as a third component (c), at least one phenylalanine (F) residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof. Still further, in some specific embodiments, the mTOR agonist used by the methods of the present disclosure may comprise a combination of the following three components: first component (a), comprises at least one tyrosine residue, any mTOR agonistic tyrosine mimetic, any salt or ester thereof, any multimeric and/or polymeric form of the tyrosine residue and/or of the mTOR agonistic tyrosine mimetic, and any combinations or mixtures thereof. The mTOR agonist of the invention further comprises component (b), at least one tryptophan residue, any mTOR agonistic tryptophan mimetic, any salt or ester thereof, any multimeric and/or polymeric form of the tryptophan residue and/or of said mTOR agonistic tryptophan mimetic, or any combination or mixture thereof. The mTOR agonist of the methods of the present disclosure further comprises component (c), at least one phenylalanine residue, any mTOR agonistic phenylalanine mimetic, any salt or ester thereof, any multimeric and/or polymeric form of the phenylalanine residue and/or of the mTOR agonistic phenylalanine mimetic, and any combinations or mixtures thereof.

In some embodiments, the selection of a treatment regimen based on responsiveness include administering to a subject classified as a responder an effective amount of at least one UPS-modulating agent, for example, at least one proteasome inhibitor, or any of the disclosed UPS modulators, any combinations thereof or any compositions comprising the same. In some embodiments, subjects classified as drug-resistant or as non-responders to the protease inhibitor/s, may not be treated with such UPS-modulating agent, specifically, proteasome inhibitor/s, or any of the disclosed UPS modulators. For drug-resistant subjects, treatment with any selective inhibitor of proteasome translocation, for example, the mTOR agonists disclosed herein, may be considered (either as a sole therapeutic compound or in combination with any other compounds, specifically, any UPS-modulating agent, for example, at least one proteasome inhibitors). Still further, in some embodiments, the selection of a treatment regimen, specifically for subjects classified as drug-resistant to UPS-modulating agent, may include in addition to the mTOR agonists of the invention, or any other selective inhibitor of proteasome translocation, also additional therapeutic agents or therapeutic or dietary regimens. In some embodiments, the additional therapeutic agent may be at least one agent enhancing a short-term stress condition or process. In more specific embodiments, the additional therapeutic agent may be at least one agent that leads to, enhances, and/or aggravates hypoxia. In some specific embodiments, agents that lead to or cause hypoxia, may be agents that inhibit or reduce angiogenesis. Non-limiting examples of angiogenesis inhibitors useful in the methods, compositions and kits of the present disclosure include at least one of: VEGF inhibitors, for example, anti-VEGF antibodies or VEGF fusion proteins, kinase inhibitors and agents involved with degradation of proteins. Still further, such stress inducing procedure may include the provision of starvation conditions by providing a restricted diet to the treated subject.

In yet some further embodiments, combining with the mTOR agonist/s of the invention (or any other selective inhibitor of proteasome translocation) may be also considered in cases of mild or moderate responsiveness, thereby increasing sensitivity to treatment with UPS-modulating agent, for example, treatment with proteasome inhibitors, or any of the other UPS-modulators disclosed by the invention.

In some embodiments, the invention further provides at least one UPS-modulating agent, for example, at least one proteasome inhibitor, or any combinations thereof with at least one mTOR agonist, for use in a method for treating, preventing, inhibiting, reducing, eliminating, protecting or delaying the onset of at least one of at least one proliferative disorder and at least one protein misfolding disorder in a subject in need thereof. In some embodiments such method comprises a preceding diagnostic step for assessing the responsiveness of a subject to at least one UPS-modulating agent, for example, at least one proteasome inhibitor. More specifically, the method involves determining proteasome subcellular localization in at least one cell of at least one biological sample of said subject. In the next step, the subject is classified as (i) a responsive subject to a treatment regimen comprising at least one UPS-modulating agent, for example, at least one proteasome inhibitor, if proteasome subcellular localization is predominantly nuclear; or as (ii) a non-responsive subject, if proteasome subcellular localization is cytosolic. The final step is a therapeutic step involving selecting the appropriate therapeutic regimen for the subject. Specifically, administering to a subject classified as a responder, an effective amount of at least one UPS-modulating agent, for example, at least one proteasome inhibitor, any combinations thereof or any compositions comprising the same. Subjects classified as drug-resistant or as non-responders to the UPS-modulating agent, for example, at least one protease inhibitor/s, will not be treated with such proteasome inhibitor/s (or will be treated with or in combination with at least one mTOR agonist and/or any other selective inhibitor of proteasome translocation).

Specifically, in chronic disorders such as MM or amyloidosis, the therapeutic methods disclosed herein may further monitor the patient thereby providing a personalized complete treatment plan for the patient.

Thus, in some embodiments, the subject is and/or was subjected to a treatment regimen comprising at least one UPS-modulating agent, for example, at least one proteasome inhibitor and is monitored for disease progression. Accordingly, the method may comprise the following steps, first in step (a), determining proteasome subcellular localization in at least one cell of at least one biological sample of the subject, or in any fraction of the cell. In some embodiments, at least one of the samples is obtained after the initiation of the treatment regimen. In the next step (b), determining any one of: (i) a disease relapse and/or loss of responsiveness, and/or drug-resistance, and/or maintained non-responsiveness, if at least one cell of said sample displays loss of proteasome nuclear localization, cytosolic localization and/or maintained cytosolic proteasome localization; or (ii) responsiveness or maintained responsiveness of the subject, if at least one cell of the sample displays maintained predominant proteasome nuclear localization. The next step (c), involves selecting and applying the appropriate treatment regimen. More specifically, in some embodiments, ceasing a treatment regimen comprising at least one UPS-modulating agent, for example, at least one proteasome inhibitor, of a subject displaying disease relapse and/or loss of responsiveness. Alternatively, such step may comprise maintaining the treatment regimen, of a subject displaying responsiveness or maintained responsiveness.

It should be understood that in some embodiments, the subject has been identified and determined as a responder prior to initiation of the treatment.

In some embodiments, for subjects displaying disease relapse and/or loss of responsiveness, the option of combining a maintained UPS-modulating agent, for example, at least one proteasome inhibitor treatment with at least one mTOR agonist may be also considered.

In some embodiments, the proliferative disorder relevant to the method of the invention may be at least one solid and non-solid cancer, or any metastases thereof.

In some embodiments, the therapeutic method of the invention may be applicable for a proliferative disorder, specifically, at least one hematological malignancy. Alternatively, the method of the invention may be applicable for at least one protein misfolding disorder or deposition disorders, specifically, amyloidosis and any related conditions.

In more specific embodiments, the invention provides therapeutic methods applicable for at least one hematological malignancy. In more specific embodiments, such hematological malignancy may be MM. Accordingly, such method is applicable for treating, preventing, inhibiting, reducing, eliminating, protecting or delaying the onset of MM, and/or any related conditions in a subject.

As providing prognostic and tailor-made therapeutic approaches, the present invention further encompasses the provision of any means, reagent or tool required for performing the methods disclosed herein. The reagents and materials required for performing the methods of the invention may be therefore provided as a kit.

The present discloser thus provides in a further aspect thereof, a kit comprising:

First component (a), comprises at least one means, and/or reagent for determining proteasome subcellular localization in at least one cell of at least one biological sample, or in any fraction of said cell. In some embodiments, the kit of the invention may optionally further comprise at least one of: (b), pre-determined calibration curve providing standard values of proteasome subcellular localization; (c), at least one control sample; and (d), instructions for use.

In some embodiments, the pre-determined calibration curve of the kit of the invention may provide standard values of least one of proteasome nuclear and cytosolic localization. In some embodiments, such value may be a value predetermined for responders and for drug resistant subjects.

In some further embodiments, the kits of the invention may further comprise specific reagents and components required for performing subcellular localization of the proteasome.

It should be appreciated that the components in the kit may depend on the method of detection of the proteasome subcellular localization and are not limited to any method. In some embodiments, the kit of the invention may further comprise at least one reagent for conducting Immunohistochemistry, Live cell imaging of the proteasome activity probe, Western blot of nuclear fractions (e.g., Western blot of cells for 20 and 19S subunits), Cell fractionation and Cryo-electron tomographic imaging.

In further embodiments, the kits of the invention may further comprise at least one device, instrument, means or any reagent for determining the proteasome subcellular localization.

In some embodiments, the kit of the invention may be particularly applicable for use in a prognostic method, for predicting and assessing responsiveness of a subject suffering from a pathologic disorder to a treatment regimen comprising at least one UPS-modulating agent, for example, at least one proteasome inhibitor, and optionally, for monitoring disease progression in the subject. Thus, in some embodiments, the kit of the invention is a prognostic kit. In yet some further embodiments, the kit of the invention is adapted for prognosis of, prediction and assessment of responsiveness of a subject suffering from a pathologic disorder to a treatment regimen comprising at least one UPS-modulating agent. In yet some further specific embodiments, the kit of the invention may be applicable for any of the diagnostic as well as the therapeutic methods of the invention, specifically, as described herein.

Thus, in some embodiments, the kits of the present disclosure may further comprise at least one therapeutic component or agent. Appropriate therapeutic components may include, but are not limited to at least one UPS-modulating agent, for example, at least one proteasome inhibitor and/or at least one selective inhibitor of proteasome translocation, specifically, the at least one mTOR agonist/s disclosed by the invention. In some embodiments, the mTOR agonist comprises at least one aromatic amino acid residue, any compound that modulates directly or indirectly at least one of the levels, stability and bioavailability of the at least one aromatic amino acid residue, any combinations or mixtures thereof or any vehicle, matrix, nano- or micro-particle thereof, as detailed by the present disclosure.

It yet some further embodiments, the kits of the present disclosure may further comprise at least one additional therapeutic agent. In more specific embodiments, such additional therapeutic agent/s may be at least one agent enhancing a short-term stress condition or process, for example, at least one agent that leads to, enhances, and/or aggravates hypoxia. In some specific embodiments, agents that lead to or cause hypoxia, may be agents that inhibit or reduce angiogenesis. Non-limiting examples of angiogenesis inhibitors useful in the methods, compositions and kits of the present disclosure include at least one of: VEGF inhibitors, for example, anti-VEGF antibodies or VEGF fusion proteins, kinase inhibitors and agents involved with degradation of proteins. Still further, the present disclosure may further comprise dietary compounds enabling the provision of a restricted diet to the subject. In some embodiments, the invention may further provide a computer software product for determining and/or optimizing a personalized treatment regimen for a subject suffering from a pathologic disorder. More specifically, the product comprising a computer readable medium in which program instructions are stored, which instructions, when read by a computer, cause the computer to:
a. determine (and optionally quantify) proteasome subcellular localization in at least one cell, or in a population of cells in a biological sample, or in any fraction of the cell;
b. determining the extent of a nuclear or cytosolic proteasome localization in a sample (specifically, determining if predominantly nuclear, cytosolic, or equally distributed); optionally,
c. compare with a standard value wherein the value reflects the ability of the subject to respond to at least one treatment regimen comprising at least one UPS-modulating agent, for example, at least one proteasome inhibitor.

As shown by the present disclosure, proteasome dynamics can be used as a powerful prognostic tool for personalized medicine, to provide the appropriate treatment regime for a subject. In some specific embodiments, the invention provides a tool for screening for patients that can be treated with compounds that modulate proteasome dynamics, specifically, inhibitors of proteasome translocation. Non-limiting example for such screening, is provided by Example 14. Thus, a further aspect of the present disclosure relates to a prognostic method for predicting and assessing responsiveness of a subject suffering from a proliferative disorder (e.g., cancer) to a selective inhibitor of proteasome translocation, and optionally for monitoring disease progression. In some embodiments, the method comprising the steps of: First (a), determining proteasome subcellular localization in at least one cell of at least one biological sample of the subject or in any fraction of the cell; and (b), classifying the subject as a responsive subject to the selective inhibitor of proteasome translocation, if proteasome subcellular localization is cytosolic or equally distributed in at least one cell of the at least one sample. In some embodiments, the method may comprise an additional and optional step of evaluation for confirming the effect of the selective inhibitor on the proteasome localization of the treated subject. Thus, in some embodiments, the method optionally further comprising the step of: (c), determining proteasome subcellular localization in at least one cell of a sample of a subject classified in step (b) as a responsive subject, upon exposure of the cells to the selective inhibitor. More specifically, responsiveness of the subject to the specific selective inhibitor is confirmed if proteasome subcellular localization is predominantly nuclear in at least one cell contacted with the selective inhibitor of proteasome translocation.

A specific embodiment for a selective inhibitor of proteasome is provided by the mTOR agonists disclosed by the invention, as discussed herein after in connection with other aspects of the invention.

In yet some further embodiments, the prognostic method discussed above, may be further used in some aspects of the invention for determining a personalized treatment regimen for a subject suffering from cancer. Still further aspects of the invention relate to therapeutic methods for treating cancer, that comprise the prognostic step discussed above, and treating a subject classified as a responder to a selective inhibitor of proteasome translocation (e.g., the YWF, or composition thereof), with the particular selective inhibitor.

A further aspect of the present disclosure relates to a screening method for identifying at least one selective modulator of proteasome translocation. In more specific embodiments, the method is directed at identifying inhibitors, or alternatively, enhancers of proteasome translocation to the cytosol. In more specific embodiments, the method comprising the steps of: First (a), determining proteasome subcellular localization of at least one cell contacted with a candidate compound under cellular stress conditions. In some embodiments, such stress conditions may be any short-term stress conditions, for example, starvation or hypoxia.

The next step (b), involves determining the subcellular localization of at least one control protein, in at least one cell contacted with the candidate compound under cellular stress conditions. It should be understood that steps (a) and (b), of the present methods can be performed either simultaneously, or alternatively, performed sequentially in either order. In some further embodiments, determination of the subcellular localization of the proteasome or the control protein may be performed either in the cell or in any fraction of said cell. In yet some further embodiments, the at least one control protein used by the method of the invention may be at least one exported control protein and/or imported control protein. The next step (c), involves determining that the candidate compound is: (i) a selective inhibitor of proteasome translocation, if proteasome subcellular localization as determined in (a), is predominantly nuclear and the subcellular localization of the at least one exported control protein of (b), is predominantly cytosolic or equally distributed in the at least one cell contacted with said candidate compound. Alternatively, or additionally, where an imported protein is used as the control protein (imported control protein), the candidate is determined as (ii) a selective enhancer of proteasome translocation to the cytoplasm, if proteasome subcellular localization as determined in (a), is predominantly cytosolic and the subcellular localization of the at least one imported control protein of (b), is predominantly nuclear in the at least one cell contacted with the candidate compound.

In some embodiments, the import and/or the export of the control imported and/or exported proteins may be mediated directly or indirectly by at least one nucleocytoplasmic transport component.

As indicated above, the control proteins used by the screening methods of the invention are any proteins exported or imported across the nuclear membrane though direct or indirect interaction with any component of the Nuclear Pore Complex, or any component involved with the nucleocytoplasmic transport. More specifically, nucleocytoplasmic transport is the translocation of any cargo (e.g., proteins and some RNPs) between the nucleus and the cytoplasm through the Nuclear Pore Complex (NPC). NPC is a huge protein complex that consists of around 30 different proteins collectively called nucleoporins (NUPs). The transport of cargo is usually mediated by a family of Nuclear Transport Receptors (NTRs) known as karyopherins. Karyopherins bind to their cargoes via recognition of nuclear localization signal (NLS) or nuclear export signal (NES). Best described NTRs are importin-Alpha, importin-Beta1, importin-Beta2 and chromosome—region maintenance 1 (CRM1/exportin-1). They all have an N-terminal RanGTP-binding domain, a C-terminal cargo-binding domain, and the capacity to bind components of the NPC. Importin-Alpha acts as an adaptor during nuclear import of proteins, recognizing and ligating the protein between importin-Beta and cargo proteins. Importin-Alpha can precisely recognize cargo proteins by virtue of classical NLS and it also has an importin-Beta binding (IBB) domain. Still further, certain proteins shuttle back and forth constantly between the nucleus and the cytoSOL (such as hnRNP proteins involved in pre-mRNA processing and mRNA export, transcription factors, cell cycle proteins, signal transduction proteins and transport carriers). Proteins that can transport back and forth between the nucleus and the cytoplasm are called shuttling proteins, or nucleocytoplasmic shuttling proteins, and usually contain a bidirectional signal that confers both import and export. Shuttling proteins often mediate the translocation of proteins and specific RNA across the nuclear membrane. Non limiting examples for shuttling proteins include for example nucleolin, P53, Myristoylated alanine-rich C kinase substrate (MARCKS), Survivin, nuclear factor E2-related factor2 (Nrf2), Improtin-Alpha1,TAR (RNA regulatory element) DNA-binding protein 43 (TDP-43), Nucleophosmin (NPM) (Acute Myeloid Leukemia) and Cas-interacting zinc finger protein (CIZ).

In some specific embodiments a control protein useful in the present invention may be any protein translocated across the nuclear membrane via nuclear export receptors, nuclear import receptors or any shuttling and/or adaptor proteins.

In some embodiments, an exported control protein as applicable in the present screening methods, may be any protein comprising at least one nuclear export signal (NES). Currently identified NESs sequences are basically leucine-rich. In yet some specific embodiments, leucine-rich NES has certain consensus sequence: Z-X₂₋₃-Z-X₂₋₃-Z-X-Z, as denoted by SEQ ID NO. 1, (wherein “Z” may be any one of L, I, V, F, M; and “X” can be any amino acid, indicated in the attached sequence listing as Xaa). In yet some specific and non-limiting embodiment, an exported control protein applicable in the present invention may comprise at least one NES sequence comprising the amino acid sequence LPPLERLTL, as denoted by SEQ ID NO. 2. In some specific and non-limiting embodiments, a NES sequence used in the screening methods of the present disclosure as a control exported protein, is derived from p62 protein. In some specific embodiments, the NES sequence comprises the amino acid sequences encoded by the nucleic acid sequence, as dented by SEQ ID NO. 3, or any variants and homologs thereof. In yet some further embodiments, the p62 derived NES sequence applicable in the present methods comprise the amino acid sequence as denoted by SEQ ID NO. 4. Alternatively, or additionally, where imported control proteins are used in the present screening methods, such control proteins may be any protein comprising at least one NLS.

In some embodiments, the imported control proteins applicable in the present invention may comprise an NLS characterized by at least one of the following consensus sequences: PKKKRKV (monopartite), as denoted by SEQ ID NO. 5, or any variants and homologs thereof, KRXXXXXXXXXXKKKL, wherein “X” can be any amino acid (bipartite), as denoted by SEQ ID NO. 6, or any variants and homologs thereof, or the non-classical NLS comprising the amino acid sequence PRVRY-NPYTTRP, as denoted b SEQ ID NO. 7, or any variants and homologs thereof. In yet some specific and non-limiting embodiments, a NLS sequence applicable in the present invention may by the SV40 NLS comprising the amino acid sequences encoded by the nucleic acid sequence as dented by SEQ ID NO. 8, or any variants and homologs thereof. In some embodiments, the encoded NLS sequence comprises the amino acid sequence as denoted by SEQ ID NO. 5. Thus, in some particular and no-limiting embodiments, specifically for identifying inhibitors of proteasome translocation, the control protein, specifically the exported control protein, is at least one substrate of at least one nuclear export receptor. Nuclear export receptors interact with and mediate the transport of different target cargos (either proteins or RNAs), having cytoplasmic cellular functions. In some embodiments, such receptors include nuclear export receptors Exportin1(Xpo1)/CRM1, Exportin4, Exportin5, Exportin-t (Xpo-t)/los1p, Exportin cellular apoptosis susceptibility protein (CAS)/Cse1p and Msn5p [Saccharomyces cerevisiae].

In some embodiments, at least one nuclear export receptor may be the CRM1/Exportin 1 (Chromosomal Maintenance 1). Thus, in some embodiments, the control protein used by the screening method of the present disclosure may be any natural or synthetic substrate of CRM1/Exportin 1, that comprises NES.

In some embodiments, the control protein used by the screening method of the present disclosure may be any natural substrate of CRM1/Exportin 1. Examples for substrates useful in the present invention may include for example, p65 subunit of NF-Kb and the ubiquitin ligase Anaphase Promoting Complex (APC), as used in the present disclosure, or any known substrate of CRM1/Exportin 1. To name but few, Snurportin 1 (involved in U snRNA import),HIV's Rev-1 protein, adenomatous polyposis coli tumor suppressor protein (APC), Cyclin-dependent kinase inhibitor 1B (CDKN1B), class II, major histocompatibility complex, transactivator (CIITA), 60S ribosomal export protein (NMD3), Ran-specific binding protein1 (RANBP1), NBP3, Ran, SWI/SNF-related matrix-associated actin-dependent regulator of chromatin subfamily B member 1 (SMARCB1), or p53, that contain the NES sequence, may be used as the exported control proteins, in the screening methods disclosed.

In yet some further embodiments, the control protein used by the screening method of the present disclosure may be any chimeric protein comprising the NES. Specifically, any tag or any reporter protein fused to the NES sequence may be used, for example, the NES-GFP exemplified by the present disclosure. Non-limiting examples for reporter proteins that may be fused to the NES sequences, to create the synthetic substrates used herein as a control protein, are described herein after in connection with NLS sequences applicable in imported control proteins used by the methods of the present disclosure.

In some embodiments, the selective inducer of proteasome translocation specifically modulates a biological process associated directly or indirectly with proteasome dynamics. In some embodiments, the modulator is a selective inhibitor of proteasome translocation. Such inhibitor may be suitable for use in treating, preventing, inhibiting, reducing, eliminating, protecting or delaying the onset of at least one condition or at least one pathologic disorder involved with at least one short term cellular stress condition/process in a subject.

In some embodiments, specifically for identifying compounds that enhance proteasome translocation, the control protein used by the methods of the present invention, specifically the imported control protein, is at least one substrate of at least one nuclear import receptor. Nuclear import receptors interact with and mediates the transport of proteins possessing their cellular functions in the nucleus. Examples for such receptors include but are not limited to Imp-Alpha/imp-Beta complex, Snurportin/imp-Beta complex, RIP-Alpha/imp-Beta complex, Imp7/imp-Beta complex, TRN1, Sxm1p/Kap108p [Saccharomyces cerevisiae], Mtr10p/Kap111p [Saccharomyces cerevisiae], Nmd5p/Kap119p [Saccharomyces cerevisiae], Kap14p [Saccharomyces cerevisiae], and Pdr6p/Kap122p [Saccharomyces cerevisiae]. Thus, any substrate of the nuclear import receptors disclosed herein, specifically, any protein comprising the NLS sequence, may be used as an imported control protein in the screening method of the present disclosure. In yet some further embodiments, the imported control protein used by the screening method of the present disclosure may be any chimeric protein comprising the NLS. Specifically, any tag or any reporter protein fused to the NLS sequence may be used, for example, the NLS-GFP, and the like.

Non-limiting examples for synthetic substrates that may be fused to the NLS, and/or the NES sequences described above, may include any tag or reporter protein. Non-limiting examples for such reporter proteins may include, but are not limited to Flag, HA, myc, or any fluorescent protein, for example, any one of GFP, EGFP, Emerald, Superfolder GFP, Azami Green, mWasabi, TagGFP, TurboGFP, AcGFP, ZsGreen, T-Sapphire, EBFP, EBFP2, Azurite, mTagBFP, mECFP, Cerulean, mTurquoise, CyPet, AmCyan1, Midori-Ishi Cyan, TagCFP, EYFP, Topaz, Venus, mCitrine, YPet, TagYFP, PhiYFP, ZsYellowl, mBanana, Kusabira Orange, Kusabira Orange2, mOrange, mOrange2, dTomato, dTomato-Tandem, TagRFP, TagRFP-T, DsRed, DsRed2, DsRed-Express (Ti), DsRed-Monomer, mTangerine, mRuby, mApple, mStrawberry, AsRed2, mRFPi, JRed, mCherry, HcRed1, mRaspberry, dKeima-Tandem, HcRed-Tandem, mPlum and AQ143, and the like.

As indicated herein, the present disclosure provides methods for screening for selective modulators of proteasome translocation. As used herein a “modulator” means any compound leading, causing or facilitating a qualitative or quantitative change, alteration, or modification in a molecule, a process, pathway, or phenomenon of interest. Specifically, translocation of the proteasome from nucleus to the cytosol. Without limitation, such change may be an increase, elevation, enhancement, augmentation of the translocation of the proteasome. In yet some alternative embodiments, the change may be decrease, reduction, inhibition, attenuation, of the proteasome translocation to the cytosol.

In some further aspect, the invention further provides a screening method for at least one mTOR modulating compound. Such mTOR modulator (either an agonist or antagonist) may be used as a modulator of proteasome dynamics. Preferably, in various pathological and/or physiological conditions and processes. The method of the invention comprises the step of determining proteasome subcellular localization in at least one cell contacted with at least one candidate compound or with a plurality of candidate compounds. In some embodiments, the cell contacted with the candidate under basal conditions. A candidate compound leading to predominant nuclear proteasome subcellular localization is classified as an mTOR agonist, and a candidate compound leading to predominant cytosolic proteasome localization is classified as an mTOR antagonist.

The candidate compound may be any inorganic or organic molecule, any small molecule, nucleic acid-based molecule, any aptamer, any peptide (L- as well as D-aa residues), or any combinations thereof. A compound to be tested may be referred to as a test compound or a candidate compound. Any compound may be used as a test or a candidate compound in various embodiments. In some embodiments a library of FDA approved compounds appropriate for human may be used. Compound libraries are commercially available from a number of companies including but not limited to Maybridge Chemical Co. (Trevillet, Cornwall, UK), Comgenex (Princeton, NJ), Microsource (New Milford, CT), Aldrich (Milwaukee, WI), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, CA), ChemDiv, (San Diego, CA), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, NJ), Interbio screen (Moscow, Russia), Interchim (Montlucon, France), Life Chemicals, Inc. (Orange, CT), Microchemistry Ltd. (Moscow, Russia), Otava, (Toronto, ON), PharmEx Ltd. (Moscow, Russia), Princeton Biomolecular (Monmouth Junction, NJ), Scientific Exchange (Center Ossipee, NH), Specs (Delft, Netherlands), TimTec (Newark, DE), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. Compounds isolated from natural sources, such as animals, bacteria, fungi, plant sources, and marine samples may be tested for the presence of potentially useful pharmaceutical compounds, specifically, selective modulators of proteasome translocation. It will be understood that the agents to be screened could also be derived or synthesized from chemical compositions or man-made compounds. In some embodiments a library useful in the present invention may comprise at least 10,000 compounds, at least 50,000 compounds, at least 100,000 compounds, at least 250,000 compounds, or more.

In some specific embodiments, a candidate compound screened by the screening methods of the invention may be a small molecule. A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

The preset disclosure provides specific modulators of proteasome translocation and screening methods for identifying these selective modulators, specifically, inhibitors. The present disclosure further demonstrated the therapeutic potential of such selective inhibitors (e.g., the YWF, triad), in selective killing of cancer cells. The invention therefore encompasses uses of any selective modulator, and specifically any selective inhibitors of proteasome translocation for selective induction of apoptosis and cell death of cancer cells. Thus, a further aspect of the present disclosure relates to a method for selective induction of apoptosis of cancer cells, by selective inhibition of proteasome translocation to the cytosol of these cells. In some embodiments, the method comprises contacting the cells with an effective amount of at least one selective inhibitor of proteasome translocation, or with any composition comprising said selective inhibitor.

In some embodiments, the selective inhibitor is an mTOR agonist, for example, the YWF of the present disclosure, or any composition thereof. In yet some further embodiments, the selective inhibitor may be any compound obtained by the screening method disclosed herein.

As indicated above, the therapeutic application of selective inhibition of proteasome translocation in cancer cells has been demonstrated by the present disclosure. The invention therefore further encompasses in an additional aspect thereof, a method for treating, preventing, inhibiting, reducing, eliminating, protecting or delaying the onset of a cancer in a subject, specifically, by selectively inhibiting proteasome translocation to the cytosol of cancer cells of the subject. In some embodiments, the method comprising the step of administering to the subject a therapeutically effective amount of at least one selective inhibitor of proteasome translocation, or with any composition comprising the selective inhibitor. In some embodiments, the selective inhibitor is an mTOR agonist, for example, the YWF of the present disclosure, or any composition thereof. In yet some further embodiments, the selective inhibitor may be any compound obtained by the screening method disclosed herein.

It should be understood hat the present disclosure further encompasses at least one selective inhibitor of proteasome translocation for use in a method for selective induction of apoptosis of cancer cells, by selective inhibition of proteasome translocation to the cytosol of these cells. Still further, the present disclosure further provides at least one selective inhibitor of proteasome translocation for use in a method for treating, preventing, inhibiting, reducing, eliminating, protecting or delaying the onset of a cancer in a subject, as discussed above.

It should be understood that any of the disorders disclosed by the present disclosure, specifically any of the cancerous disorders discussed herein before in connection with other aspects of the invention, are also applicable in the present aspects as well.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Experimental Procedures

Immunofluorescence Microscopy

Cells were seeded on glass cover slips for 36 h. Following the indicated treatments, they were fixed with 4% PFA for 15 min, washed with (phosphate-buffered saline) PBS and incubated in PBS containing 10% goat serum for 1h at room temperature, followed by 2 h incubation with the indicated primary antibody. Following extensive wash with PBS, the fixed cells were incubated with the relevant secondary antibody for 1 h, washed and mounted. Images were acquired using Zeiss LSM 700 confocal microscope (Zeiss, Oberkochen, Germany).

Cell Transfection and Protein Overexpression

CalFectin™ (SignaGen) transfection reagent was used to transfect cDNAs. Lipofectamine™ RNAiMAX (Invitrogen) was used to transfect siRNA oligonucleotides. Transfections were carried out according to the manufacturers' instructions. Cells were infected with Lentiviral vectors that when indicated, harbored a tetracycline-inducible promoter. Doxycycline (200 ng/ml) was added to induce gene expression.

Microscopic Visualization of Proteasome Subunits

The proteasome subunits α6, β1, and PSMD1 were visualized via indirect immunofluorescence, using primary and secondary antibodies listed under Key Resources Table. To observe the proteasome in live cells, we expressed the cDNAs of the β4, Rpn2, Rpn6, and Rpn13 proteasome subunits C-terminally fused with GFP. Photoconversion of proteasome-fused Dendra2 was carried out as previously described (McKinney et al., 2009 Nat Methods. 131-133).

Cell Fractionation

Cells were incubated for 20 min in fractionation buffer [20 mM HEPES pH 7.3, 10 mM KCl, 5 mM ATP, 5 mM MgCl₂, and protease inhibitor cocktail (Roche)], followed by the addition of NP-40 (to 0.1%). They were then mixed thoroughly and centrifuged at 1,000×g for 5 min. The supernatant was collected as cytosolic fraction, and the pellet (nuclei) was washed twice with PBS. To dissolve the nuclear pellet, fractionation buffer supplemented with 0.5% sodium deoxycholate was added followed by sonication.

Cell Lysates and Western Blotting

Cells were washed twice with ice cold PBS and scraped into lysis buffer (50 mM Tris-HCl, pH 7.4, 130 mM NaCl, 0.5% NP-40) supplemented with freshly added protease inhibitor cocktail, 5 mM ATP, 10 mM iodoacetamide, and 5 mM N-ethyl maleimide. Protein concentration was measured by the BCA assay according to the manufacturer's instructions (Pierce, Rockford, IL). 30 μg of cellular protein were resolved via SDS-PAGE, transferred to a nitrocellulose membrane and immunoblotted with the appropriate antibody.

Autophagic Flux Measurement

Autophagy analysis was carried out as previously described (Nyfeler et al., 2011, Mol Cell Biol (14):2867-76). Briefly, cells stably expressing RFP-GFP-LC3 were imaged using a high-throughput microscope (IXM-C, Molecular Devices). A mask representing the autophagic puncta was created based on the RFP channel, and was then used for quantification of the intensity in the GFP channel. These values were used in turn to calculate the autophagic flux. Data are presented in comparison to cells grown in complete medium.

Measurement of Degradation Rates

Cells were labeled with [³S] methionine and cysteine (20 μCi/ml) for 16 h. This was followed extensive washing and further incubation in a medium containing 2 mM of the two unlabeled amino acids for 8 h. Degradation rates were assessed by determining the release of labeled amino acids to the incubation medium relative to the radioactivity remained in the cellular proteins (using Trichloroacetic acid precipitation to separate between the two) (Gropper et al., 1991, JPEN J Parenter Enteral Nutr 15, 48-53.).

Fluorescence-Based Proteasome Activity Assays

Live cell proteasome activity was followed as previously described (Berkers et al., 2007, Mol Pharm; 4(5):739-48.). In brief, Me4BodipyFL-Ahx3Leu3VS was added to the medium to a final concentration of 1 μM. Following incubation for 15 min, the cells were visualized by a Zeiss LSM 700 confocal microscope. In vitro proteasome activity assay was carried out as previously described (Braten et al., 2016). In brief, cellular fractions were incubated at 37° C. for 30 min with 5 μM Suc-LLVY-AMC (Succinyl-Lu-Leu-Val-Tyr-amido-4-methylcoumarin) in a reaction buffer (40 mM Tris-HCl pH 7.2, 2 mM DTT, 5 mM MgCl2, 10 mM creatine phosphate, 0.1 mg/ml creatine phosphate kinase, 5 mM ATP). Reactions were stopped by adding 1% SDS, and fluorescence was measured at 360 nm/460 nm (ex/em).

Sample Preparation for Protein Mass Spectrometry

2-3 mg of cell extract protein in 8 M Urea and 100 mM ammonium bicarbonate, were incubated with DTT (2.8 mM; 30 min at 60° C.), modified with iodoacetamide (8.8 mM; 30 min at room temperature in the dark), and digested (overnight at 37° C.) with modified trypsin (Promega; 1:50 enzyme-to-substrate ratio) in 2 M Urea and 25 mM ammonium bicarbonate. Additional second trypsinization was carried out for 4 hours. The tryptic peptides were desalted using Sep-Pak C18 (Waters) and dried. 10 μg of protein were used for proteome analysis as described under Mass spectrometry.

Proteins Mass Spectrometry

Tryptic peptides were analyzed by LC-MS/MS using a Q Exactive plus mass spectrometer (Thermo Fisher Scientific) fitted with a capillary HPLC (easy nLC 1000, Thermo). The peptides were loaded onto a C18 trap column (0.3×5 mm, LC-Packings) connected on-line to a home-made capillary column (20 cm, internal diameter 75 microns) packed with Reprosil C18-Aqua (Dr. Maisch GmbH, Germany) in solvent A (0.1% formic acid in water). The peptides mixture was resolved with a 5-28% linear gradient of solvent B (95% acetonitrile with 0.1% formic acid) in water for 180 min followed by a 5 min gradient of 28-95% and 25 min at 95% acetonitrile with 0.1% formic acid at a flow rate of 0.15 μg/min. Mass spectrometry was performed in a positive mode (m/z 350-1800, resolution 70,000) using repetitively full MS scan followed by collision-induced dissociation (HCD at 35 normalized collision energy) of the 10 most dominant ions (>1 charges) selected from the first MS scan. A dynamic exclusion list was enabled with exclusion duration of 20 sec.

Proteomics Data Analysis

The mass spectrometry raw data were analyzed by the MaxQuant software (version 1.4.1.2, http://www.maxquant.org) for peak picking and quantification. This was followed by identification of the proteins using the Andromeda engine, searching against the human UniProt database with mass tolerance of 20 ppm for the precursor masses and for the fragment ions. Met oxidation, N-terminal acetylation, N-ethylmaleimide and carbamidomethyl on Cys, GlyGly on Lys, and phosphorylation on Ser, Thr and Tyr residues, were set as variable post-translational modifications. Minimal peptide length was set to six amino acids and a maximum of two mis-cleavages was allowed. Peptide and protein levels false discovery rates (FDRs) were filtered to 1% using the target-decoy strategy. Protein tables were filtered to eliminate identifications from the reverse database and from common contaminants. The MaxQuant software was used for label-free semi-quantitative analysis [based on extracted ion currents (XICs) of peptides], enabling quantification from each LC/MS run for each peptide identified in any of the experiments. In samples that were SILAC-labeled, quantification was also carried out using the MaxQuant software. Data merging and statistical tests were done by the Perseus 1.4 software.

Amino Acids Level Measurement

Metabolic analysis was carried out as previously described (MacKay et al., 2015). Briefly, cells were rapidly washed 3 times with ice-cold PBS and extracted with an aqueous solution of 50% Methanol, and 30% Acetonitrile. Samples were centrifuged at 16,000×g for 10 min at 4° C., and the supernatants were analyzed using HPLC-MS (Q-Exactive Orbitrap Mass Spectrometer (Thermo Scientific) coupled to Thermo Scientific UltiMate 3000 HPLC system). The HPLC setup consisted of a ZIC-pHILIC column (SeQuant, 150×2.1 mm, 5 μm, Merck) with a ZIC-pHILIC guard column (SeQuant, 20×2.1 mm). The aqueous mobile phase solvent was 20 mM ammonium carbonate adjusted to pH 9.4 with 0.1% ammonium hydroxide. The organic mobile phase was acetonitrile. Amino acids and other metabolites were separated over a 15 min linear gradient from 80% organic to 80% aqueous. The column temperature was 45° C., the flow rate 200 μl/min, and the run time 27 min. All metabolites were detected across a mass range of 75-1,000 m/z using the Q-Exactive mass spectrometer at a resolution of 35,000 (at 200 m/z) with electrospray ionization and polarity switching mode. Mass accuracy obtained for all metabolites was below 5 ppm. Data were acquired with Thermo Xcalibur software. The peak areas of different Amino Acids were determined using Thermo TraceFinder software through which metabolites were identified by the exact mass of the singly charged ion and by known retention time on the HPLC column. Commercially available standard compounds had been analyzed before to determine ion masses and retention times on the ZIC-pHILIC column. Protein quantitation based on the Lowry method was performed for normalization.

Cell Survival Assays

Cells were seeded in a 96-well plate at a density of 15,000 cells/well. ˜36 h later, cells were treated as described, and were visualized live, using high-throughput fluorescence microscopy (IXM-C, Molecular devices), under control environment (21% O₂, 5% CO₂, 37° C.). Hoechst 33342 was used to stain all cells, and either propidium iodide or SYTOX™ (Thermo) was used to stain dead cells.

Data analysis was performed using the Live/Dead module of the MetaXpress software (Molecular Devices).

Rat Heart Imaging

Animals were sacrificed (IP urethane 1.6 mg/kg), and the hearts transferred to a custom-built chamber and perfused using a Langendorff apparatus with oxygenized Tyrod's solutions, subjected to modifications (e.g. amino acid starvation and supplementation as indicated). Hearts were then sliced into ˜0.4 mm thick samples, which were incubated with Me₄BodipyFL-Ahx₃Leu₃VS and imaged as described above (see Fluorescent-based proteasome activity assays).

Rat Neural Culture Imaging

Animals and tissue were processed and cells seeded as previously described [Hakim et al., The effects of proteasomal inhibition on synaptic proteostasis. EMBO J. 9, e201593594 (2016)]. Following two weeks in culture, cells were treated as indicated, following by incubation with Me₄BodipyFL-Ahx₃Leu₃VS and imaging as described above (see Fluorescent-based proteasome activity assays).

Drosophila Gut Imaging

WT flies were maintained on either a yeast-cornmeal-molasses-malt extract medium (Cont.) or 5% sucrose solution (St.) for 6 hrs. Dissection, fixation and staining of intestines were carried out as described previously [Shaw et al., The Hippo pathway regulates intestinal stem cell proliferation during Drosophila adult midgut regeneration. Development 137, 4147-4158 (2010)].

Tumorigenicity

MDAMB231 (ATCC® HTB26™) or RT4 (ATCC® HTB2™) cells were dissociated with trypsin, washed with PBS, and brought to a concentration of 70×10₆ cells/ml. Cell suspension (7×10₆/0.1 ml) was inoculated subcutaneously at both flanks of 12 weeks old NOD.Cg-Prkdc_{scid}I12rg_{tm1Wj1}/SzJ (NSG) mice, JAX stock #005557 (n=8/group). After inoculated cells formed a palpable mass, 500 μl of either saline, saline supplemented with 25 mM/each of YWF, or saline supplemented with 25 mM/each of QLR, was injected subcutaneously 3 times a week at both flanks (adjacent to the growing tumor). After the largest tumor in each experiment has reached a size which could not be allowed to grow further, from an ethical point of view, all mice were sacrificed and xenografts were resected, weighed, and fixed in formalin. Paraffin-embedded sections were stained using standard immunohistochemistry protocol as described previously [Kravtsova-Ivantsiv et al., KPC1-mediated ubiquitination and proteasomal processing of NF-κB1 p105 to p50 restricts tumor growth. Cell 161, 333-347 (2015)]. Apoptotic cells were detected using Terminal deoxynucleotidyl transferase dUTP nick end labeling (TUNEL) according to the manufacturer's protocol, and via immunofluorescence against the apoptotic marker cleaved-Caspase3. Volumetric monitoring of tumors was carried out using a caliper twice a week. All animal experiments were carried out under the supervision of the accredited Animal Care Committee of the Technion.

Multiple Myeloma Bone Marrow Analysis

Thirty-three bone marrow biopsies taken from patients suspected and later confirmed for having MM, were obtained and processed following the approval of the Helsinki Committee in the RAMBAM Health Care Campus. For each such biopsy, data existed as for the efficacy of treatment with a proteasome inhibitor i.e. either responsive or refractory. Twenty-four of the biopsies were from patients diagnosed for the first time, and in eight of these patients the disease relapsed and a 2^{nd} biopsy was taken. These clinical data were kept separately from the biopsies—which were coded and stained for both a proteasome subunit and a marker for MM cells. A pathologist assessed each sample—blind to whether the patients were responsive or resistant to treatment with proteasome inhibitors—and determined the proteasome distribution in the MM cells—either predominantly cytosolic, evenly distributed, or predominantly nuclear. Five biopsies were excluded due to lack of staining for either the proteasome, MM marker, or both. Once the remaining twenty-eight coded cases were categorized histopathologically, the clinical outcome of each case was revealed, and each distribution category (nuclear preference, equal distribution, or cytosolic preference) was plotted against the clinical response—sensitive or resistant to the drug.

### Example 1

Amino Acid Starvation Induces Active Translocation of the 26S Proteasome from the Nucleus to the Cytosol

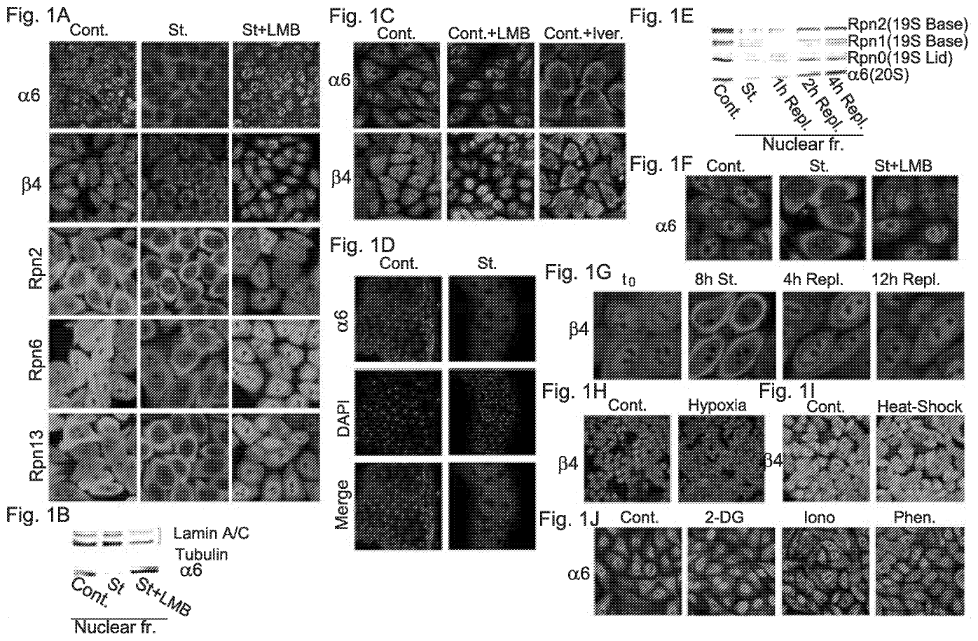

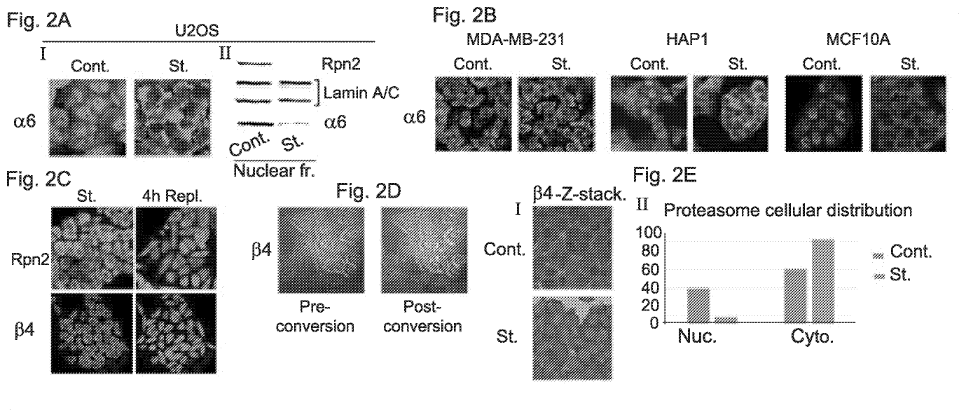

The inventors have shown previously that the proteasome undergoes autophagic degradation following amino acid starvation for longer than 24 h [1]. To shed light on the fate of the proteasome following a shorter period of stress, the localization of both the 20S and 19S complexes was monitored using fluorescent microscopy and subcellular fractionation (FIG. 1). Following amino acid starvation for 8 h, the nuclear proteasome—which constitutes a large fraction of the cellular enzyme—is translocated to the cytosol (FIGS. 1A and 1B). Treating starved cells with the exportin1 inhibitor Leptomycin B (LMB) (Kudo, N., et al (1998) Cell Res. 242, 540-547), resulted in inhibition of the translocation, showing that the recruitment is active (FIGS. 1A and 1B). LMB treatment also results in nuclear accumulation of the proteasome in non-stressed cells, demonstrating the dynamic nature of basal proteasome distribution, supported also by its cytosolic accumulation in the presence of the nuclear import inhibitor Ivermectin (Wagstaff, K. M., et al (2011) J. Biomol. Screen. 16, 192-200) (FIG. 1C). The stress-induced translocation is not unique to a single cell type, and is observed in other malignant and non-malignant cell lines (FIGS. 2A and 2B).

This observation was also tested in vivo, by visualizing the proteasome in the gut of fruit flies that were starved. Localization of the proteasome in control flies was clearly nuclear, whereas in flies fed solely on water and sugar, it was translocated to the cytosol (FIG. 1D).

### Example 2

Proteasome Recruitment is Reversible and Specific, Yet is not Limited to Nutritional Stress

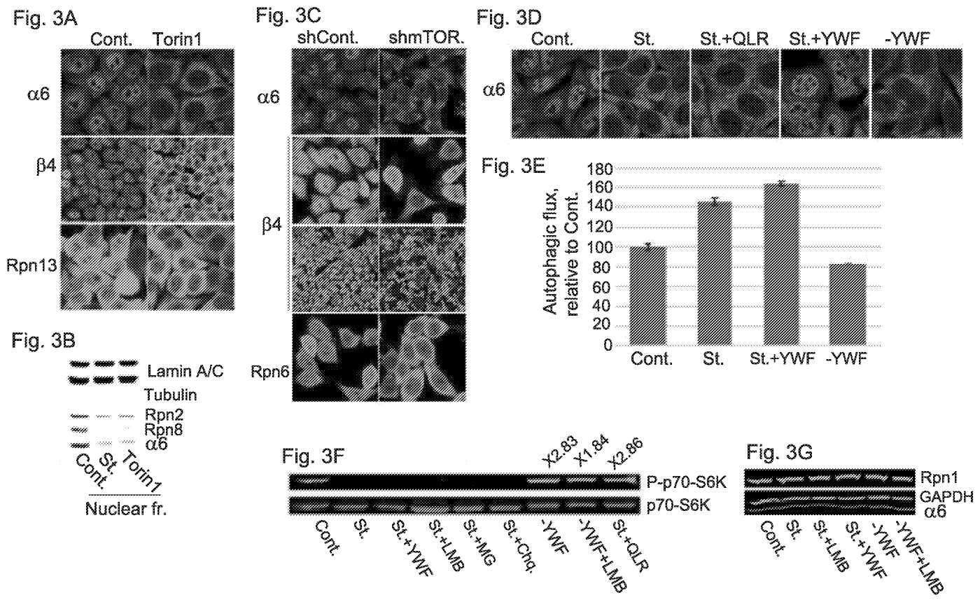

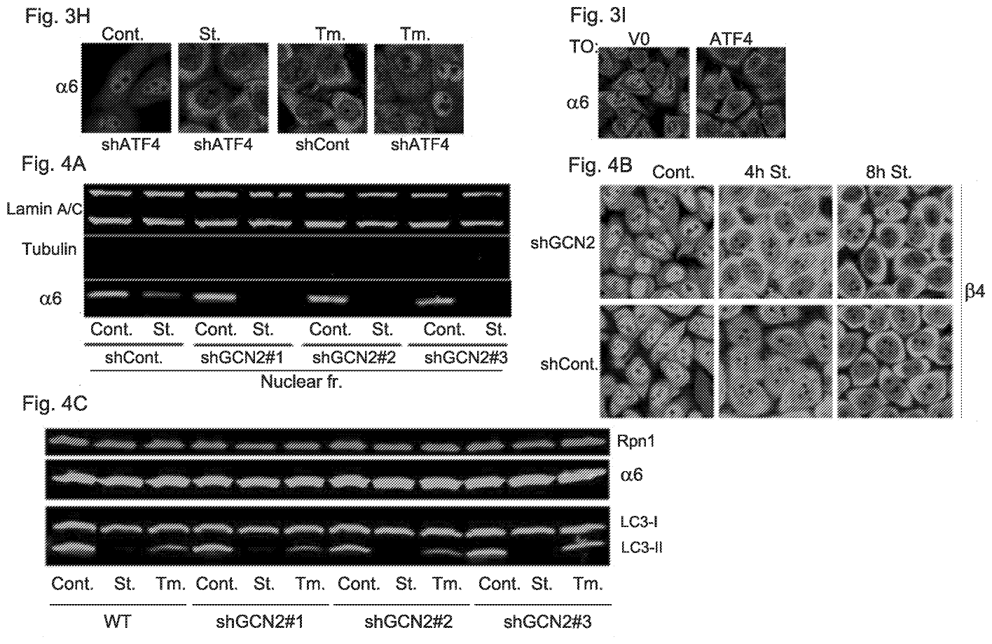

Next, the reversibility of proteasomal redistribution was tested, especially in light of the previous finding that long starvation results in autophagic degradation of the proteasome [1]. Replenishment of amino acids to 8 hours-starving cells, restores the nuclear proteasomal pool almost completely within 2-4 h (FIGS. 1E and 2C). To demonstrate that the restored proteasome does not originate from de novo synthesis, but rather from the pool of the complex that migrated previously to the cytosol, we used two independent experimental approaches: (1) amino acids were replenished in the presence of cycloheximide (CHX)—a protein synthesis inhibitor. The inventors found that it does not prevent the reappearance of the proteasome in the nucleus (FIG. 1F). (2) the proteasome was tagged with a photo-convertible fluorophore, allowing to convert pre-existing proteasomes from green to red (FIG. 2D), thereby following only complexes synthesized prior to amino acid deprivation. Live imaging of the same field of view demonstrated that amino acid starvation induces redistribution of the proteasome to the cytosol, while their replenishment results in re-localization of the previously migrated complexes back to the nucleus (FIG. 1G). The reversibility of the proteasome translocation suggests that it is not only on transit to its autophagic destruction but might serve also to stimulate proteolysis in this compartment (see below under FIG. 3). Supporting this notion is the finding that the nuclear proteasome pool comprises a significant fraction of the total cellular enzyme, reported in yeast to be as high as 80% [21]. The inventors found that in mammalian cells where the nucleus comprises only ˜ 1/10th of the cellular volume, the concentration of the nuclear proteasome is nearly ˜6 times higher than in the cytosol (FIG. 2E).

Under stress, almost the entire pool is translocated to the cytosol within a short period, providing this compartment with a considerable catalytic capacity.

To test whether proteasome recruitment in response to starvation is specific, its localization was assessed under other stresses. It was found that while hypoxia also induces proteasomal translocation (FIG. 1H), neither heat shock (FIG. 1I) nor inducers of autophagy via AMPK (FIG. 1J) result in proteasome export from the nucleus. This further distinguishes the newly identified amino acids starvation-induced translocation in mammalian cells, from the formation of proteasome storage granules following glucose starvation in yeast which is mediated via AMPK [S. J. Russell et al., J. Biol. Chem. 274, 21943-21952 (1999)]. Taken together, it appears that the shuttling of the proteasome from the nucleus to the cytosol is specific and most probably serves a pathophysiological role.

### Example 3

An as Yet Unidentified mTOR Signaling Pathway Regulates Stress-Induced Proteasome Dynamics

Since amino acids sensing is largely mediated by the mTOR signaling network [22-23], Torin1—an mTOR-specific inhibitor—was used to test whether this pathway is also responsible for starvation-induced proteasome translocation. Similar to amino acid starvation, Torin1 induces nuclear export of both 20S and 19S sub-complexes in the presence of complete growth medium (FIGS. 3A and 3B). Similarly, short hairpin RNA (shRNA) which silences mTOR expression, also lead to proteasome translocation to the cytosol (FIG. 3C). Taken together, these different experimental approaches establish the role of mTOR signaling in proteasome dynamics.

Though constituting a major signaling mediator, mTOR is not the only sensor of cellular amino acid pool. Other pathways include PIK3CA and GCN2 (Dever, T. E., et al (1992) Cell 68, 585-596; Tato, I., et al (2011) J. Biol. Chem. 286, 6128-6142; Wolfner, M., et al (1975) J. Mol. Biol. 96, 273-290). It was therefore tested whether these reported pathways are required for proteasome export following amino acid starvation. Silencing GCN2, a sensor for uncharged tRNAs and a kinase of eIF2α (Wek, S. A., et al (1995) Mol. Cell. Biol. 15, 4497-4506), does not impair proteasome export. On the contrary, it augments it (FIGS. 4A and 4B). Such a finding is in agreement with the suggested role that GCN2 plays in lowering the demand for amino acids during shortage by inhibiting protein synthesis (Suraweera, A., et al (2012) Mol. Cell 48, 242-253). Interestingly, GCN2 silencing has no effect on stimulation of autophagy (FIG. 4C), demonstrating that the UPS is responsible for most of amino acid supplementation during short-term deprivation. A different study suggested the involvement of PIK3CA and AKT in amino acid sensing (Tato, I., et al (2011) J. Biol. Chem. 286, 6128-6142). Silencing of either of these genes has no effect on proteasome translocation in response to amino acid deprivation (FIGS. 4D and 4E). Taken together, these findings leave the mTOR pathway as the sole known pathway that mediates stress-induced proteasome dynamics.

Next, it was important to identify the amino acids involved in sensing the shortage stress. The ‘canonical’ trio known to modulate mTOR activity are Gln, Leu, and Arg (QLR) (González, A., et al (2017) EMBO J. 36, 397-408; Wolfson, R. L., et al (2017) Cell Metab. 26, 301-309). These three amino acids were shown to regulate several mTOR downstream pathways, among them TFEB and ULKi-mediated autophagy (Jung, C. H., et al (2009) Mol. Biol. Cell 20, 1992-2003; Settembre, C., et al (2011) Science (80). 332, 1429-1433; Tan, H. W. S., et al (2017) Nat Commun 8, 338), and translation via phosphorylation of p70-S6K and 4EBP (von Manteuffel, S. R., et al (1996) Proc. Natl. Acad. Sci. U.S.A 93, 4076-4080; Price, D. J., et al (1992) Science 257, 973-977). It was found that unlike their effect on autophagy and translation, the addition of Gln, Leu, and Arg to the starvation medium does not prevent mTOR-mediated proteasome export (FIG. 3D).

The entire repertoire of amino acids was therefore screened in a search for one or several that affect mTOR-regulated proteasome dynamics. Tyr, Trp, and Phe (YWF)— the three aromatic amino acids—were identified as strong inhibitors of starvation-induced proteasome translocation (FIG. 3D and FIG. 4F). The addition of each of them alone to the starvation medium (in the absence of any other amino acid) has a significant effect, but combination of all three has the strongest one. In a complementary experiment, it was tested whether subtracting only YWF from the complete medium (leaving the remaining seventeen, including QLR) is sufficient to induce proteasome translocation. As can be seen in FIG. 3D, the absence of YWF is sufficient to induce proteasome recruitment to the cytosol. Importantly, the effect of YWF on proteasome movement is specific: as can be seen in FIGS. 4G and 4H, while LMB inhibits nuclear export of the p65 subunit of NF—KB and the ubiquitin ligase Anaphase Promoting Complex (APC), YWF has no effect on these two known substrates of exportin-1. Similarly, while LMB leads to nuclear accumulation of the model protein GFP fused to nuclear export signal (NES), YWF has no effect on its cellular distribution, which, as expected, is largely cytosolic (FIG. 4I). Taken together, these findings clearly show that YWF inhibits proteasome export via interference with the mTOR signaling pathway and not with the physical machinery of the nuclear export, and therefore may constitute a novel regulatory signal for this pathway.

### Example 4

Regulation of Proteasome Dynamics by YWF is Independent from mTOR-Mediated Regulation of Autophagy by QLR

At that stage, it was important to test whether YWF—the newly identified amino acids that regulate proteasome dynamics—affect mTOR downstream effects known to be governed by QLR. YWF effect is specific also as far as the signal they elicit through mTOR. While their addition to the starvation medium inhibits proteasome translocation (FIG. 3D), it does not inhibit autophagic activation (FIG. 3E). Rather, it stimulates it even further, probably since

the coping mechanism of proteasome recruitment is inhibited. In agreement with this finding, while the absence of YWF is sufficient to induce proteasome recruitment (FIG. 3D), it does not upregulate autophagy. As a matter of fact, it even downregulates it (FIG. 3E), probably due to the proteolytic activity of the recruited proteasomes. Further, it was also found that unlike QLR, YWF could not reverse the effect of starvation on mTOR-mediated p70-S6K phosphorylation (FIG. 3F). Also, subtraction of YWF does not inhibit p70-S6K phosphorylation, and even stimulates it (FIG. 3F). This effect is probably due to supplementation of amino acids, mediated via proteasome-stimulated degradation of cytosolic proteins (see below). The addition of either proteasome or autophagy inhibitors to starvation media does not rescue phosphorylation (FIG. 3F). Taken together, these findings strongly suggest that YWF exert their signaling effect at the mTOR level, and not downstream through a direct effect on the proteasome or the autophagic machinery. Of note is that under the tested conditions, both sub-complexes of the 26S proteasome have remained stable (FIG. 3G), underscoring the previous report of the present inventors that the complex is stable during short-term stress [1] and all changes reported in the present disclosure are due to its redistribution.

Thus, while by using different inhibitors it was shown that mTOR relays differential downstream signals [C. C. Thoreen et al., J. Biol. Chem. 284, 8023-8032 (2009)], the inventors show that different sets of agonistic amino acids lead to different downstream effects.

### Example 5

Proteasome Shuttling Following Unfolded Protein Stress is Mediated by ATF4, and is Regulated Distinctively from the Signaling Pathway of Starvation

As described above, agents such as Tunicamycin, which stimulate the UPR via the eIF2α-ATF4 signaling pathway, are also inducing proteasome nuclear export (FIG. 1D and FIG. 2C). Stimulation of the eIF2α-ATF4 pathway is mediated through phosphorylation of eIF2α by several protein kinases, each activated by a different stimulus. In the case of UPR, the kinase PERK phosphorylates eIF2α (Harding, H. P., Zhang, Y., and Ron, D. (1999) Nature 397, 271-274), while amino acid starvation upregulates this pathway through stimulated activity of the kinase GCN2. Since it was found that GCN2 does not play a role in proteasome recruitment following amino acids starvation (FIGS. 4A and 4B), it was tested whether the eIF2α-ATF4 pathway may be involved in another way. It was observed—via silencing of ATF4—that this transcription factor is required for proteasome export during UPR, but not following amino acids starvation (FIG. 3H). Additionally, overexpression of ATF4 is sufficient to induce proteasome export in the absence of any exogenous stress (FIG. 3I).

### Example 6

Proteasome Translocation is Required for Enhanced Proteolysis of Cytosolic Proteins

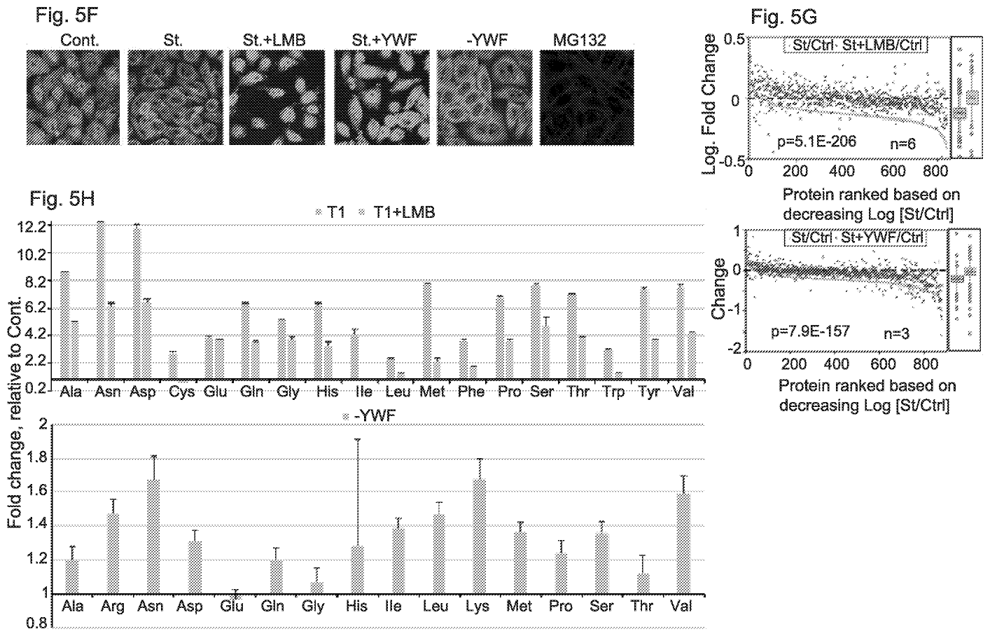

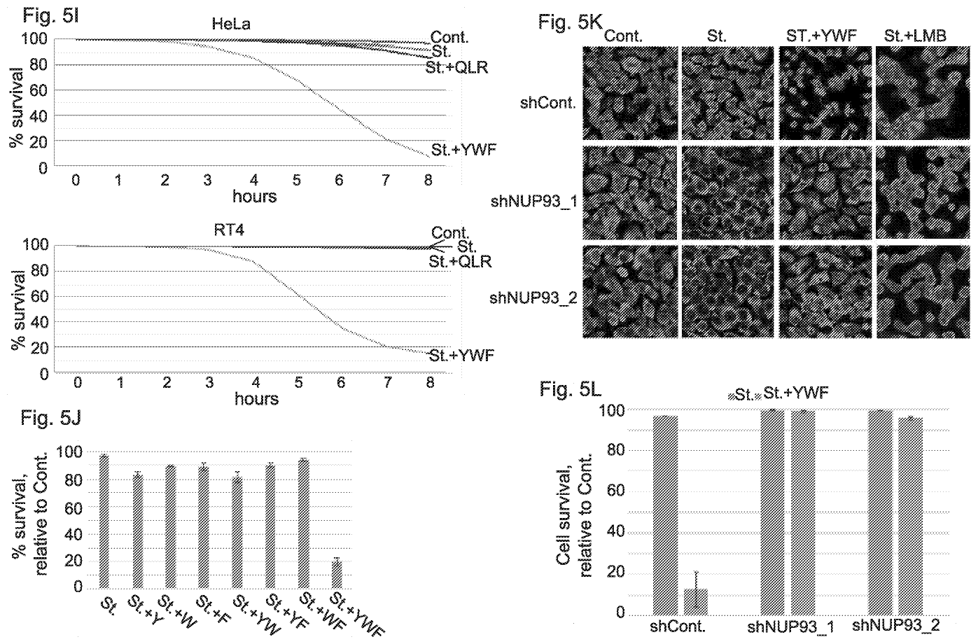

To unravel the role of proteasome translocation under stress, protein breakdown was monitored in fed and starved cells, showing that protein degradation is stimulated ˜2-fold (FIG. 5A) under the amino acids deprivation stress. To link the enhanced proteolysis to the enrichment of the cytosol with nuclear proteasome, LMB was used to inhibit proteasome export to the cytosol, which resulted in inhibition of starvation-induced stimulation of protein breakdown (FIG. 5A). As mentioned above, LMB has no effect on autophagy [Huang, R., et al. (2015). Mol. Cell 57, 456-467], and its inhibitory effect on degradation is most probably due to its effect on proteasome recruitment.

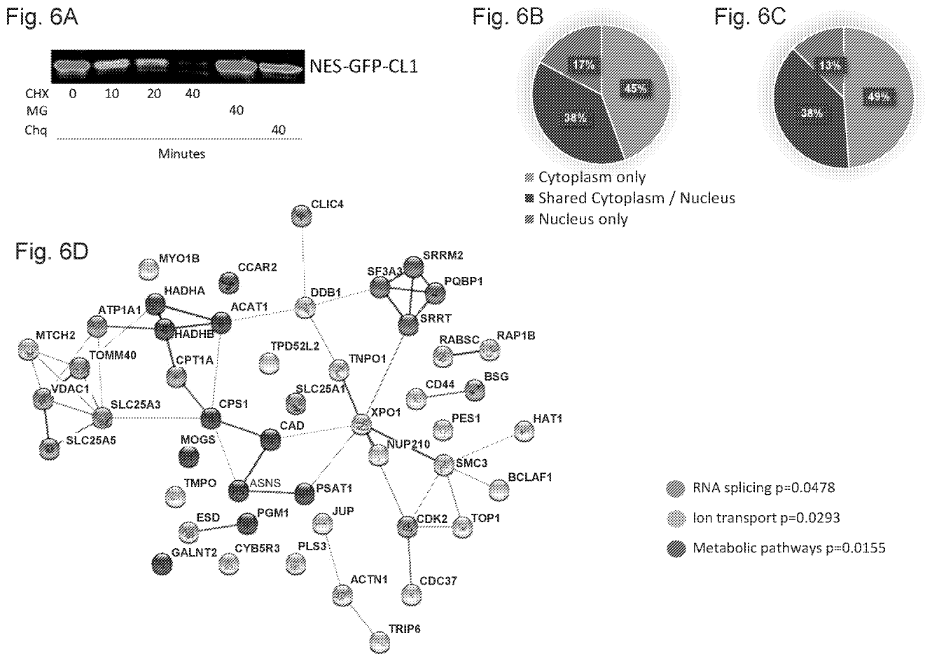

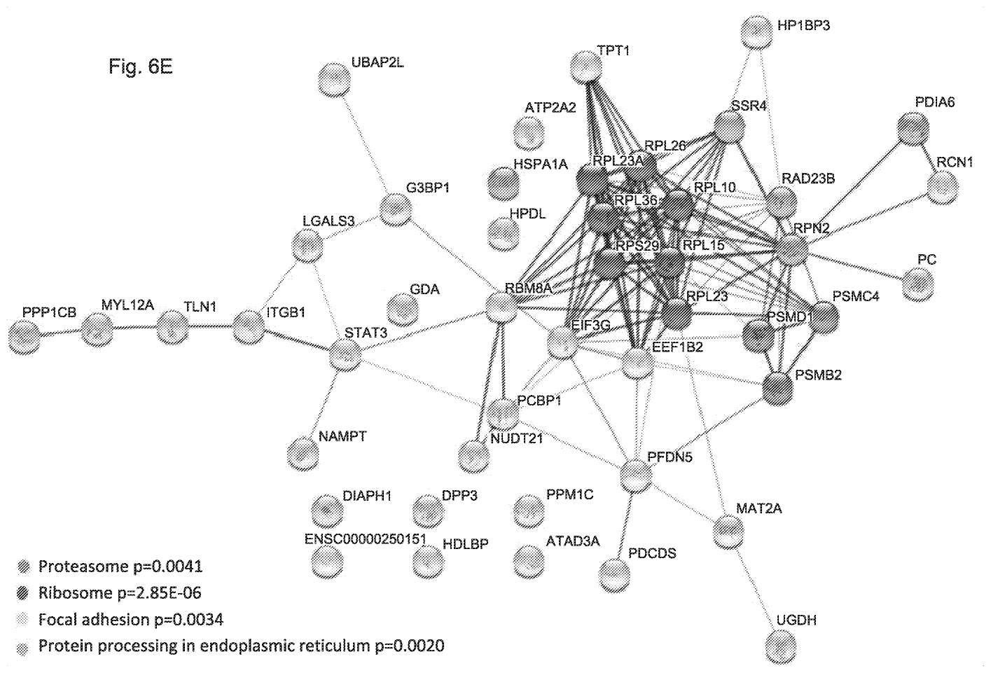

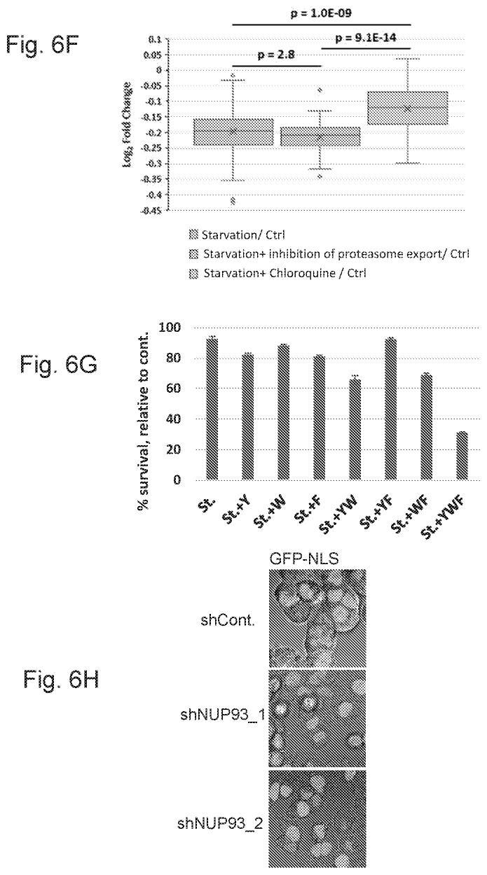

The proteasomal activity was then monitored in both the nuclear and cytosolic fractions, showing that its nuclear activity diminishes following starvation, with a concomitant increase in the cytosolic activity (FIG. 5B). In parallel, the increased cytosolic activity was examined using HMGCS1—a bonafide cytosolic substrate of the proteasome that is degraded following mTOR inhibition [9] and demonstrated that its accelerated degradation under stress is largely dependent on proteasomal export to the cytosol (FIG. 5C). A similar conclusion was attained using the model substrate GFP-CL1—a GFP molecule tagged with the CL1 degron, a motif sensitizing it for rapid ubiquitination and proteasomal destruction [Gilon, T., et al (1998) EMBO J. 17, 2759-2766] which is not subjected to autophagic removal (FIG. 6A). To convert GFP-CL1 to an exclusive cytosolic substrate, an NES was added to it. It was found that while this cytosolic GFP species is degraded under starvation, it is rather stable when proteasomal export is blocked. At the same time, RFP which is removed mostly by autophagy [Berko, D., et al. (2012). Mol. Cell 48, 601-611; Kim, P. K., et al. (2008). Proc. Natl. Acad. Sci. 105, 20567-20574]—is nevertheless degraded (FIG. 5D). It has been shown that during mTOR-mediated stress, ubiquitination is initially upregulated, probably due to increase in ligases activity, followed by a decrease in the level of the generated ubiquitin conjugates due to their proteasomal removal [9]. It appears that preventing export of the proteasome from the nucleus by either YWF or LMB, inhibits degradation and depletion of ubiquitin adducts (FIG. 5E). In contrast, omission of YWF alone resulted in an even lower level of conjugates compared to starved cells. Under these conditions, the proteasome is transported to the cytosol (FIG. 3C), and the degradation of conjugated substrates is accelerated (FIG. 5E). Under these conditions, it was hypothesized that the presence of the remaining seventeen amino acids attenuates the stimulated activity of ubiquitin ligases, resulting in an a priori lower level of conjugates relative to that observed under complete starvation.

By visualizing living cells in the presence of a fluorescent proteasome activity probe [Berkers, C. R., et al. (2007). Mol. Pharm. 4, 739-748], the inventors were able to directly localize the activity of the proteasome, showing once more that starvation, as well as subtraction of YWF, result in translocation of the proteasomal activity to the cytosol. Addition of either LMB or YWF to the starvation medium inhibits the migration of proteasomal activity from the nucleus (FIG. 5F). To assess the effect of proteasome translocation on the stability of the population of cellular proteins, a proteomic assay was conducted, monitoring changes in their level following stimulation and inhibition of proteasome export. The inventors found that upon proteasome translocation stimulated by amino acid starvation, a reduction in the level of ˜900 proteins was observed. This change was prevented by inhibition of proteasomal export using either LMB or YWF (FIG. 5G).

The proteins identified under the different conditions and their dynamics are overlapping to a large extent. Analysis of the proteins which are most affected by inhibition of proteasome translocation by LMB or YWF shows that 83% and 87%, respectively, are either exclusively cytosolic or shared by both the cytoplasm and the nucleus (FIGS. 6B and 6C). Further analysis of the cellular pathways that are enriched in the group of these proteins, reveals key mediators of metabolic pathways (FIG. 6D). That, in contrast to proteins that are unaffected by proteasome dynamics—among which are ribosomes—which are degraded mostly via autophagy (FIG. 6E).

Although autophagy is not affected by LMB [Huang, R., et al. (2015). Mol. Cell 57, 456-467] or YWF (FIG. 3E), it was necessary to further ascertain that the substrates identified by us are mostly dependent on the proteasome for their proteolysis. Monitoring the response of ribosomal proteins—which are cytosolic and are known to be bonafide autophagic substrates—as expected, it was found that the experimental setup identified them as largely subjected to autophagic removal and to a much lesser extent to proteasome dynamics (FIG. 6F).

### Example 7

Proteasome Recruitment to the Cytosol Provides Cells with Amino Acids which are Essential for Cell Survival Under Stress

Next, it was aimed to directly assess the contribution of proteasome translocation to the amino acids pool in stressed cells. To that end, LC-MS was employed to resolve and measure the relative abundance of the different amino acids under the different experimental conditions. In order to measure the change in amino acids pool following proteasome export, the gain in their level was measured following treatment with the mTOR inhibitor Torin1, either in the absence or presence of LMB. While Torin1 stimulates both autophagy and proteasome recruitment, LMB inhibits only the latter. The measurements show that inhibition of proteasome export by LMB significantly inhibits the gain in amino acids produced by Torin1 (FIG. 5H, upper panel), demonstrating that an important role of the translocated proteasome is to replenish the cell with amino acids during short-term deprivation. Similarly, incubation of the cells in a medium containing all amino acids except for YWF which were found to stimulate proteasome translocation with no effect on autophagy (FIG. 3C-3G), results in increased level of all detectable amino acids except for Glu (FIG. 5H, lower panel). Interestingly, Glu was also unaffected by the addition of LMB to Torin1-treated cells, further supporting the validity of these findings. The effect of proteasome translocation, increased cellular proteolysis and supply of amino acids on cell death, was next monitored. Monitoring cell survival via a live time-lapse of two different cell lines, shows that while starvation to the entire repertoire of amino acids is well tolerated, inhibiting proteasome recruitment by the addition of YWF results in their death (FIG. 5I). Assessing the effect of different combinations of these three amino acids on apoptosis—individually as well as in pairs—it was found that the cytotoxic effect of the entire trio is significantly stronger than any other combination. This unexpected synergistic effect demonstrates that they are all needed to induce a maximal effect (FIGS. 5J and 6G).

The inventors then tested whether preventing the entry of the proteasomes to the nucleus during stress in the presence of YWF (that would otherwise drive them to the nucleus and therefore be lethal), would rescue the cell. It was hypothesized that if the proteasome was to remain in the cytosol in a high level, cells would survive. The inventors silenced the nuclear pore complex member NUP93, which was reported to selectively facilitate the nuclear import of Smads, but not that of NLS-harboring proteins [Chen, X. et al., Mol. Cell. Biol. 30, 4022-34 (2010)] and found that it results in a predominant cytosolic distribution of the proteasome (FIG. 5K). Under starvation the proteasome concentration in the cytosol was further increased. Importantly, neither YWF nor LMB had a significant effect on its distribution in cells lacking NUP93 (FIG. 5K) showing the toxic effect of YWF is solely due to its ability to empty the cytosol from the proteasome (FIG. 5L). The inventors validated that NUP93 silencing did not impair NLS-mediated nuclear import (FIG. 6H), demonstrating that the effect of its knockdown on proteasome translocation is not common to all proteins entering the nucleus.

Taken together, these findings further underscore the observation that stress-induced cell death caused by YWF is due to their inhibitory effect on proteasome translocation from the nucleus to the cytosol, and that its migration to the cytosol, where it stimulates proteolysis and replenish the depleted amino acids pool, is essential for cell survival.

### Example 8

Stress-Induced Cytosolic Proteasome Recruitment is Conserved Among Different Species and Tissues

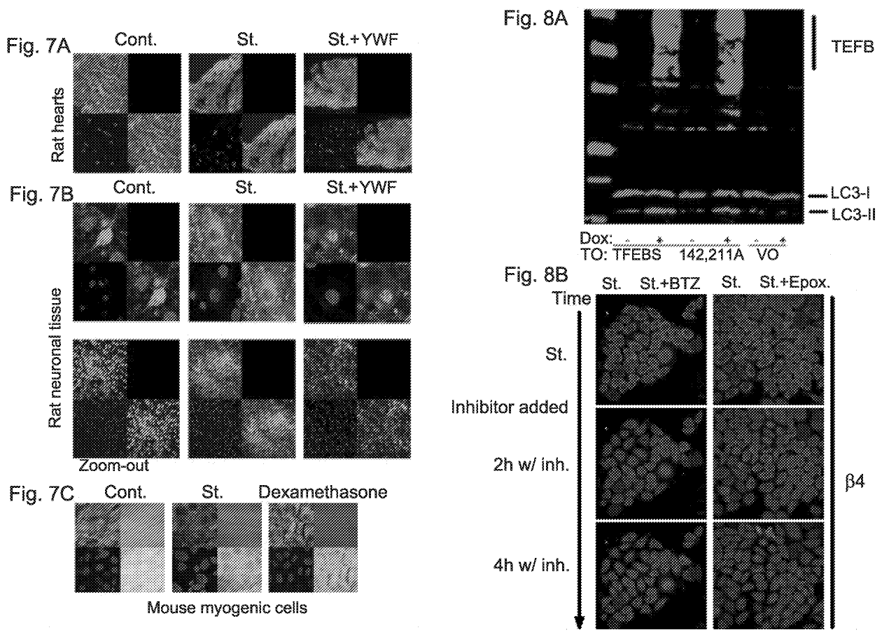

Next, it was important to demonstrate the “universality” of the proteasome response to stress. Using live microscopy of a proteasome activity probe, it was possible to monitor its localization in fresh tissues. As can be seen in FIG. 7A, proteasome activity in an ex vivo perfused rat heart is concentrated in the nucleus, similar to the observation in cultured cells. Subjecting the perfused heart to amino acid starvation, results in voiding of cardiomyocytes' nuclei from their proteasome. YWF prevented this proteasome translocation (FIG. 7A). The same was true for primary cells isolated from rat brains (FIG. 7B).

This phenomenon was then tested in vivo, and the proteasome was monitored in the gut of fruit flies that were starved. As indicated in Example 1, and can be seen in FIG. 1D, localization of the proteasome in control flies was clearly nuclear, whereas in flies fed solely on water and sugar, it was translocated to the cytosol. Further establishing that proteasome export serves a functional role under stress, the effect of corticosteroids was tested, the secretion of which is stimulated under stress—including metabolic stress such as physiologic night sleep fasting. It appears that addition of dexamethasone to differentiated mouse muscle cells, resulted in proteasome translocation as did amino acid starvation (FIG. 7C).

Taken together, the observed preservation of proteasome recruitment under stress in different species and tissues, clearly places it as a fundamental stress-coping mechanism.

### Example 9

The Reciprocal Relationship Between Autophagy and Proteasome Localization and Activity

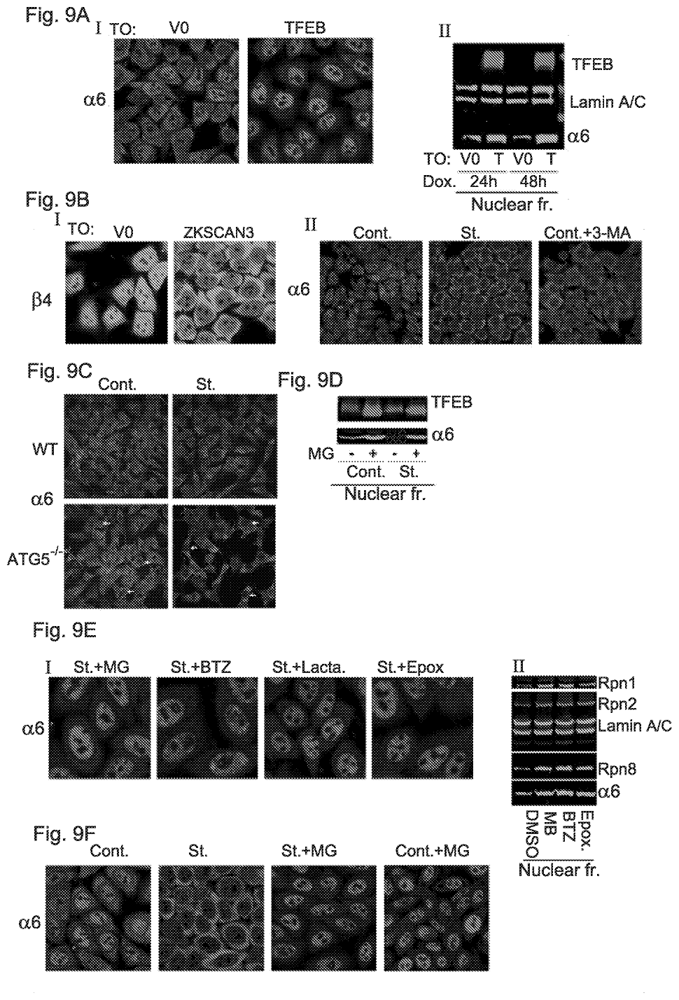

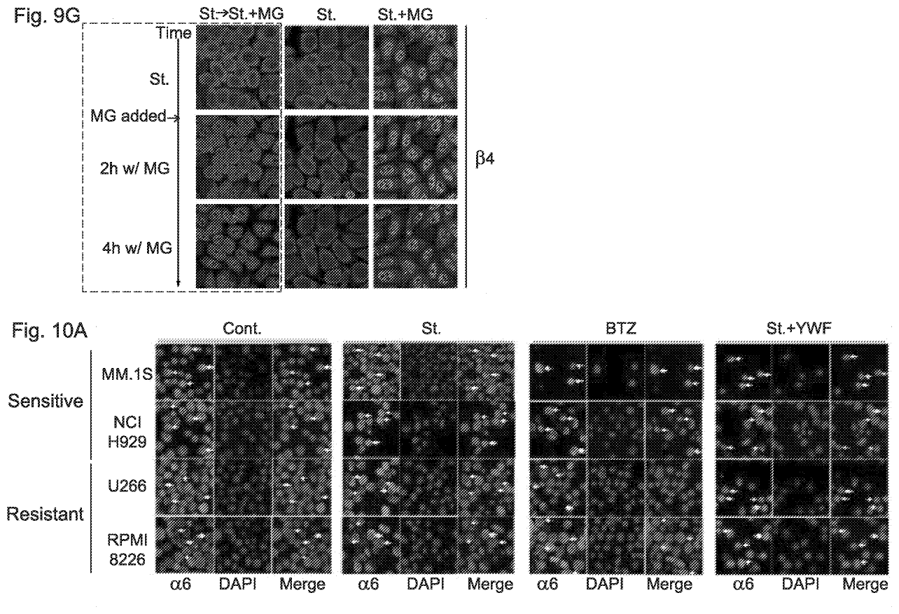

It was shown that the activities of the UPS and autophagy are related temporally [18, 24]. Therefore, it was important to assess whether this reciprocity is also reflected in localization of the proteasome. Several lines of experimental evidence show that this is indeed the case: (1) Upregulation of autophagy stimulated by overexpression of its master regulator TFEB (Settembre, C., et al. (2012) EMBO J. 31, 1095-1108) (FIG. 8A) results in accumulation of the proteasome in the nucleus (FIG. 9A). A constitutively active TFEB was specifically used, where both Ser residues that are phosphorylated by mTOR, a modification that results in inhibition of its transcriptional activity (Settembre, C., et al. (2012) EMBO J. 31, 1095-1108), were mutated to Ala, therefore stimulating autophagy independently of cellular cues; (2) Overexpression of ZKSCAN3, a master transcriptional repressor of autophagy (Chauhan, S. S., et al (2013) Mol. Cell 50, 16-28) led to recruitment of the proteasome to the cytosol (FIG. 9Bi); (3) Inhibition of autophagy by 3-methyl adenine (3-MA) induced the same effect (FIG. 9B ii); (4) Impairment of autophagy by deletion of ATG5 results in accelerated movement of the proteasome to the cytosol under stress (FIG. 9C); (5) Not surprising, inhibition of the proteasome also results in overexpression of TFEB (FIG. 9D), which is in line with the activation of autophagy known to occur under these conditions (Zhu, K., et al (2010) Oncogene 29, 451-462).

Interestingly, it was noted that inhibition of the proteasome is accompanied by its nuclear accumulation, also under starvation (FIG. 9D). This effect was common to several proteasome inhibitors and to both the 19S and 20S sub-complexes (FIG. 9E). The nuclear accumulation of the proteasome following its inhibition even under starvation appears to be active, as addition of the inhibitor to well-nourished cells results in its further nuclear accumulation (FIG. 9F), and addition of the inhibitor after the proteasome already migrated to the cytosol following starvation results in its complete relocation to the nucleus (FIG. 9G and FIG. 8B). The mechanism of this phenomenon is yet to be unraveled. One can hypothesize that inhibition of the proteasome with subsequent decrease in the cellular amino acids pool (that cannot be replenished now by the inhibited enzyme) activates autophagy which stimulates proteolysis, replenishing the depleted pool of amino acids, and as it was demonstrated, leads to accumulation of the proteasome in the nucleus (FIGS. 9A-9B). Whether the mechanism that underlies the relocation is related to activation of TFEB (FIG. 9D) or to a more downstream metabolic effect resulting from stimulated autophagy, is yet to be determined.

### Example 10

Proteasome Inhibitor-Resistant Multiple MM Cells Exhibit Cytosolic Distribution of the Proteasome Under Basal Metabolic Conditions, which Plays an Important Role in their Resistance

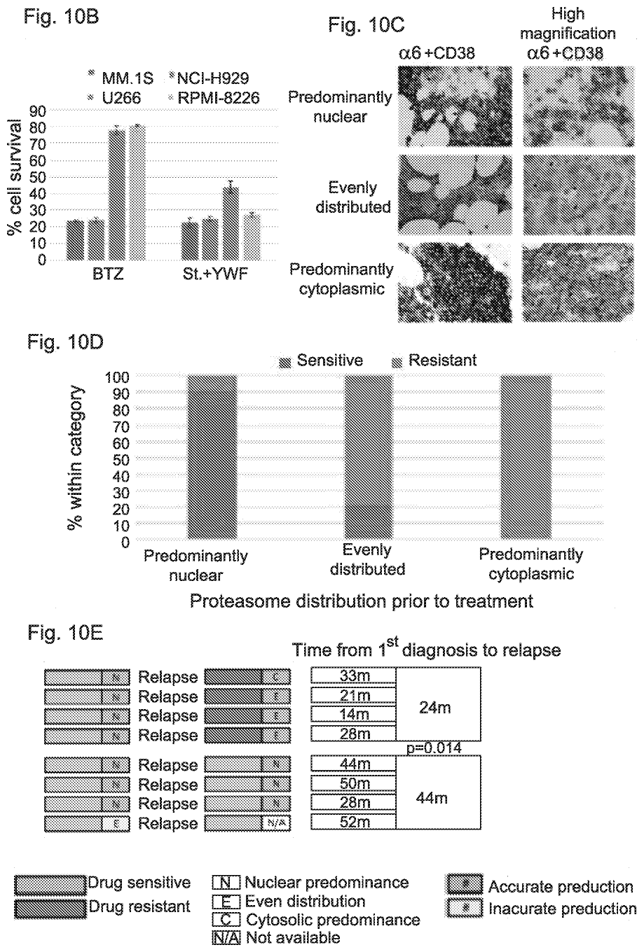

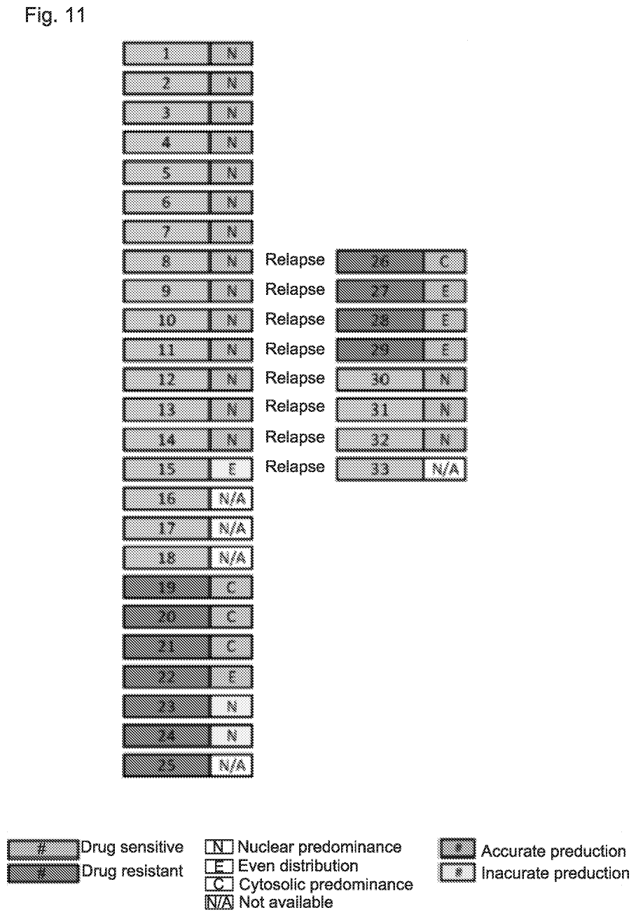

Proteasome inhibitors are used as first line of treatment in MM—a malignant clonal expansion of immune plasma cells. Along with other drugs that also exert some of their effect through the UPS, they have revolutionized the management and prognosis of patients. Nevertheless, patients' response to treatment spans a wide range, and after a favorable outcome—practically all patients relapse at some point, despite being on a maintenance treatment [19]. The mechanism underlying proteasome inhibition resistance has remained elusive [19], and has been demonstrated also in patient-derived cultured MM cells [25]. Monitoring the proteasome localization in Bortezomib-resistant MM cultured cells, it was found that—unlike their sensitive counterparts—the proteasome shows a loss of nuclear preference (FIG. 10A). To test whether the resistance to proteasome inhibitor can be attributed, at least in part, to the high level of proteasomes in the cytosol, and to attempt to overcome it, YWF was used in order to force the cytosolic proteasome into the nucleus during starvation. The results show that unlike proteasome inhibitors, which induce apoptosis only in the sensitive MM cells, forced nuclear sequestration of the proteasome following addition of YWF induces apoptosis also in Bortezomib-resistant cells (FIGS. 10A and 10B). These findings suggest that the ability of cells to evade the normal regulation of proteasome dynamics and maintain the proteasome in the cytosol under different conditions, contributes to their tolerance to treatment and probably aggressiveness. The ability of YWF to enforce a predominant nuclear localization also in resistant cells opens a potential therapeutic approach for treatment of such patients.

Interestingly, the sensitive MM cells demonstrate an even stronger nuclear preference of the proteasome, relative to cells of other tissues (FIG. 1A, FIGS. 2A and 2B), and fail to recruit the proteasome to the cytosol under starvation, a basic protective mechanism other cells are employing during stress (FIG. 10A). These observations may provide a possible mechanistic reasoning as to why this malignancy has turned out to be a target for proteasome-inhibiting drugs in the first place. It seems that these cells have an unusual low reserve of cytosolic proteasome which is probably required for the degradation of the misfolded proteins that arise from the vast quantities of the immunoglobulin molecules they synthesize. This, along with the paucity of cytosolic proteasome, sets their threshold for stress intolerability lower than other cells.

### Example 11

Proteasome Dynamics Offer a Predictive Tool for the Efficacy of Treatment with Proteasome Inhibitors in Newly Diagnosed MM Patients

Based on our results in cultured cells, it was hypothesized that the basal proteasome distribution in newly diagnosed MM patients can provide a predictive tool as for their susceptibility to proteasome inhibitors.

To test this hypothesis, proteasome distribution was blindly assessed in bone marrow biopsies from MM patients before initiation of treatment. Only later, the findings were correlated with the patients' response to the treatment with proteasome inhibitors.

Similar to cultured cells, the proteasome in the biopsies was found in different patients—to display different patterns of sub-cellular distribution: it was either predominantly nuclear or cytosolic, or was evenly distributed between the two compartments (FIG. 10C). Comparing the histopathological findings with the clinical outcome shows that, when the proteasome was mostly nuclear—90% of the patients were responsive to the treatment. In striking contrast, loss of nuclear preference predicted with high likelihood that the disease is drug-resistant: 80% of the patients with even distribution—and 100% of the patients that showed cytosolic predominance of the proteasome—were resistant to treatment (FIG. 10D, and the schematic representation in FIG. 11). Importantly, in the group of patients that relapsed, and a 2nd biopsy was taken prior to resuming treatment, all the patients who turned out at that stage to be resistant to the treatment, have also lost their previous nuclear dominance of the proteasome (observed when they were sensitive to the drug). In contrast, all biopsies from patients who remained drug-sensitive also after a relapse, have maintained a nuclear dominance of the proteasome. Noteworthy, the two groups differed significantly also in their remission period that preceded the relapse: patients who became drug-resistant (concomitantly with a loss of nuclear proteasome localization), had a mean interval of 24 months between their first diagnosis and the relapse. In contrast, those who remained sensitive to the drug (while also maintaining a nuclear proteasome dominance), had a mean remission interval of 44 months (FIG. 10E). Taken together, our findings in tissue culture and in patients unravel one of the mechanisms responsible for drug resistance in MM and may provide care takers with a useful predictive tool as for the efficacy of treatment.

### Example 12

Proteasome Recruitment is Essential for Tumor Growth In Vivo, and its Inhibition Results in Cell Death and Reduction in Tumor Size

The possible effect of YWF on proteasome dynamics was next examined in tumor models. It was hypothesized that the stress gradient, which is inherent to solid tumors, where their core is characteristically more hypoxic and relatively short in nutrients compared to the periphery (Minchinton, A. I., and Tannock, I. F. (2006) Nat. Rev. Cancer 6, 583-592), serves as a stimulus for proteasome migration. This hypothesis is in line with the finding that in addition to nutrient shortage, hypoxia also induces proteasome recruitment (FIG. 1H).

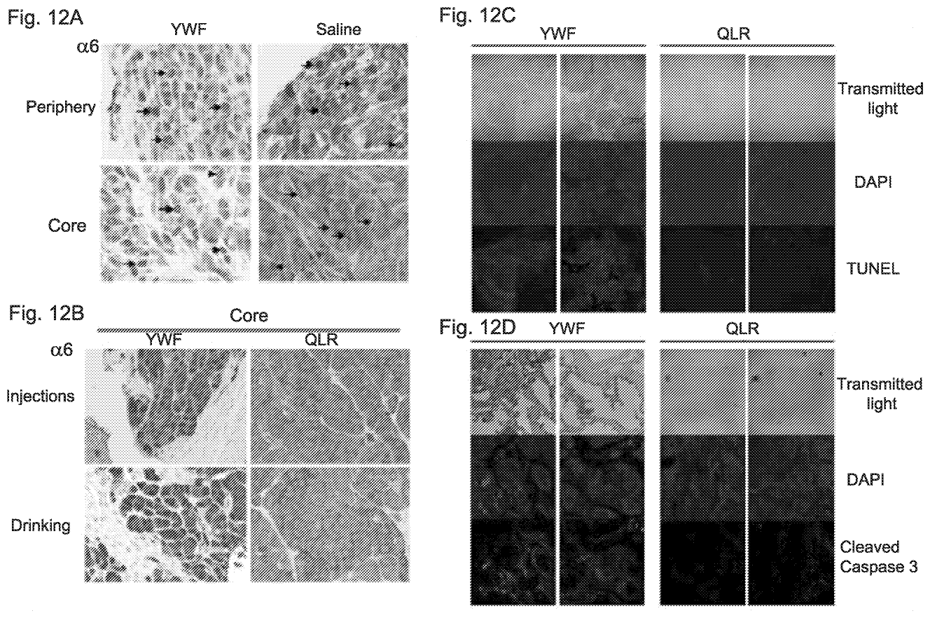

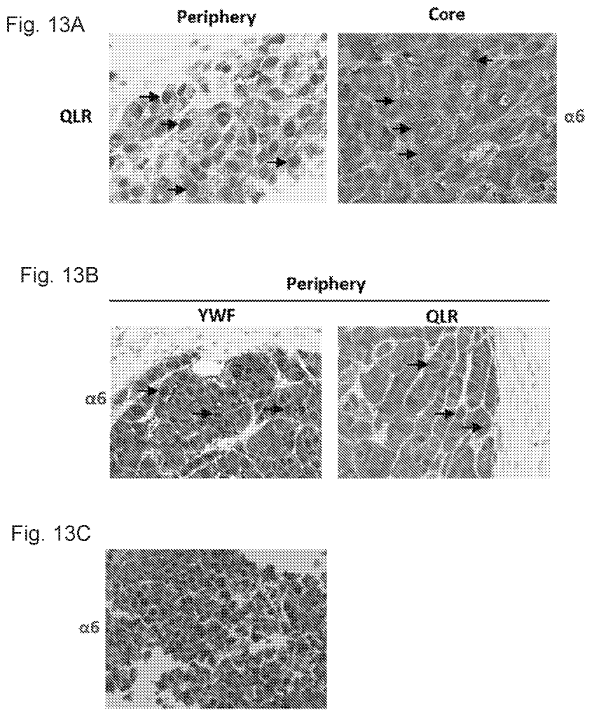

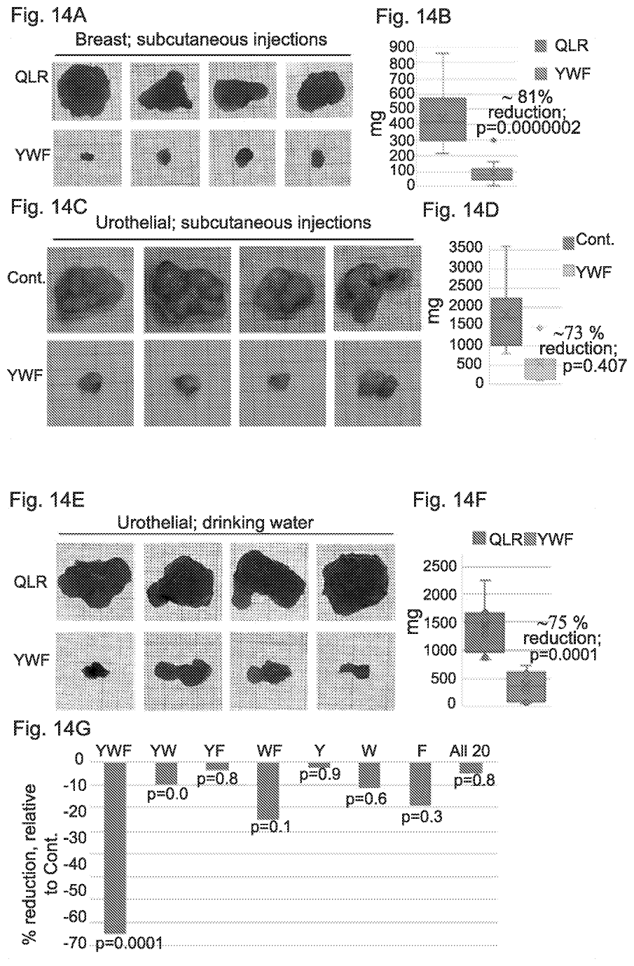

Using human breast and urothelial tumor models in mice, the inventors showed that on the non-stressed periphery of the tumor, the proteasome is largely nuclear (FIG. 12A). That, in contrast to its core where the proteasome is more enriched in the cytosol (FIG. 12A). Following injection of YWF (subcutaneously to the tumor bed), a clear nuclear localization of the proteasome was observed also in the tumor's core (FIGS. 12A and 12B). In contrast, injection of QLR did not affect proteasome distribution (FIGS. 12B and 13A, 13B). Importantly, administration of YWF orally —via the drinking water—had the same effect on proteasome localization as subcutaneous injections (FIG. 12B).

Next, it was important to demonstrate that “locking” the proteasome in the nucleus during stress has a cytotoxic effect on tumors. Therefore, tumors were stained for the apoptotic markers TUNEL and cleaved-Caspase3. As shown by FIG. 12, concomitantly with their induction of proteasome nuclear accumulation, YWF exerted also a wide cytotoxic effect on the stressed tumor cells (FIGS. 12C and 12D). These parts of the tumor also show characteristic architecture of damaged tissue, necrosis, and fibrosis (FIGS. 12C, 12D and 13C). As expected, sporadic dying cells are visible also in the control group (QLR), yet the magnitude of apoptosis and tissue necrosis is much higher in the core of YWF-treated tumors (FIGS. 12C and 12D).

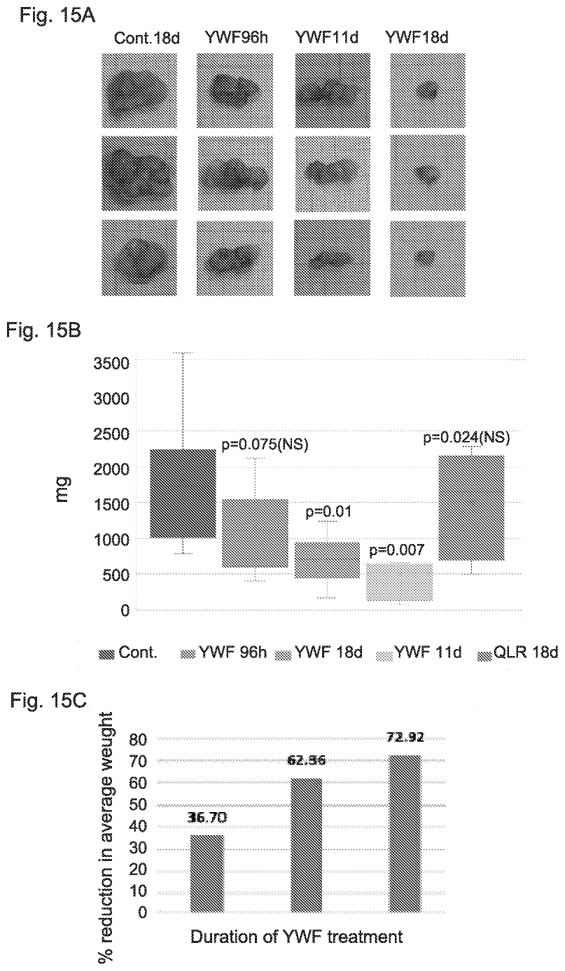

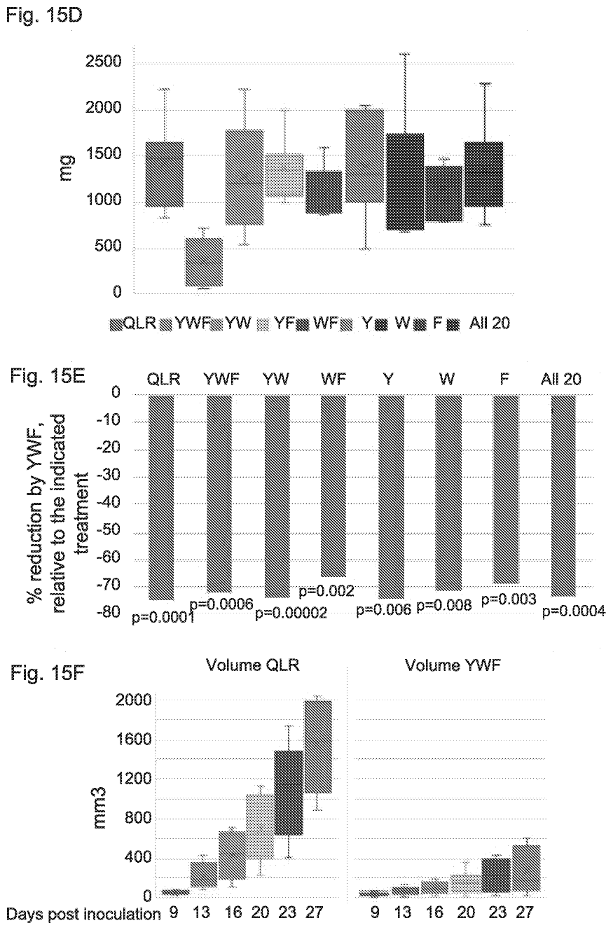

Observing the tumors macroscopically and comparing their weight, the inventors showed that the effect of YWF at the cellular level (i.e., proteasome nuclear retainment and apoptosis) is accompanied also by a significant reduction of up to ˜80% in tumor size compared to control tumors (FIG. 14A-14E and 15D). Importantly, YWF are efficient inhibitors of tumor growth regardless of their route of administration (subcutaneously or per os in drinking water) or the type of tumor that was tested (FIG. 14A-14E). YWF are effective even when given late in the course of tumor development, in which case tumors were allowed to reach a significantly large size prior to the initiation of treatment (FIG. 15A-15C).

In light of the findings that in cultured cells, all three aromatic amino acids are required for a substantial inhibition of proteasome recruitment and subsequent apoptosis (FIG. 5J), it was aimed to check the same in a tumor model. Therefore, mice were treated through their drinking water, with all combinations of Tyr, Trp, and Phe—individual amino acids as well as all possible pairs. As clearly shown by FIGS. 14F and 14G, only the three of them together have induced a significant reduction in tumor size. Moreover, the trio displaying a significant synergistic effect, was far superior to any other combination, when directly compared (FIG. 15E, 15F). Importantly, administration of all twenty amino acids had no effect on tumor growth (FIG. 14F).

In summary, the findings of the present disclosure unravel a key role for proteasome dynamics as a stress-coping mechanism in solid tumors, which has potential therapeutic implications for solid and hematological malignancies.

### Example 13

Stress-Induced Proteasome Translocation is Prevented by D-YWF, and by Mixture of the Both Isomers, L-YWF and D-YWF

As shown in FIGS. 3 and 5, L-YWF affect proteasome translocation of starved cells. The effect of D-isomers of YWF, was next examined on starved cells. As shown in FIG. 16, also the D-isomers clearly inhibit proteasome recruitment, however less efficiently than their L counterparts. Aromatic amino acids such as Phe, Trp, Tyr, and His were previously reported to form a wide range of nanostructures including fibers, nanotubes, nanoribbons, twisted nanosheets, dendritic structures, etc., depending on the self-assembly conditions. These nanofibrillar structures demonstrated marked cytotoxicity. By employing D-enantiomers, Gazit et al., (ACS Nano 2020, 14, 2, 1694-1706), recently demonstrated the critical role of amino acid chirality in the self-assembly process. More specifically, racemic mixture of the L- and D-isomers prevented the formation of these nanofibrillar structures by each individual enantiomer. Thus, if the observed lethality of L-YWF in starved cells is connected with formation of these structures, a racemic mixture of, should prevent this effect. The staved cells were therefore treated with a racemic mixture of the L-YWF and D-YWF. As sown in the lower panel of FIG. 16, the racemic mixture efficiently inhibits proteasome recruitment, indicating that the lethality of L-YWF is not connected with formation of nanofibrillar structures.

### Example 14

Scanning Tumor Biopsies for the Localization of the Proteasome to Identify Candidate Responders for YWF Treatment-Providing a Tool for Tailored Treatment

A pathological survey of a broad array of biopsies from human tumors (e.g., liver-biliary, brain, lung, pancreas, colorectal, diffuse large B cell lymphoma [DLBCL], breast, and ovary), is next scanned by the inventors for localization of the proteasome. This analysis serves as an indicator for tumors that can be sensitive to treatment with selective inhibitors of proteasome translocation (e.g., the YWF), and is further used as a prognostic tool for monitoring the clinical outcome and success of the available treatment.

Proteasome localization is determined for each sample as described in the previous examples and in the experimental procedures. Tumor tissues displaying a cytosolic distribution of the proteasome, or equal distribution, at least in part of the tumor cell of the examined tumor tissue, are classified as candidate responders for a selective inhibitor of proteasome translocation, such as the YWF triad of the invention. Candidate responders are further evaluated as discussed herein after.

Next, patient-derived xenografts (PDXs) of tumors, are used to corroborate in vivo the predictions that were made based on the pathological and clinical findings, to further evaluate the candidate responders. More specifically, fresh surgical samples of patients—PDXs are generated in SCID mice. Mice are next treated with a selective inhibitor of proteasome translocation, for example, YWF. Correlation between the localization of the proteasome and response to treatment are made.

This method serves as a proof-of-concept that proteasome distribution is indeed a valid patient-specific indicator for a tailored treatment. This model further provides an access to potential mechanistic clues as well as for target(s) and marker(s) identification. To that end the healthy mouse tissue along with its corresponding implanted human tumor are subjected to transcriptomic analysis. These tissues along with the mouse plasma are also subjected to metabolomic analysis.

### Example 15

The Efficacy of YWF in the Treatment of a Spontaneous, Endogenic Tumors in Mice

Encouraged by the findings that YWF administration can strongly inhibit tumor growth in mouse xenograft models, the inventors next evaluated the effect of the riad of the invention on tumors rising from an endogenous tissue in immune competent animals. The APC^{fl/fl} CDX2-Cre-ER model was therefore used. In this tumor model, knockout (KO) of the Adenomatous Polyposis Coli (APC) gene is induced selectively in the intestines, via the administration of tamoxifen—an estrogen receptor modulator. APC is a key tumor suppressor gene, and mutations in this gene are found in most cases of colon cancer in human patients.

This model allows monitoring the growth of tumors that (1) arise from normal tissues due to cellular dysregulation, as in real cases of cancer; (2) recapitulate the molecular chain of events as in patients; (3) grow at the true anatomical site within the organism; and (4) develop in an animal with an intact immune system, which is known to play a role in tumorigenesis.

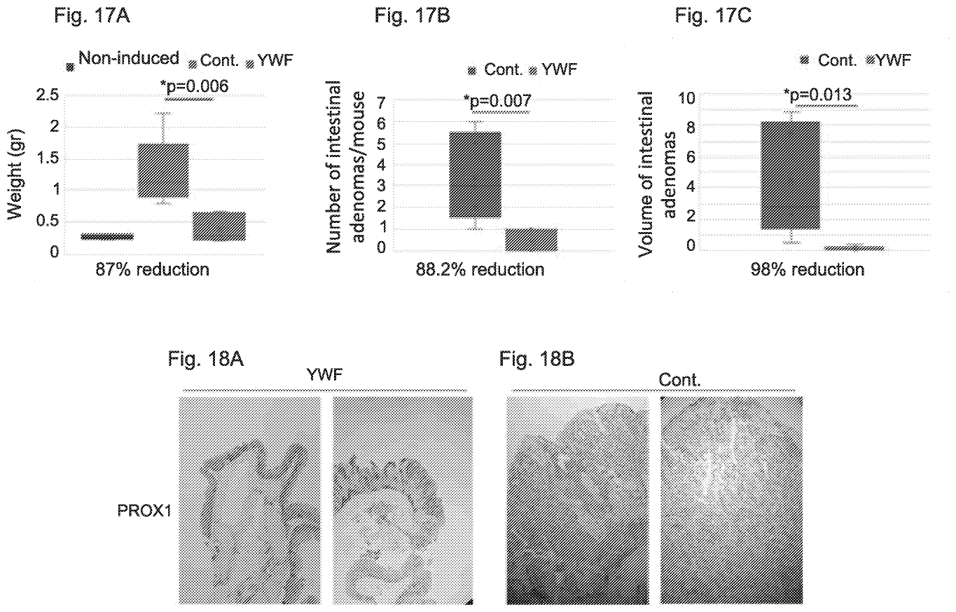

Following induction and development of tumors in the gastrointestinal tract, mice were treated with YWF in their drinking water, as previously described for the xenograft models. As can be seen in FIG. 17, YWF treatment resulted in a significant reduction of tumor burden, as reflected by the following parameters:

First, as shown in FIG. 17A, in the cecum, the developed tumors are forming a neoplastic conglomerate, which is assessed by weighing the cecum. The excess weight—relative to the weight of a normal cecum in a tumor-free animal, represents the extent of tumor growth. Relative to the placebo group, YWF reduced tumor growth in the cecum in 87%.

Second, as shown in FIG. 17B, along the intestine, distinct tumors are forming, and their number is indicative for the extent of the disease. Relative to the control group, YWF reduced the number of intestinal tumors in >88%.

Third, as shown in FIG. 17C, in addition to their number, each intestinal tumor is measured using a caliper, and its volume is calculated. Summing the volumes of all such tumors in a single animal gives the total volume as an indication for tumor burden. YWF reduced the average tumor volume load in 98%, compared with the placebo group.

The inventors found that the YWF shrinking effect on tumors is visible also microscopically, and in some cases the treatment eliminated them almost entirely. In contrast, in the placebo group large tumors were clearly visible, virtually obscuring the normal gut tissue (FIGS. 18A and 18B). The samples were stained using PROX1, a marker for high-grade dysplasia, further demonstrating that YWF strongly inhibits the growth of cancer (FIG. 18A), as compared to control placebo group (FIG. 18B).

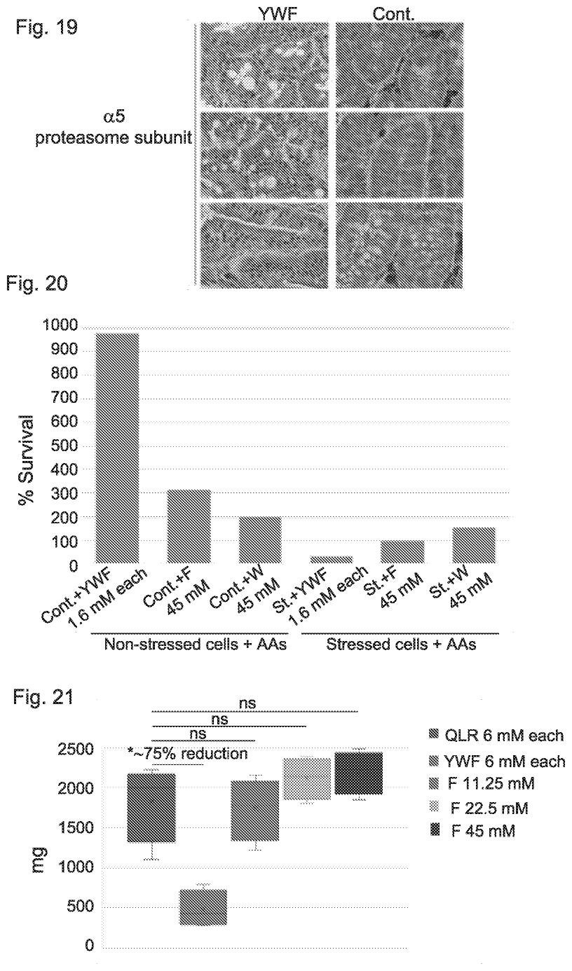

To establish the link between proteasome localization and the observed inhibition of tumor growth, as was shown in xenografts, the samples were stained for the proteasome subunit α5. As can be seen in FIG. 19, the proteasome largely translocate to the cytosol of cells within the tumors of the placebo group, while the YWF treatment sequesters it in the nucleus.

Taken together, these results in the endogenous APC colon cancer model recapitulate those obtained in the xenograft models, underscoring the validity of the therapeutic approach of the present disclosure, as well as the relative universality of its application, by means of the various tumor types which may be treated using YWF.

### Example 16

Assessing Toxicity and Efficacy of the YWF Composition, Relative to High-Dose Treatment of L-Phenylalanine Treatment

The inventors next evaluated the effect of treatment with the YWF triad of the invention (YWF at a concentration of 1.6 mM/each) as compared with high concentration of phenylalanine (45 mM F, as disclosed by WO2015137383A1 [14]), therefore, the medium of cultured cells was supplemented with the appropriate amino acid(s). Since tumor cells are inherently stressed—due to high metabolic demands and poor perfusion of nutrients and oxygen, starved cells in culture were used in order to simulate the effect of the different treatments. Similarly, to simulation of the effect of each treatment on “normal” tissues in vivo—which are not stressed—the same amino acid(s) were added to non-starved cells in culture.

As can be seen in FIG. 20, the YWF mixture is the most effective treatment against the stressed cancer cells, among those tested. That, despite its relative low concentration, which points out to the synergistic effect of the three aromatic amino acids. Importantly, such low concentrations result in minimal (if any) adverse effects to non-stressed cells. In contrast, the treatment with high concentration of phenylalanine (45 mM F, [14]) was highly lethal also to non-stressed cells. Its toxicity towards both stressed and non-stressed cells shows that this approach is non-selective, unlike the low-dose mixture of YWF of the present disclosure.

To conclude, the YWF mixture of the present invention not only displays the highest efficacy against the stressed cells (mimicking tumor cells in the whole organism), but is also the most selective treatment—with virtually no deleterious effect to the non-stressed cells (mimicking non-cancerous tissues in patients).

In addition to the assessment of phenylalanine (F) at 45 mM, the effect of high concentration (45 mM) of an additional aromatic amino acid residue, tryptophan (W), was next evaluated. As seen in FIG. 20, the results are similar to those obtained for 45 mM F, underscoring the lack of selectivity of a single aromatic amino acid at a high dose, and the significant synergism (and lack of toxicity) of the triad—when administrated together. Of note, is that tyrosine (Y) is not soluble to the extent of 45 mM, and was therefore not tested separately, as were W and F.

As far as results from cultured cells are indicative, these data render the YWF mixture of the present invention superior, and therefore clearly preferable for use. Moreover, these data clearly suggest that treatment using 45 mM of F (or W) is non-selective and may harm stressed and non-stressed cells alike.

The inventors next aimed to assess the anti-tumorigenic effect of the above treatments in vivo, using a tumor model in mice. Following tumor formation, each group was treated with a different treatment, and the size of tumors was eventually compared relative to the control group (QLR). As clearly seen in FIG. 21, the low-dose YWF combination significantly inhibited tumor growth by about 75%, while F alone did not result in any benefit even when given at a concentration of 45 mM.

In summary, treatment using 45 mM F, is clearly inferior to the mixture of YWF of the preset disclosure at 1.6 mM/each in eliminating stressed cancerous cells in culture. Still further, the treatment with high dose of phenylalanine (45 mM F) is non-selective, and therefore harmful to non-stressed cells, while YWF are selective and non-harmful. More importantly, treatment with high-dose phenylalanine (45 mM F) displayed no effect on tumor growth in a xenograft mouse model, unlike the YWF triad of the present invention which significantly reduce tumor size.

These comparative experiments clearly show the superiority of the YWF triad and demonstrate the feasibility of therapeutic uses thereof.

1-45. (canceled)

46. A mammalian target of rapamycin (mTOR) agonist comprising a combination of at least two aromatic amino acid residues or any mimetics thereof, any compound that modulates directly or indirectly at least one of the levels, stability and bioavailability of at least one of said aromatic amino acid residues, any combinations or mixtures thereof or any vehicle, matrix, nano- or micro-particle thereof said mTOR agonist comprising at least two of:
(a) at least one tyrosine (Y) residue, any mTOR agonistic tyrosine mimetic, any salt or ester thereof any multimeric and/or polymeric form of said tyrosine residue and/or of said mTOR agonistic tyrosine mimetic, and any combinations or mixtures thereof;
(b) at least one tryptophan (W) residue, any mTOR agonistic tryptophan mimetic, any salt or ester thereof, any multimeric and/or polymeric form of said tryptophan residue and/or of said mTOR agonistic tryptophan mimetic, or any combination or mixture thereof; and
(c) at least one phenylalanine (F) residue, any mTOR agonistic phenylalanine mimetic, any salt or ester thereof any multimeric and/or polymeric form of said phenylalanine residue and/or of said mTOR agonistic phenylalanine mimetic, and any combinations or mixtures thereof.

47. The mTOR agonist according to claim 46, comprising:
(a) at least one tyrosine residue, any mTOR agonistic tyrosine mimetic, any salt or ester thereof any multimeric and/or polymeric form of said tyrosine residue and/or of said mTOR agonistic tyrosine mimetic, and any combinations or mixtures thereof;
(b) at least one tryptophan residue, any mTOR agonistic tryptophan mimetic, any salt or ester thereof any multimeric and/or polymeric form of said tryptophan residue and/or of said mTOR agonistic tryptophan mimetic, or any combination or mixture thereof; and
(c) at least one phenylalanine residue, any mTOR agonistic phenylalanine mimetic, any salt or ester thereof any multimeric and/or polymeric form of said phenylalanine residue and/or of said mTOR agonistic phenylalanine mimetic, and any combinations or mixtures thereof.

48. A composition comprising as an active ingredient at least one mTOR agonist according to claim 46, or any vehicle, matrix, nano- or micro-particle thereof, optionally in at least one dosage form, said composition optionally further comprises at least one pharmaceutically acceptable carrier/s, excipient/s, auxiliaries, and/or diluent/s.

49. The composition according to claim 48, wherein said composition comprises:
(a) at least one tyrosine residue, any mTOR agonistic tyrosine mimetic, any salt or ester thereof any multimeric and/or polymeric form of said tyrosine residue and/or of said mTOR agonistic tyrosine mimetic, and any combinations or mixtures thereof;
(b) at least one tryptophan residue, any mTOR agonistic tryptophan mimetic, any salt or ester thereof any multimeric and/or polymeric form of said tryptophan residue and/or of said mTOR agonistic tryptophan mimetic, or any combination or mixture thereof; and
(c) at least one phenylalanine residue, any mTOR agonistic phenylalanine mimetic, any salt or ester thereof any multimeric and/or polymeric form of said phenylalanine residue and/or of said mTOR agonistic phenylalanine mimetic, and any combinations or mixtures thereof.

50. The composition according to claim 48, wherein at least one of:
(a) said at least one mTOR agonist is formulated as an oral dosage form or as an injectable dosage form;
(b) said oral dosage form is in a formulation adapted for add-on to a solid, semi-solid or liquid food, beverage, food additive, food supplement, medical food, drug and/or a pharmaceutical composition.

51. A kit comprising the mTOR agonist according to claim 46, said kit comprises at least two of:
(a) at least one tyrosine residue, any mTOR agonistic tyrosine mimetic, any salt or ester thereof any multimeric and/or polymeric form of said tyrosine residue and/or of said mTOR agonistic tyrosine mimetic, and any combinations or mixtures thereof, optionally, in a first dosage form;
(b) at least one tryptophan residue, any mTOR agonistic tryptophan mimetic, any salt or ester thereof any multimeric and/or polymeric form of said tryptophan residue and/or of said mTOR agonistic tryptophan mimetic, or any combination or mixture thereof optionally, in a second dosage form; and
(c) at least one phenylalanine residue, any mTOR agonistic phenylalanine mimetic, any salt or ester thereof any multimeric and/or polymeric form of said phenylalanine residue and/or of said mTOR agonistic phenylalanine mimetic, and any combinations or mixtures thereof, optionally, in a third dosage form, optionally, said kit further comprises at least one ubiquitin proteasome system (UPS) modulating agent, and/or at least one therapeutic agent, optionally, in a fourth dosage form.

52. A method for treating, inhibiting, reducing, eliminating, protecting or delaying the onset of at least one condition or at least one pathologic disorder associated with cytosolic proteasomal localization and/or activity in a subject, the method comprising the step of administering to said subject an effective amount of at least one mTOR agonist according to claim 46, said mTOR agonist comprising at least one aromatic amino acid residue, any mTOR agonistic mimetic thereof any salt or ester thereof any multimeric and/or polymeric form of said at least one aromatic amino acid residue and/or of said mTOR agonistic mimetic, any compound that modulates directly or indirectly at least one of the levels, stability and bioavailability of said at least one aromatic amino acid residue, any combinations or mixtures thereof any vehicle, matrix, nano- or micro-particle thereof any dosage form thereof or any composition or kit comprising said at least one mTOR agonist.

53. The method according to claim 52, wherein said at least one mTOR agonist comprises:
(a) at least one tyrosine residue, any mTOR agonistic tyrosine mimetic, any salt or ester thereof any multimeric and/or polymeric form of said tyrosine residue and/or of said mTOR agonistic tyrosine mimetic, and any combinations or mixtures thereof, optionally, in a first dosage form;
(b) at least one tryptophan residue, any mTOR agonistic tryptophan mimetic, any salt or ester thereof any multimeric and/or polymeric form of said tryptophan residue and/or of said mTOR agonistic tryptophan mimetic, or any combination or mixture thereof optionally, in a second dosage form; and
(c) at least one phenylalanine (F) residue, any mTOR agonistic phenylalanine mimetic, any salt or ester thereof any multimeric and/or polymeric form of said phenylalanine residue and/or of said mTOR agonistic phenylalanine mimetic, and any combinations or mixtures thereof, optionally, in a third dosage form.

54. The method according to claim 52, wherein said subject is further administered with at least one of, at least one UPS-modulating agent and/or at least one therapeutic agent, prior to, after and/or simultaneously with administration of said at least one mTOR agonist.

55. The method according to claim 52, wherein at least one of:
(a) said at least one mTOR agonist is formulated as an oral dosage form or as an injectable dosage form; optionally, said oral dosage form is in a formulation adapted for add-on to a solid, semi-solid or liquid food, beverage, food additive, food supplement, medical food, drug and/or a pharmaceutical composition;
(b) said at least one mTOR agonist is administered orally to said subject; and
(c) said subject is and/or was subjected to dietary restriction of amino acids.

56. The method according to claim 52, wherein said pathologic disorder associated with cytosolic proteasomal localization and/or activity is at least one proliferative disorder and/or at least one protein misfolding disorder or deposition disorder; optionally, wherein at least one of:
(a) said proliferative disorder is at least one of a benign or malignant solid and non-solid tumor; and
(b) said protein misfolding disorder is amyloidosis and any related conditions.

57. A method for modulating a biological process associated directly or indirectly with proteasome dynamics in at least one cell and/or in a subject, the method comprising the step of contacting said at least one cell and/or administering to said subject a therapeutically effective amount of at least one mTOR agonist according to claim 46, or any combinations or mixtures thereof, any vehicle, matrix, nano- or micro-particle thereof any dosage form thereof or any composition or kit comprising said mTOR agonist.

58. A method for treating, inhibiting, reducing, eliminating, protecting or delaying the onset of at least one of at least one proliferative disorder and/or at least one protein misfolding disorder in a subject in need thereof, and/or for determining a personalized treatment regimen for a subject suffering from a pathologic disorder, the method comprising the steps of:
(a) determining proteasome subcellular localization in at least one cell of at least one biological sample of said subject, or in any fraction of said cell;
(b) classifying said subject as:
(i) a responder subject to a treatment regimen comprising at least one UPS-modulating agent, if proteasome subcellular localization is predominantly nuclear; or
(ii) a drug-resistant subject if proteasome subcellular localization is cytosolic; and
(c) selecting a treatment regimen based on said responsiveness, thereby treating said subject with the selected treatment regimen, and/or determining a personalized treatment regimen for said subject.

59. The method according to claim 58, wherein step (c) comprises:
(i) administering to a subject classified as a responder, an effective amount of at least one UPS-modulating agent, any combinations thereof or any compositions comprising the same; or
(ii) administering to a subject classified as a drug-resistant, an effective amount of at least one mTOR agonist, or any combinations thereof optionally, with at least one UPS-modulating agent and/or at least one therapeutic agent.

60. The method according to claim 59, wherein said at least one mTOR agonist comprises at least one aromatic amino acid residue, any mTOR agonistic mimetic thereof, any salt or ester thereof any multimeric and/or polymeric form of said at least one aromatic amino acid residue and/or of said mTOR agonistic mimetic, any compound that modulates directly or indirectly at least one of the levels, stability and bioavailability of said at least one aromatic amino acid residue, any combinations or mixtures thereof any vehicle, matrix, nano- or micro-particle thereof, any combinations or mixtures thereof any composition or kit comprising the same.

61. The method according to claim 58, wherein said subject is and/or was subjected to a treatment regimen comprising at least one UPS-modulating agent and is monitored for disease progression, the method comprising the step of:
(a) determining proteasome subcellular localization in at least one cell of at least one biological sample of said subject, or in any fraction of said cell, wherein at least one of said sample is obtained after the initiation of said treatment regimen;
(b) determining any one of:
(i) a disease relapse and/or loss of responsiveness, and/or drug-resistance, if at least one cell of said sample displays loss of proteasome nuclear localization, or maintained cytosolic localization; or
(ii) responsiveness or maintained responsiveness of said subject, if at least one cell of said sample displays maintained predominant proteasome nuclear localization; and
(c) ceasing a treatment regimen comprising at least one UPS-modulating agent of a subject displaying disease relapse and/or loss of responsiveness, or maintaining said treatment regimen, of a subject displaying responsiveness or maintained responsiveness.

62. The method according to claim 58, wherein said proliferative disorder is at least one hematological malignancy, and wherein said protein misfolding disorder is amyloidosis and any related conditions.

63. The method according to claim 58, wherein said hematological malignancy is MM, and wherein said method is for treating, inhibiting, reducing, eliminating, protecting or delaying the onset of MM, and/or any related conditions in a subject.

64. A method for treating, inhibiting, reducing, eliminating, protecting or delaying the onset of a cancer in a subject, by selectively modulating proteasome translocation to the cytosol of cancer cells of said subject, and/or for selective induction of apoptosis of cancer cells, and/or for predicting and assessing responsiveness of a subject suffering from a proliferative disorder to a selective modulator of proteasome translocation, the method comprising the step of:; said method further comprises the steps of:
(a) determining proteasome subcellular localization in at least one cell of at least one biological sample of said subject or in any fraction of said cell; and
(b) classifying said subject as a responder subject to said selective inhibitor of proteasome translocation, if proteasome subcellular localization is cytosolic or equally distributed in at least one cell of said at least one sample; and
(c) administering to a subject classified as a responder subject, a therapeutically effective amount of at least one selective inhibitor of proteasome translocation, or any composition comprising said selective inhibitor;
optionally, the method further comprising the step of:
(d) determining proteasome subcellular localization in at least one cell of a sample of a subject classified in step (b) as a responsive subject, and confirming responsiveness of said subject if proteasome subcellular localization is predominantly nuclear in at least one cell after contacting with said selective inhibitor of proteasome translocation.

65. A screening method for identifying at least one selective modulator of proteasome translocation, the method comprising the steps of:
(a) determining proteasome subcellular localization in at least one cell contacted with a candidate compound, optionally, under cellular stress conditions, or in any fraction of said cell;
(b) determining the subcellular localization of at least one control protein, in at least one cell contacted with said candidate compound, optionally, under cellular stress conditions, or in any fraction of said cell, wherein said at least one control protein is at least one exported control protein and/or at least one imported control protein; and
(c) determining that said candidate compound is:
(i) a selective inhibitor of proteasome translocation, if proteasome subcellular localization of (a) is predominantly nuclear and the subcellular localization of said at least one exported control protein of (b) is predominantly cytosolic or equally distributed in said at least one cell contacted with said candidate compound; or
(ii) a selective enhancer of proteasome translocation, if proteasome subcellular localization of (a) is predominantly cytosolic and the subcellular localization of said at least one imported control protein of (b) is predominantly nuclear in said at least one cell contacted with said candidate compound; optionally, wherein the import or export of said at least one control protein is mediated by at least one nucleocytoplasmic transport component.


SEQUENCE LISTING (8 sequences)

SEQ ID NO: 1 - protein, 11 aa, Artificial Sequence
  leucine-rich NES consensus sequence
  MISC_FEATURE (1)..(1): Xaa is any one of Leu, Ile, Val, Phe or Met
  MISC_FEATURE (2)..(4): Xaa is any amino acid and residue 4 may be absent
  MISC_FEATURE (5)..(5): Xaa is any one of Leu, Ile, Val, Phe or Met
  MISC_FEATURE (6)..(8): Xaa is any amino acid and residue 8 may be absent
  MISC_FEATURE (9)..(9): Xaa is any one of Leu, Ile, Val, Phe or Met
  MISC_FEATURE (10)..(10): Xaa is any amino acid
  MISC_FEATURE (11)..(11): Xaa is any one of Leu, Ile, Val, Phe or Met
XXXXXXXXXXX

SEQ ID NO: 2 - protein, 9 aa, Artificial Sequence
  NES sequence
LPPLERLTL

SEQ ID NO: 3 - DNA, 70 bp, Artificial Sequence
  NES encoding sequence derived from p62
gcccgtcggccgccgccaactacctgctctgtaccaactgccggaaagtgctgcggaaggataaaagaat

SEQ ID NO: 4 - protein, 18 aa, Artificial Sequence
  NES sequence derived from p62
ATQSLAEQMRKIALESEG

SEQ ID NO: 5 - protein, 7 aa, Artificial Sequence
  Classical NLS, (monopartite)
PKKKRKV

SEQ ID NO: 6 - protein, 16 aa, Artificial Sequence
  Classical NLS, (bipartite)
  MISC_FEATURE (3)..(12): Xaa is any amino acid
KRXXXXXXXXXXKKKL

SEQ ID NO: 7 - protein, 12 aa, Artificial Sequence
  Non-classical NLS
PRVRYNPYTTRP

SEQ ID NO: 8 - DNA, 24 bp, Artificial Sequence
  NLS encoding sequence derived SV40
ccaccaaagaagaagagaaaggtt